SEQUENCE LISTING (616 sequences)

SEQ ID NO: 1 - DNA, 21 bp, Artificial sequence
  Synthetic polynucleotide
  misc_feature (1)..(1): V is a UNA monomer
  misc_feature (2)..(19): N is any nucleotide
  misc_feature (20)..(20): V is a UNA monomer
  misc_feature (21)..(21): N is any nucleotide
vnnnnnnnnnnnnnnnnnnvn

SEQ ID NO: 2 - DNA, 21 bp, Artificial sequence
  Synthetic polynucleotide
  misc_feature (1)..(1): N is any nucleotide
  misc_feature (2)..(2): V is a UNA monomer
  misc_feature (3)..(21): N is any nucleotide
nvnnnnnnnnnnnnnnnnnnn

SEQ ID NO: 3 - DNA, 19 bp, Artificial sequence
  Synthetic polynucleotide
cuccaggugucauccauca

SEQ ID NO: 4 - DNA, 19 bp, Artificial sequence
  Synthetic polynucleotide
cugagguccagcucuccuu

SEQ ID NO: 5 - DNA, 19 bp, Artificial sequence
  Synthetic polynucleotide
ccugcagagaccucaaaag

SEQ ID NO: 6 - DNA, 19 bp, Artificial sequence
  Synthetic polynucleotide
cccaccugaacguggucaa

SEQ ID NO: 7 - DNA, 19 bp, Artificial sequence
  Synthetic polynucleotide
cuggucaagaucugugacu

SEQ ID NO: 8 - DNA, 19 bp, Artificial sequence
  Synthetic polynucleotide
aucuucaacagccucuaca

SEQ ID NO: 9 - DNA, 19 bp, Artificial sequence
  Synthetic polynucleotide
aguucuacaaugccaucaa

SEQ ID NO: 10 - DNA, 19 bp, Artificial sequence
  Synthetic polynucleotide
ccgacgagaucuaugagau

SEQ ID NO: 11 - DNA, 19 bp, Artificial sequence
  Synthetic polynucleotide
gguggcaccccuuacccag

SEQ ID NO: 12 - DNA, 19 bp, Artificial sequence
  Synthetic polynucleotide
caucuuugugccagauccc

SEQ ID NO: 13 - DNA, 19 bp, Artificial sequence
  Synthetic polynucleotide
cugcaaaaccaccauuggg

SEQ ID NO: 14 - DNA, 19 bp, Artificial sequence
  Synthetic polynucleotide
caugaguacaucuacgugg

SEQ ID NO: 15 - DNA, 19 bp, Artificial sequence
  Synthetic polynucleotide
gaguacaucuacguggacc

SEQ ID NO: 16 - DNA, 19 bp, Artificial sequence
  Synthetic polynucleotide
ccuggcucgagacaucaug

SEQ ID NO: 17 - DNA, 19 bp, Artificial sequence
  Synthetic polynucleotide
ucaacagccucuacaccac

SEQ ID NO: 18 - DNA, 19 bp, Artificial sequence
  Synthetic polynucleotide
gugggcggcugguggagcc

SEQ ID NO: 19 - DNA, 19 bp, Artificial sequence
  Synthetic polynucleotide
gcugucguggccggggcau

SEQ ID NO: 20 - DNA, 19 bp, Artificial sequence
  Synthetic polynucleotide
gagguggugagcacacugc

SEQ ID NO: 21 - DNA, 19 bp, Artificial sequence
  Synthetic polynucleotide
ggugaucucagccauccug

SEQ ID NO: 22 - DNA, 19 bp, Artificial sequence
  Synthetic polynucleotide
auugagucugugagcucug

SEQ ID NO: 23 - DNA, 19 bp, Artificial sequence
  Synthetic polynucleotide
gggggccugcaccaaagga

SEQ ID NO: 24 - DNA, 19 bp, Artificial sequence
  Synthetic polynucleotide
cugguggacuaccugcacc

SEQ ID NO: 25 - DNA, 19 bp, Artificial sequence
  Synthetic polynucleotide
gcuacauggacaugagcaa

SEQ ID NO: 26 - DNA, 19 bp, Artificial sequence
  Synthetic polynucleotide
caugcuggacaugaaagga

SEQ ID NO: 27 - DNA, 19 bp, Artificial sequence
  Synthetic polynucleotide
uggcccccuacgauaacua

SEQ ID NO: 28 - DNA, 19 bp, Artificial sequence
  Synthetic polynucleotide
cugccccugagaggaccua

SEQ ID NO: 29 - DNA, 19 bp, Artificial sequence
  Synthetic polynucleotide
ucgugggcuucagcuacca

SEQ ID NO: 30 - DNA, 19 bp, Artificial sequence
  Synthetic polynucleotide
ccuccaagaacugcgucca

SEQ ID NO: 31 - DNA, 19 bp, Artificial sequence
  Synthetic polynucleotide
gccaggaacgugcucaucu

SEQ ID NO: 32 - DNA, 19 bp, Artificial sequence
  Synthetic polynucleotide
cucaaacuacaucuccaaa

SEQ ID NO: 33 - DNA, 19 bp, Artificial sequence
  Synthetic polynucleotide
cuacucugggagaucuuca

SEQ ID NO: 34 - DNA, 19 bp, Artificial sequence
  Synthetic polynucleotide
gcauggcccagccugccca

SEQ ID NO: 35 - DNA, 19 bp, Artificial sequence
  Synthetic polynucleotide
ugggaagaaaaguuugaga

SEQ ID NO: 36 - DNA, 19 bp, Artificial sequence
  Synthetic polynucleotide
ucucccagcuggugcugcu

SEQ ID NO: 37 - DNA, 19 bp, Artificial sequence
  Synthetic polynucleotide
gcgaaggcuacaaaaagaa

SEQ ID NO: 38 - DNA, 19 bp, Artificial sequence
  Synthetic polynucleotide
guuucugaggagugaccac

SEQ ID NO: 39 - DNA, 19 bp, Artificial sequence
  Synthetic polynucleotide
cuccgauccccccuggaca

SEQ ID NO: 40 - DNA, 19 bp, Artificial sequence
  Synthetic polynucleotide
agcccaaugagagugacaa

SEQ ID NO: 41 - DNA, 19 bp, Artificial sequence
  Synthetic polynucleotide
uaccugaccccaaacccga

SEQ ID NO: 42 - DNA, 19 bp, Artificial sequence
  Synthetic polynucleotide
acccugaaugaagucaaca

SEQ ID NO: 43 - DNA, 19 bp, Artificial sequence
  Synthetic polynucleotide
gcccccuggagccccaaga

SEQ ID NO: 44 - DNA, 19 bp, Artificial sequence
  Synthetic polynucleotide
cagaggacagcuuccugua

SEQ ID NO: 45 - DNA, 19 bp, Artificial sequence
  Synthetic polynucleotide
cuuaggaggcaagaaaacu

SEQ ID NO: 46 - DNA, 19 bp, Artificial sequence
  Synthetic polynucleotide
gacucugaaccaggguucc

SEQ ID NO: 47 - DNA, 19 bp, Artificial sequence
  Synthetic polynucleotide
ccaagcuggucuggggcca

SEQ ID NO: 48 - DNA, 19 bp, Artificial sequence
  Synthetic polynucleotide
ugcacuggaccugcuauga

SEQ ID NO: 49 - DNA, 19 bp, Artificial sequence
  Synthetic polynucleotide
ccccaaggacacagaaaga

SEQ ID NO: 50 - DNA, 19 bp, Artificial sequence
  Synthetic polynucleotide
ucaccuagguuuacaaaua

SEQ ID NO: 51 - DNA, 19 bp, Artificial sequence
  Synthetic polynucleotide
uauaugcuguuaaguuuuu

SEQ ID NO: 52 - DNA, 19 bp, Artificial sequence
  Synthetic polynucleotide
gaaagauuuuaauauuaaa

SEQ ID NO: 53 - DNA, 19 bp, Artificial sequence
  Synthetic polynucleotide
ugauggaugacaccuggag

SEQ ID NO: 54 - DNA, 19 bp, Artificial sequence
  Synthetic polynucleotide
aaggagagcuggaccucag

SEQ ID NO: 55 - DNA, 19 bp, Artificial sequence
  Synthetic polynucleotide
cuuuugaggucucugcagg

SEQ ID NO: 56 - DNA, 19 bp, Artificial sequence
  Synthetic polynucleotide
uugaccacguucagguggg

SEQ ID NO: 57 - DNA, 19 bp, Artificial sequence
  Synthetic polynucleotide
uugaccacguucagguggg

SEQ ID NO: 58 - DNA, 19 bp, Artificial sequence
  Synthetic polynucleotide
uguagaggcuguugaagau

SEQ ID NO: 59 - DNA, 19 bp, Artificial sequence
  Synthetic polynucleotide
uugauggcauuguagaacu

SEQ ID NO: 60 - DNA, 19 bp, Artificial sequence
  Synthetic polynucleotide
aucucauagaucucgucgg

SEQ ID NO: 61 - DNA, 19 bp, Artificial sequence
  Synthetic polynucleotide
cuggguaaggggugccacc

SEQ ID NO: 62 - DNA, 19 bp, Artificial sequence
  Synthetic polynucleotide
gggaucuggcacaaagaug

SEQ ID NO: 63 - DNA, 19 bp, Artificial sequence
  Synthetic polynucleotide
cccaauggugguuuugcag

SEQ ID NO: 64 - DNA, 19 bp, Artificial sequence
  Synthetic polynucleotide
ccacguagauguacucaug

SEQ ID NO: 65 - DNA, 19 bp, Artificial sequence
  Synthetic polynucleotide
gguccacguagauguacuc

SEQ ID NO: 66 - DNA, 19 bp, Artificial sequence
  Synthetic polynucleotide
caugaugucucgagccagg

SEQ ID NO: 67 - DNA, 19 bp, Artificial sequence
  Synthetic polynucleotide
gugguguagaggcuguuga

SEQ ID NO: 68 - DNA, 19 bp, Artificial sequence
  Synthetic polynucleotide
ggcuccaccagccgcccac

SEQ ID NO: 69 - DNA, 19 bp, Artificial sequence
  Synthetic polynucleotide
augccccggccacgacagc

SEQ ID NO: 70 - DNA, 19 bp, Artificial sequence
  Synthetic polynucleotide
gcagugugcucaccaccuc

SEQ ID NO: 71 - DNA, 19 bp, Artificial sequence
  Synthetic polynucleotide
caggauggcugagaucacc

SEQ ID NO: 72 - DNA, 19 bp, Artificial sequence
  Synthetic polynucleotide
cagagcucacagacucaau

SEQ ID NO: 73 - DNA, 19 bp, Artificial sequence
  Synthetic polynucleotide
uccuuuggugcaggccccc

SEQ ID NO: 74 - DNA, 19 bp, Artificial sequence
  Synthetic polynucleotide
ggugcagguaguccaccag

SEQ ID NO: 75 - DNA, 19 bp, Artificial sequence
  Synthetic polynucleotide
uugcucauguccauguagc

SEQ ID NO: 76 - DNA, 19 bp, Artificial sequence
  Synthetic polynucleotide
uccuuucauguccagcaug

SEQ ID NO: 77 - DNA, 19 bp, Artificial sequence
  Synthetic polynucleotide
uaguuaucguagggggcca

SEQ ID NO: 78 - DNA, 19 bp, Artificial sequence
  Synthetic polynucleotide
uagguccucucaggggcag

SEQ ID NO: 79 - DNA, 19 bp, Artificial sequence
  Synthetic polynucleotide
ugguagcugaagcccacga

SEQ ID NO: 80 - DNA, 19 bp, Artificial sequence
  Synthetic polynucleotide
uggacgcaguucuuggagg

SEQ ID NO: 81 - DNA, 19 bp, Artificial sequence
  Synthetic polynucleotide
agaugagcacguuccuggc

SEQ ID NO: 82 - DNA, 19 bp, Artificial sequence
  Synthetic polynucleotide
uuuggagauguaguuugag

SEQ ID NO: 83 - DNA, 19 bp, Artificial sequence
  Synthetic polynucleotide
ugaagaucucccagaguag

SEQ ID NO: 84 - DNA, 19 bp, Artificial sequence
  Synthetic polynucleotide
ugggcaggcugggccaugc

SEQ ID NO: 85 - DNA, 19 bp, Artificial sequence
  Synthetic polynucleotide
ucucaaacuuuucuuccca

SEQ ID NO: 86 - DNA, 19 bp, Artificial sequence
  Synthetic polynucleotide
agcagcaccagcugggaga

SEQ ID NO: 87 - DNA, 19 bp, Artificial sequence
  Synthetic polynucleotide
uucuuuuuguagccuucgc

SEQ ID NO: 88 - DNA, 19 bp, Artificial sequence
  Synthetic polynucleotide
guggucacuccucagaaac

SEQ ID NO: 89 - DNA, 19 bp, Artificial sequence
  Synthetic polynucleotide
uguccaggggggaucggag

SEQ ID NO: 90 - DNA, 19 bp, Artificial sequence
  Synthetic polynucleotide
uugucacucucauugggcu

SEQ ID NO: 91 - DNA, 19 bp, Artificial sequence
  Synthetic polynucleotide
ucggguuuggggucaggua

SEQ ID NO: 92 - DNA, 19 bp, Artificial sequence
  Synthetic polynucleotide
uguugacuucauucagggu

SEQ ID NO: 93 - DNA, 19 bp, Artificial sequence
  Synthetic polynucleotide
ucuuggggcuccagggggc

SEQ ID NO: 94 - DNA, 19 bp, Artificial sequence
  Synthetic polynucleotide
uacaggaagcuguccucug

SEQ ID NO: 95 - DNA, 19 bp, Artificial sequence
  Synthetic polynucleotide
aguuuucuugccuccuaag

SEQ ID NO: 96 - DNA, 19 bp, Artificial sequence
  Synthetic polynucleotide
ggaacccugguucagaguc

SEQ ID NO: 97 - DNA, 19 bp, Artificial sequence
  Synthetic polynucleotide
uggccccagaccagcuugg

SEQ ID NO: 98 - DNA, 19 bp, Artificial sequence
  Synthetic polynucleotide
ucauagcagguccagugca

SEQ ID NO: 99 - DNA, 19 bp, Artificial sequence
  Synthetic polynucleotide
ucuuucuguguccuugggg

SEQ ID NO: 100 - DNA, 19 bp, Artificial sequence
  Synthetic polynucleotide
uauuuguaaaccuagguga

SEQ ID NO: 101 - DNA, 19 bp, Artificial sequence
  Synthetic polynucleotide
aaaaacuuaacagcauaua

SEQ ID NO: 102 - DNA, 19 bp, Artificial sequence
  Synthetic polynucleotide
uuuaauauuaaaaucuuuc

SEQ ID NO: 103 - DNA, 21 bp, Artificial sequence
  Synthetic polynucleotide
aucuucaacagccucuacaut

SEQ ID NO: 104 - DNA, 21 bp, Artificial sequence
  Synthetic polynucleotide
uguagaggcuguugaagauut

SEQ ID NO: 105 - DNA, 21 bp, Artificial sequence
  Synthetic polynucleotide
gagagcaucuucaacagccut

SEQ ID NO: 106 - DNA, 21 bp, Artificial sequence
  Synthetic polynucleotide
ggcuguugaagaugcucucut

SEQ ID NO: 107 - DNA, 21 bp, Artificial sequence
  Synthetic polynucleotide
gagcaucuucaacagccucut

SEQ ID NO: 108 - DNA, 21 bp, Artificial sequence
  Synthetic polynucleotide
gaggcuguugaagaugcucut

SEQ ID NO: 109 - DNA, 21 bp, Artificial sequence
  Synthetic polynucleotide
gcaucuucaacagccucuaut

SEQ ID NO: 110 - DNA, 21 bp, Artificial sequence
  Synthetic polynucleotide
uagaggcuguugaagaugcut

SEQ ID NO: 111 - DNA, 21 bp, Artificial sequence
  Synthetic polynucleotide
cuucaacagccucuacaccut

SEQ ID NO: 112 - DNA, 21 bp, Artificial sequence
  Synthetic polynucleotide
ucauagaucucgucggaggut

SEQ ID NO: 113 - DNA, 21 bp, Artificial sequence
  Synthetic polynucleotide
cuucaacagccucuacaccut

SEQ ID NO: 114 - DNA, 21 bp, Artificial sequence
  Synthetic polynucleotide
gugguguagaggcuguugaut

SEQ ID NO: 115 - DNA, 21 bp, Artificial sequence
  Synthetic polynucleotide
ccgacgagaucuaugagauut

SEQ ID NO: 116 - DNA, 21 bp, Artificial sequence
  Synthetic polynucleotide
aucucauagaucucgucggut

SEQ ID NO: 117 - DNA, 21 bp, Artificial sequence
  Synthetic polynucleotide
ugccuccgacgagaucuauut

SEQ ID NO: 118 - DNA, 21 bp, Artificial sequence
  Synthetic polynucleotide
auagaucucgucggaggcaut

SEQ ID NO: 119 - DNA, 21 bp, Artificial sequence
  Synthetic polynucleotide
gccuccgacgagaucuaugut

SEQ ID NO: 120 - DNA, 21 bp, Artificial sequence
  Synthetic polynucleotide
cauagaucucgucggaggcut

SEQ ID NO: 121 - DNA, 21 bp, Artificial sequence
  Synthetic polynucleotide
ccuccgacgagaucuaugaut

SEQ ID NO: 122 - DNA, 21 bp, Artificial sequence
  Synthetic polynucleotide
ucauagaucucgucggaggut

SEQ ID NO: 123 - DNA, 21 bp, Artificial sequence
  Synthetic polynucleotide
cuccgacgagaucuaugagut

SEQ ID NO: 124 - DNA, 21 bp, Artificial sequence
  Synthetic polynucleotide
cucauagaucucgucggagut

SEQ ID NO: 125 - DNA, 21 bp, Artificial sequence
  Synthetic polynucleotide
uccgacgagaucuaugagaut

SEQ ID NO: 126 - DNA, 21 bp, Artificial sequence
  Synthetic polynucleotide
ucucauagaucucgucggaut

SEQ ID NO: 127 - DNA, 21 bp, Artificial sequence
  Synthetic polynucleotide
cgacgagaucuaugagaucut

SEQ ID NO: 128 - DNA, 21 bp, Artificial sequence
  Synthetic polynucleotide
gaucucauagaucucgucgut

SEQ ID NO: 129 - DNA, 21 bp, Artificial sequence
  Synthetic polynucleotide
gacgagaucuaugagaucaut

SEQ ID NO: 130 - DNA, 21 bp, Artificial sequence
  Synthetic polynucleotide
ugaucucauagaucucgucut

SEQ ID NO: 131 - DNA, 21 bp, Artificial sequence
  Synthetic polynucleotide
acgagaucuaugagaucauut

SEQ ID NO: 132 - DNA, 21 bp, Artificial sequence
  Synthetic polynucleotide
augaucucauagaucucguut

SEQ ID NO: 133 - DNA, 21 bp, Artificial sequence
  Synthetic polynucleotide
cgagaucuaugagaucaugut

SEQ ID NO: 134 - DNA, 21 bp, Artificial sequence
  Synthetic polynucleotide
caugaucucauagaucucgut

SEQ ID NO: 135 - DNA, 21 bp, Artificial sequence
  Synthetic polynucleotide
gagaucuaugagaucaugcut

SEQ ID NO: 136 - DNA, 21 bp, Artificial sequence
  Synthetic polynucleotide
gcaugaucucauagaucucut

SEQ ID NO: 137 - DNA, 21 bp, Artificial sequence
  Synthetic polynucleotide
agaucuaugagaucaugcaut

SEQ ID NO: 138 - DNA, 21 bp, Artificial sequence
  Synthetic polynucleotide
ugcaugaucucauagaucuut

SEQ ID NO: 139 - DNA, 21 bp, Artificial sequence
  Synthetic polynucleotide
gaucuaugagaucaugcagut

SEQ ID NO: 140 - DNA, 21 bp, Artificial sequence
  Synthetic polynucleotide
cugcaugaucucauagaucut

SEQ ID NO: 141 - DNA, 21 bp, Artificial sequence
  Synthetic polynucleotide
aucuaugagaucaugcagaut

SEQ ID NO: 142 - DNA, 21 bp, Artificial sequence
  Synthetic polynucleotide
ucugcaugaucucauagauut

SEQ ID NO: 143 - DNA, 21 bp, Artificial sequence
  Synthetic polynucleotide
ucuaugagaucaugcagaaut

SEQ ID NO: 144 - DNA, 21 bp, Artificial sequence
  Synthetic polynucleotide
uucugcaugaucucauagaut

SEQ ID NO: 145 - DNA, 21 bp, Artificial sequence
  Synthetic polynucleotide
cuaugagaucaugcagaagut

SEQ ID NO: 146 - DNA, 21 bp, Artificial sequence
  Synthetic polynucleotide
cuucugcaugaucucauagut

SEQ ID NO: 147 - DNA, 21 bp, Artificial sequence
  Synthetic polynucleotide
gcuacauggacaugagcaaut

SEQ ID NO: 148 - DNA, 21 bp, Artificial sequence
  Synthetic polynucleotide
ccugaccggggagagcgacut

SEQ ID NO: 149 - DNA, 21 bp, Artificial sequence
  Synthetic polynucleotide
cugaccggggagagcgacgut

SEQ ID NO: 150 - DNA, 21 bp, Artificial sequence
  Synthetic polynucleotide
ugaccggggagagcgacggut

SEQ ID NO: 151 - DNA, 21 bp, Artificial sequence
  Synthetic polynucleotide
gaccggggagagcgacgguut

SEQ ID NO: 152 - DNA, 21 bp, Artificial sequence
  Synthetic polynucleotide
accggggagagcgacggugut

SEQ ID NO: 153 - DNA, 21 bp, Artificial sequence
  Synthetic polynucleotide
ccggggagagcgacgguggut

SEQ ID NO: 154 - DNA, 21 bp, Artificial sequence
  Synthetic polynucleotide
cggggagagcgacgguggcut

SEQ ID NO: 155 - DNA, 21 bp, Artificial sequence
  Synthetic polynucleotide
ggggagagcgacgguggcuut

SEQ ID NO: 156 - DNA, 21 bp, Artificial sequence
  Synthetic polynucleotide
gggagagcgacgguggcuaut

SEQ ID NO: 157 - DNA, 21 bp, Artificial sequence
  Synthetic polynucleotide
ggagagcgacgguggcuacut

SEQ ID NO: 158 - DNA, 21 bp, Artificial sequence
  Synthetic polynucleotide
gagagcgacgguggcuacaut

SEQ ID NO: 159 - DNA, 21 bp, Artificial sequence
  Synthetic polynucleotide
agagcgacgguggcuacauut

SEQ ID NO: 160 - DNA, 21 bp, Artificial sequence
  Synthetic polynucleotide
gagcgacgguggcuacaugut

SEQ ID NO: 161 - DNA, 21 bp, Artificial sequence
  Synthetic polynucleotide
agcgacgguggcuacauggut

SEQ ID NO: 162 - DNA, 21 bp, Artificial sequence
  Synthetic polynucleotide
gcgacgguggcuacauggaut

SEQ ID NO: 163 - DNA, 21 bp, Artificial sequence
  Synthetic polynucleotide
cgacgguggcuacauggacut

SEQ ID NO: 164 - DNA, 21 bp, Artificial sequence
  Synthetic polynucleotide
gacgguggcuacauggacaut

SEQ ID NO: 165 - DNA, 21 bp, Artificial sequence
  Synthetic polynucleotide
acgguggcuacauggacauut

SEQ ID NO: 166 - DNA, 21 bp, Artificial sequence
  Synthetic polynucleotide
cgguggcuacauggacaugut

SEQ ID NO: 167 - DNA, 21 bp, Artificial sequence
  Synthetic polynucleotide
gguggcuacauggacaugaut

SEQ ID NO: 168 - DNA, 21 bp, Artificial sequence
  Synthetic polynucleotide
guggcuacauggacaugagut

SEQ ID NO: 169 - DNA, 21 bp, Artificial sequence
  Synthetic polynucleotide
uggcuacauggacaugagcut

SEQ ID NO: 170 - DNA, 21 bp, Artificial sequence
  Synthetic polynucleotide
ggcuacauggacaugagcaut

SEQ ID NO: 171 - DNA, 21 bp, Artificial sequence
  Synthetic polynucleotide
cuacauggacaugagcaagut

SEQ ID NO: 172 - DNA, 21 bp, Artificial sequence
  Synthetic polynucleotide
uacauggacaugagcaaggut

SEQ ID NO: 173 - DNA, 21 bp, Artificial sequence
  Synthetic polynucleotide
acauggacaugagcaaggaut

SEQ ID NO: 174 - DNA, 21 bp, Artificial sequence
  Synthetic polynucleotide
cauggacaugagcaaggacut

SEQ ID NO: 175 - DNA, 21 bp, Artificial sequence
  Synthetic polynucleotide
auggacaugagcaaggacgut

SEQ ID NO: 176 - DNA, 21 bp, Artificial sequence
  Synthetic polynucleotide
uggacaugagcaaggacgaut

SEQ ID NO: 177 - DNA, 21 bp, Artificial sequence
  Synthetic polynucleotide
ggacaugagcaaggacgaaut

SEQ ID NO: 178 - DNA, 21 bp, Artificial sequence
  Synthetic polynucleotide
gacaugagcaaggacgaauut

SEQ ID NO: 179 - DNA, 21 bp, Artificial sequence
  Synthetic polynucleotide
acaugagcaaggacgaaucut

SEQ ID NO: 180 - DNA, 21 bp, Artificial sequence
  Synthetic polynucleotide
uugcucauguccauguagcut

SEQ ID NO: 181 - DNA, 21 bp, Artificial sequence
  Synthetic polynucleotide
gucgcucuccccggucaggut

SEQ ID NO: 182 - DNA, 21 bp, Artificial sequence
  Synthetic polynucleotide
cgucgcucuccccggucagut

SEQ ID NO: 183 - DNA, 21 bp, Artificial sequence
  Synthetic polynucleotide
ccgucgcucuccccggucaut

SEQ ID NO: 184 - DNA, 21 bp, Artificial sequence
  Synthetic polynucleotide
accgucgcucuccccggucut

SEQ ID NO: 185 - DNA, 21 bp, Artificial sequence
  Synthetic polynucleotide
caccgucgcucuccccgguut

SEQ ID NO: 186 - DNA, 21 bp, Artificial sequence
  Synthetic polynucleotide
ccaccgucgcucuccccggut

SEQ ID NO: 187 - DNA, 21 bp, Artificial sequence
  Synthetic polynucleotide
gccaccgucgcucuccccgut

SEQ ID NO: 188 - DNA, 21 bp, Artificial sequence
  Synthetic polynucleotide
agccaccgucgcucuccccut

SEQ ID NO: 189 - DNA, 21 bp, Artificial sequence
  Synthetic polynucleotide
uagccaccgucgcucucccut

SEQ ID NO: 190 - DNA, 21 bp, Artificial sequence
  Synthetic polynucleotide
guagccaccgucgcucuccut

SEQ ID NO: 191 - DNA, 21 bp, Artificial sequence
  Synthetic polynucleotide
uguagccaccgucgcucucut

SEQ ID NO: 192 - DNA, 21 bp, Artificial sequence
  Synthetic polynucleotide
auguagccaccgucgcucuut

SEQ ID NO: 193 - DNA, 21 bp, Artificial sequence
  Synthetic polynucleotide
cauguagccaccgucgcucut

SEQ ID NO: 194 - DNA, 21 bp, Artificial sequence
  Synthetic polynucleotide
ccauguagccaccgucgcuut

SEQ ID NO: 195 - DNA, 21 bp, Artificial sequence
  Synthetic polynucleotide
uccauguagccaccgucgcut

SEQ ID NO: 196 - DNA, 21 bp, Artificial sequence
  Synthetic polynucleotide
guccauguagccaccgucgut

SEQ ID NO: 197 - DNA, 21 bp, Artificial sequence
  Synthetic polynucleotide
uguccauguagccaccgucut

SEQ ID NO: 198 - DNA, 21 bp, Artificial sequence
  Synthetic polynucleotide
auguccauguagccaccguut

SEQ ID NO: 199 - DNA, 21 bp, Artificial sequence
  Synthetic polynucleotide
cauguccauguagccaccgut

SEQ ID NO: 200 - DNA, 21 bp, Artificial sequence
  Synthetic polynucleotide
ucauguccauguagccaccut

SEQ ID NO: 201 - DNA, 21 bp, Artificial sequence
  Synthetic polynucleotide
cucauguccauguagccacut

SEQ ID NO: 202 - DNA, 21 bp, Artificial sequence
  Synthetic polynucleotide
gcucauguccauguagccaut

SEQ ID NO: 203 - DNA, 21 bp, Artificial sequence
  Synthetic polynucleotide
ugcucauguccauguagccut

SEQ ID NO: 204 - DNA, 21 bp, Artificial sequence
  Synthetic polynucleotide
cuugcucauguccauguagut

SEQ ID NO: 205 - DNA, 21 bp, Artificial sequence
  Synthetic polynucleotide
ccuugcucauguccauguaut

SEQ ID NO: 206 - DNA, 21 bp, Artificial sequence
  Synthetic polynucleotide
uccuugcucauguccauguut

SEQ ID NO: 207 - DNA, 21 bp, Artificial sequence
  Synthetic polynucleotide
guccuugcucauguccaugut

SEQ ID NO: 208 - DNA, 21 bp, Artificial sequence
  Synthetic polynucleotide
cguccuugcucauguccauut

SEQ ID NO: 209 - DNA, 21 bp, Artificial sequence
  Synthetic polynucleotide
ucguccuugcucauguccaut

SEQ ID NO: 210 - DNA, 21 bp, Artificial sequence
  Synthetic polynucleotide
uucguccuugcucauguccut

SEQ ID NO: 211 - DNA, 21 bp, Artificial sequence
  Synthetic polynucleotide
auucguccuugcucaugucut

SEQ ID NO: 212 - DNA, 21 bp, Artificial sequence
  Synthetic polynucleotide
gauucguccuugcucauguut

SEQ ID NO: 213 - DNA, 21 bp, Artificial sequence
  Synthetic polynucleotide
agcccaaugagagugacaaut

SEQ ID NO: 214 - DNA, 21 bp, Artificial sequence
  Synthetic polynucleotide
cagcuccguccucuacaccut

SEQ ID NO: 215 - DNA, 21 bp, Artificial sequence
  Synthetic polynucleotide
agcuccguccucuacaccgut

SEQ ID NO: 216 - DNA, 21 bp, Artificial sequence
  Synthetic polynucleotide
gcuccguccucuacaccgcut

SEQ ID NO: 217 - DNA, 21 bp, Artificial sequence
  Synthetic polynucleotide
cuccguccucuacaccgccut

SEQ ID NO: 218 - DNA, 21 bp, Artificial sequence
  Synthetic polynucleotide
uccguccucuacaccgccgut

SEQ ID NO: 219 - DNA, 21 bp, Artificial sequence
  Synthetic polynucleotide
ccguccucuacaccgccguut

SEQ ID NO: 220 - DNA, 21 bp, Artificial sequence
  Synthetic polynucleotide
cguccucuacaccgccgugut

SEQ ID NO: 221 - DNA, 21 bp, Artificial sequence
  Synthetic polynucleotide
guccucuacaccgccgugcut

SEQ ID NO: 222 - DNA, 21 bp, Artificial sequence
  Synthetic polynucleotide
uccucuacaccgccgugcaut

SEQ ID NO: 223 - DNA, 21 bp, Artificial sequence
  Synthetic polynucleotide
ccucuacaccgccgugcagut

SEQ ID NO: 224 - DNA, 21 bp, Artificial sequence
  Synthetic polynucleotide
cucuacaccgccgugcagcut

SEQ ID NO: 225 - DNA, 21 bp, Artificial sequence
  Synthetic polynucleotide
ucuacaccgccgugcagccut

SEQ ID NO: 226 - DNA, 21 bp, Artificial sequence
  Synthetic polynucleotide
cuacaccgccgugcagcccut

SEQ ID NO: 227 - DNA, 21 bp, Artificial sequence
  Synthetic polynucleotide
uacaccgccgugcagcccaut

SEQ ID NO: 228 - DNA, 21 bp, Artificial sequence
  Synthetic polynucleotide
acaccgccgugcagcccaaut

SEQ ID NO: 229 - DNA, 21 bp, Artificial sequence
  Synthetic polynucleotide
caccgccgugcagcccaauut

SEQ ID NO: 230 - DNA, 21 bp, Artificial sequence
  Synthetic polynucleotide
accgccgugcagcccaaugut

SEQ ID NO: 231 - DNA, 21 bp, Artificial sequence
  Synthetic polynucleotide
ccgccgugcagcccaaugaut

SEQ ID NO: 232 - DNA, 21 bp, Artificial sequence
  Synthetic polynucleotide
cgccgugcagcccaaugagut

SEQ ID NO: 233 - DNA, 21 bp, Artificial sequence
  Synthetic polynucleotide
gccgugcagcccaaugagaut

SEQ ID NO: 234 - DNA, 21 bp, Artificial sequence
  Synthetic polynucleotide
ccgugcagcccaaugagagut

SEQ ID NO: 235 - DNA, 21 bp, Artificial sequence
  Synthetic polynucleotide
cgugcagcccaaugagaguut

SEQ ID NO: 236 - DNA, 21 bp, Artificial sequence
  Synthetic polynucleotide
gugcagcccaaugagagugut

SEQ ID NO: 237 - DNA, 21 bp, Artificial sequence
  Synthetic polynucleotide
ugcagcccaaugagagugaut

SEQ ID NO: 238 - DNA, 21 bp, Artificial sequence
  Synthetic polynucleotide
gcagcccaaugagagugacut

SEQ ID NO: 239 - DNA, 21 bp, Artificial sequence
  Synthetic polynucleotide
cagcccaaugagagugacaut

SEQ ID NO: 240 - DNA, 21 bp, Artificial sequence
  Synthetic polynucleotide
gcccaaugagagugacaacut

SEQ ID NO: 241 - DNA, 21 bp, Artificial sequence
  Synthetic polynucleotide
cccaaugagagugacaacgut

SEQ ID NO: 242 - DNA, 21 bp, Artificial sequence
  Synthetic polynucleotide
ccaaugagagugacaacgaut

SEQ ID NO: 243 - DNA, 21 bp, Artificial sequence
  Synthetic polynucleotide
caaugagagugacaacgacut

SEQ ID NO: 244 - DNA, 21 bp, Artificial sequence
  Synthetic polynucleotide
aaugagagugacaacgacuut

SEQ ID NO: 245 - DNA, 21 bp, Artificial sequence
  Synthetic polynucleotide
augagagugacaacgacuaut

SEQ ID NO: 246 - DNA, 21 bp, Artificial sequence
  Synthetic polynucleotide
ugagagugacaacgacuacut

SEQ ID NO: 247 - DNA, 21 bp, Artificial sequence
  Synthetic polynucleotide
gagagugacaacgacuacaut

SEQ ID NO: 248 - DNA, 21 bp, Artificial sequence
  Synthetic polynucleotide
agagugacaacgacuacauut

SEQ ID NO: 249 - DNA, 21 bp, Artificial sequence
  Synthetic polynucleotide
gagugacaacgacuacaucut

SEQ ID NO: 250 - DNA, 21 bp, Artificial sequence
  Synthetic polynucleotide
agugacaacgacuacaucaut

SEQ ID NO: 251 - DNA, 21 bp, Artificial sequence
  Synthetic polynucleotide
gugacaacgacuacaucauut

SEQ ID NO: 252 - DNA, 21 bp, Artificial sequence
  Synthetic polynucleotide
ugacaacgacuacaucaucut

SEQ ID NO: 253 - DNA, 21 bp, Artificial sequence
  Synthetic polynucleotide
gacaacgacuacaucauccut

SEQ ID NO: 254 - DNA, 21 bp, Artificial sequence
  Synthetic polynucleotide
acaacgacuacaucaucccut

SEQ ID NO: 255 - DNA, 21 bp, Artificial sequence
  Synthetic polynucleotide
caacgacuacaucauccccut

SEQ ID NO: 256 - DNA, 21 bp, Artificial sequence
  Synthetic polynucleotide
uugucacucucauugggcuut

SEQ ID NO: 257 - DNA, 21 bp, Artificial sequence
  Synthetic polynucleotide
gguguagaggacggagcugut

SEQ ID NO: 258 - DNA, 21 bp, Artificial sequence
  Synthetic polynucleotide
cgguguagaggacggagcuut

SEQ ID NO: 259 - DNA, 21 bp, Artificial sequence
  Synthetic polynucleotide
gcgguguagaggacggagcut

SEQ ID NO: 260 - DNA, 21 bp, Artificial sequence
  Synthetic polynucleotide
ggcgguguagaggacggagut

SEQ ID NO: 261 - DNA, 21 bp, Artificial sequence
  Synthetic polynucleotide
cggcgguguagaggacggaut

SEQ ID NO: 262 - DNA, 21 bp, Artificial sequence
  Synthetic polynucleotide
acggcgguguagaggacggut

SEQ ID NO: 263 - DNA, 21 bp, Artificial sequence
  Synthetic polynucleotide
cacggcgguguagaggacgut

SEQ ID NO: 264 - DNA, 21 bp, Artificial sequence
  Synthetic polynucleotide
gcacggcgguguagaggacut

SEQ ID NO: 265 - DNA, 21 bp, Artificial sequence
  Synthetic polynucleotide
ugcacggcgguguagaggaut

SEQ ID NO: 266 - DNA, 21 bp, Artificial sequence
  Synthetic polynucleotide
cugcacggcgguguagaggut

SEQ ID NO: 267 - DNA, 21 bp, Artificial sequence
  Synthetic polynucleotide
gcugcacggcgguguagagut

SEQ ID NO: 268 - DNA, 21 bp, Artificial sequence
  Synthetic polynucleotide
ggcugcacggcgguguagaut

SEQ ID NO: 269 - DNA, 21 bp, Artificial sequence
  Synthetic polynucleotide
gggcugcacggcgguguagut

SEQ ID NO: 270 - DNA, 21 bp, Artificial sequence
  Synthetic polynucleotide
ugggcugcacggcgguguaut

SEQ ID NO: 271 - DNA, 21 bp, Artificial sequence
  Synthetic polynucleotide
uugggcugcacggcgguguut

SEQ ID NO: 272 - DNA, 21 bp, Artificial sequence
  Synthetic polynucleotide
auugggcugcacggcggugut

SEQ ID NO: 273 - DNA, 21 bp, Artificial sequence
  Synthetic polynucleotide
cauugggcugcacggcgguut

SEQ ID NO: 274 - DNA, 21 bp, Artificial sequence
  Synthetic polynucleotide
ucauugggcugcacggcggut

SEQ ID NO: 275 - DNA, 21 bp, Artificial sequence
  Synthetic polynucleotide
cucauugggcugcacggcgut

SEQ ID NO: 276 - DNA, 21 bp, Artificial sequence
  Synthetic polynucleotide
ucucauugggcugcacggcut

SEQ ID NO: 277 - DNA, 21 bp, Artificial sequence
  Synthetic polynucleotide
cucucauugggcugcacggut

SEQ ID NO: 278 - DNA, 21 bp, Artificial sequence
  Synthetic polynucleotide
acucucauugggcugcacgut

SEQ ID NO: 279 - DNA, 21 bp, Artificial sequence
  Synthetic polynucleotide
cacucucauugggcugcacut

SEQ ID NO: 280 - DNA, 21 bp, Artificial sequence
  Synthetic polynucleotide
ucacucucauugggcugcaut

SEQ ID NO: 281 - DNA, 21 bp, Artificial sequence
  Synthetic polynucleotide
gucacucucauugggcugcut

SEQ ID NO: 282 - DNA, 21 bp, Artificial sequence
  Synthetic polynucleotide
ugucacucucauugggcugut

SEQ ID NO: 283 - DNA, 21 bp, Artificial sequence
  Synthetic polynucleotide
guugucacucucauugggcut

SEQ ID NO: 284 - DNA, 21 bp, Artificial sequence
  Synthetic polynucleotide
cguugucacucucauugggut

SEQ ID NO: 285 - DNA, 21 bp, Artificial sequence
  Synthetic polynucleotide
ucguugucacucucauuggut

SEQ ID NO: 286 - DNA, 21 bp, Artificial sequence
  Synthetic polynucleotide
gucguugucacucucauugut

SEQ ID NO: 287 - DNA, 21 bp, Artificial sequence
  Synthetic polynucleotide
agucguugucacucucauuut

SEQ ID NO: 288 - DNA, 21 bp, Artificial sequence
  Synthetic polynucleotide
uagucguugucacucucauut

SEQ ID NO: 289 - DNA, 21 bp, Artificial sequence
  Synthetic polynucleotide
guagucguugucacucucaut

SEQ ID NO: 290 - DNA, 21 bp, Artificial sequence
  Synthetic polynucleotide
uguagucguugucacucucut

SEQ ID NO: 291 - DNA, 21 bp, Artificial sequence
  Synthetic polynucleotide
auguagucguugucacucuut

SEQ ID NO: 292 - DNA, 21 bp, Artificial sequence
  Synthetic polynucleotide
gauguagucguugucacucut

SEQ ID NO: 293 - DNA, 21 bp, Artificial sequence
  Synthetic polynucleotide
ugauguagucguugucacuut

SEQ ID NO: 294 - DNA, 21 bp, Artificial sequence
  Synthetic polynucleotide
augauguagucguugucacut

SEQ ID NO: 295 - DNA, 21 bp, Artificial sequence
  Synthetic polynucleotide
gaugauguagucguugucaut

SEQ ID NO: 296 - DNA, 21 bp, Artificial sequence
  Synthetic polynucleotide
ggaugauguagucguugucut

SEQ ID NO: 297 - DNA, 21 bp, Artificial sequence
  Synthetic polynucleotide
gggaugauguagucguuguut

SEQ ID NO: 298 - DNA, 21 bp, Artificial sequence
  Synthetic polynucleotide
ggggaugauguagucguugut

SEQ ID NO: 299 - DNA, 21 bp, Artificial sequence
  Synthetic polynucleotide
acccugaaugaagucaacaut

SEQ ID NO: 300 - DNA, 21 bp, Artificial sequence
  Synthetic polynucleotide
agccagcuccacccugaauut

SEQ ID NO: 301 - DNA, 21 bp, Artificial sequence
  Synthetic polynucleotide
gccagcuccacccugaaugut

SEQ ID NO: 302 - DNA, 21 bp, Artificial sequence
  Synthetic polynucleotide
ccagcuccacccugaaugaut

SEQ ID NO: 303 - DNA, 21 bp, Artificial sequence
  Synthetic polynucleotide
cagcuccacccugaaugaaut

SEQ ID NO: 304 - DNA, 21 bp, Artificial sequence
  Synthetic polynucleotide
agcuccacccugaaugaagut

SEQ ID NO: 305 - DNA, 21 bp, Artificial sequence
  Synthetic polynucleotide
gcuccacccugaaugaaguut

SEQ ID NO: 306 - DNA, 21 bp, Artificial sequence
  Synthetic polynucleotide
cuccacccugaaugaagucut

SEQ ID NO: 307 - DNA, 21 bp, Artificial sequence
  Synthetic polynucleotide
uccacccugaaugaagucaut

SEQ ID NO: 308 - DNA, 21 bp, Artificial sequence
  Synthetic polynucleotide
ccacccugaaugaagucaaut

SEQ ID NO: 309 - DNA, 21 bp, Artificial sequence
  Synthetic polynucleotide
cacccugaaugaagucaacut

SEQ ID NO: 310 - DNA, 21 bp, Artificial sequence
  Synthetic polynucleotide
cccugaaugaagucaacacut

SEQ ID NO: 311 - DNA, 21 bp, Artificial sequence
  Synthetic polynucleotide
ccugaaugaagucaacaccut

SEQ ID NO: 312 - DNA, 21 bp, Artificial sequence
  Synthetic polynucleotide
cugaaugaagucaacaccuut

SEQ ID NO: 313 - DNA, 21 bp, Artificial sequence
  Synthetic polynucleotide
ugaaugaagucaacaccucut

SEQ ID NO: 314 - DNA, 21 bp, Artificial sequence
  Synthetic polynucleotide
gaaugaagucaacaccuccut

SEQ ID NO: 315 - DNA, 21 bp, Artificial sequence
  Synthetic polynucleotide
aaugaagucaacaccuccuut

SEQ ID NO: 316 - DNA, 21 bp, Artificial sequence
  Synthetic polynucleotide
augaagucaacaccuccucut

SEQ ID NO: 317 - DNA, 21 bp, Artificial sequence
  Synthetic polynucleotide
uguugacuucauucaggguut

SEQ ID NO: 318 - DNA, 21 bp, Artificial sequence
  Synthetic polynucleotide
auucaggguggagcuggcuut

SEQ ID NO: 319 - DNA, 21 bp, Artificial sequence
  Synthetic polynucleotide
cauucaggguggagcuggcut

SEQ ID NO: 320 - DNA, 21 bp, Artificial sequence
  Synthetic polynucleotide
ucauucaggguggagcuggut

SEQ ID NO: 321 - DNA, 21 bp, Artificial sequence
  Synthetic polynucleotide
uucauucaggguggagcugut

SEQ ID NO: 322 - DNA, 21 bp, Artificial sequence
  Synthetic polynucleotide
cuucauucaggguggagcuut

SEQ ID NO: 323 - DNA, 21 bp, Artificial sequence
  Synthetic polynucleotide
acuucauucaggguggagcut

SEQ ID NO: 324 - DNA, 21 bp, Artificial sequence
  Synthetic polynucleotide
gacuucauucaggguggagut

SEQ ID NO: 325 - DNA, 21 bp, Artificial sequence
  Synthetic polynucleotide
ugacuucauucaggguggaut

SEQ ID NO: 326 - DNA, 21 bp, Artificial sequence
  Synthetic polynucleotide
uugacuucauucaggguggut

SEQ ID NO: 327 - DNA, 21 bp, Artificial sequence
  Synthetic polynucleotide
guugacuucauucagggugut

SEQ ID NO: 328 - DNA, 21 bp, Artificial sequence
  Synthetic polynucleotide
guguugacuucauucagggut

SEQ ID NO: 329 - DNA, 21 bp, Artificial sequence
  Synthetic polynucleotide
gguguugacuucauucaggut

SEQ ID NO: 330 - DNA, 21 bp, Artificial sequence
  Synthetic polynucleotide
agguguugacuucauucagut

SEQ ID NO: 331 - DNA, 21 bp, Artificial sequence
  Synthetic polynucleotide
gagguguugacuucauucaut

SEQ ID NO: 332 - DNA, 21 bp, Artificial sequence
  Synthetic polynucleotide
ggagguguugacuucauucut

SEQ ID NO: 333 - DNA, 21 bp, Artificial sequence
  Synthetic polynucleotide
aggagguguugacuucauuut

SEQ ID NO: 334 - DNA, 21 bp, Artificial sequence
  Synthetic polynucleotide
gaggagguguugacuucauut

SEQ ID NO: 335 - DNA, 21 bp, Artificial sequence
  Synthetic polynucleotide
ucaccuagguuuacaaauaut

SEQ ID NO: 336 - DNA, 21 bp, Artificial sequence
  Synthetic polynucleotide
aaucaccuagguuuacaaaut

SEQ ID NO: 337 - DNA, 21 bp, Artificial sequence
  Synthetic polynucleotide
aucaccuagguuuacaaauut

SEQ ID NO: 338 - DNA, 21 bp, Artificial sequence
  Synthetic polynucleotide
caccuagguuuacaaauacut

SEQ ID NO: 339 - DNA, 21 bp, Artificial sequence
  Synthetic polynucleotide
accuagguuuacaaauacuut

SEQ ID NO: 340 - DNA, 21 bp, Artificial sequence
  Synthetic polynucleotide
ccuagguuuacaaauacuuut

SEQ ID NO: 341 - DNA, 21 bp, Artificial sequence
  Synthetic polynucleotide
uauuuguaaaccuaggugaut

SEQ ID NO: 342 - DNA, 21 bp, Artificial sequence
  Synthetic polynucleotide
uuuguaaaccuaggugauuut

SEQ ID NO: 343 - DNA, 21 bp, Artificial sequence
  Synthetic polynucleotide
auuuguaaaccuaggugauut

SEQ ID NO: 344 - DNA, 21 bp, Artificial sequence
  Synthetic polynucleotide
guauuuguaaaccuaggugut

SEQ ID NO: 345 - DNA, 21 bp, Artificial sequence
  Synthetic polynucleotide
aguauuuguaaaccuagguut

SEQ ID NO: 346 - DNA, 21 bp, Artificial sequence
  Synthetic polynucleotide
aaguauuuguaaaccuaggut

SEQ ID NO: 347 - DNA, 21 bp, Artificial sequence
  Synthetic polynucleotide
gcuacauggacaugagcaaut

SEQ ID NO: 348 - DNA, 21 bp, Artificial sequence
  Synthetic polynucleotide
ccugaccggggagagcgacut

SEQ ID NO: 349 - DNA, 21 bp, Artificial sequence
  Synthetic polynucleotide
cugaccggggagagcgacgut

SEQ ID NO: 350 - DNA, 21 bp, Artificial sequence
  Synthetic polynucleotide
ugaccggggagagcgacggut

SEQ ID NO: 351 - DNA, 21 bp, Artificial sequence
  Synthetic polynucleotide
gaccggggagagcgacgguut

SEQ ID NO: 352 - DNA, 21 bp, Artificial sequence
  Synthetic polynucleotide
accggggagagcgacggugut

SEQ ID NO: 353 - DNA, 21 bp, Artificial sequence
  Synthetic polynucleotide
ccggggagagcgacgguggut

SEQ ID NO: 354 - DNA, 21 bp, Artificial sequence
  Synthetic polynucleotide
cggggagagcgacgguggcut

SEQ ID NO: 355 - DNA, 21 bp, Artificial sequence
  Synthetic polynucleotide
ggggagagcgacgguggcuut

SEQ ID NO: 356 - DNA, 21 bp, Artificial sequence
  Synthetic polynucleotide
gggagagcgacgguggcuaut

SEQ ID NO: 357 - DNA, 21 bp, Artificial sequence
  Synthetic polynucleotide
ggagagcgacgguggcuacut

SEQ ID NO: 358 - DNA, 21 bp, Artificial sequence
  Synthetic polynucleotide
gagagcgacgguggcuacaut

SEQ ID NO: 359 - DNA, 21 bp, Artificial sequence
  Synthetic polynucleotide
agagcgacgguggcuacauut

SEQ ID NO: 360 - DNA, 21 bp, Artificial sequence
  Synthetic polynucleotide
gagcgacgguggcuacaugut

SEQ ID NO: 361 - DNA, 21 bp, Artificial sequence
  Synthetic polynucleotide
agcgacgguggcuacauggut

SEQ ID NO: 362 - DNA, 21 bp, Artificial sequence
  Synthetic polynucleotide
gcgacgguggcuacauggaut

SEQ ID NO: 363 - DNA, 21 bp, Artificial sequence
  Synthetic polynucleotide
cgacgguggcuacauggacut

SEQ ID NO: 364 - DNA, 21 bp, Artificial sequence
  Synthetic polynucleotide
gacgguggcuacauggacaut

SEQ ID NO: 365 - DNA, 21 bp, Artificial sequence
  Synthetic polynucleotide
acgguggcuacauggacauut

SEQ ID NO: 366 - DNA, 21 bp, Artificial sequence
  Synthetic polynucleotide
cgguggcuacauggacaugut

SEQ ID NO: 367 - DNA, 21 bp, Artificial sequence
  Synthetic polynucleotide
gguggcuacauggacaugaut

SEQ ID NO: 368 - DNA, 21 bp, Artificial sequence
  Synthetic polynucleotide
guggcuacauggacaugagut

SEQ ID NO: 369 - DNA, 21 bp, Artificial sequence
  Synthetic polynucleotide
uggcuacauggacaugagcut

SEQ ID NO: 370 - DNA, 21 bp, Artificial sequence
  Synthetic polynucleotide
ggcuacauggacaugagcaut

SEQ ID NO: 371 - DNA, 21 bp, Artificial sequence
  Synthetic polynucleotide
cuacauggacaugagcaagut

SEQ ID NO: 372 - DNA, 21 bp, Artificial sequence
  Synthetic polynucleotide
uacauggacaugagcaaggut

SEQ ID NO: 373 - DNA, 21 bp, Artificial sequence
  Synthetic polynucleotide
acauggacaugagcaaggaut

SEQ ID NO: 374 - DNA, 21 bp, Artificial sequence
  Synthetic polynucleotide
cauggacaugagcaaggacut

SEQ ID NO: 375 - DNA, 21 bp, Artificial sequence
  Synthetic polynucleotide
auggacaugagcaaggacgut

SEQ ID NO: 376 - DNA, 21 bp, Artificial sequence
  Synthetic polynucleotide
uggacaugagcaaggacgaut

SEQ ID NO: 377 - DNA, 21 bp, Artificial sequence
  Synthetic polynucleotide
ggacaugagcaaggacgaaut

SEQ ID NO: 378 - DNA, 21 bp, Artificial sequence
  Synthetic polynucleotide
gacaugagcaaggacgaauut

SEQ ID NO: 379 - DNA, 21 bp, Artificial sequence
  Synthetic polynucleotide
acaugagcaaggacgaaucut

SEQ ID NO: 380 - DNA, 21 bp, Artificial sequence
  Synthetic polynucleotide
uugcucauguccauguagcut

SEQ ID NO: 381 - DNA, 21 bp, Artificial sequence
  Synthetic polynucleotide
gucgcucuccccggucaggut

SEQ ID NO: 382 - DNA, 21 bp, Artificial sequence
  Synthetic polynucleotide
cgucgcucuccccggucagut

SEQ ID NO: 383 - DNA, 21 bp, Artificial sequence
  Synthetic polynucleotide
ccgucgcucuccccggucaut

SEQ ID NO: 384 - DNA, 21 bp, Artificial sequence
  Synthetic polynucleotide
accgucgcucuccccggucut

SEQ ID NO: 385 - DNA, 21 bp, Artificial sequence
  Synthetic polynucleotide
caccgucgcucuccccgguut

SEQ ID NO: 386 - DNA, 21 bp, Artificial sequence
  Synthetic polynucleotide
ccaccgucgcucuccccggut

SEQ ID NO: 387 - DNA, 21 bp, Artificial sequence
  Synthetic polynucleotide
gccaccgucgcucuccccgut

SEQ ID NO: 388 - DNA, 21 bp, Artificial sequence
  Synthetic polynucleotide
agccaccgucgcucuccccut

SEQ ID NO: 389 - DNA, 21 bp, Artificial sequence
  Synthetic polynucleotide
uagccaccgucgcucucccut

SEQ ID NO: 390 - DNA, 21 bp, Artificial sequence
  Synthetic polynucleotide
guagccaccgucgcucuccut

SEQ ID NO: 391 - DNA, 21 bp, Artificial sequence
  Synthetic polynucleotide
uguagccaccgucgcucucut

SEQ ID NO: 392 - DNA, 21 bp, Artificial sequence
  Synthetic polynucleotide
auguagccaccgucgcucuut

SEQ ID NO: 393 - DNA, 21 bp, Artificial sequence
  Synthetic polynucleotide
cauguagccaccgucgcucut

SEQ ID NO: 394 - DNA, 21 bp, Artificial sequence
  Synthetic polynucleotide
ccauguagccaccgucgcuut

SEQ ID NO: 395 - DNA, 21 bp, Artificial sequence
  Synthetic polynucleotide
uccauguagccaccgucgcut

SEQ ID NO: 396 - DNA, 21 bp, Artificial sequence
  Synthetic polynucleotide
guccauguagccaccgucgut

SEQ ID NO: 397 - DNA, 21 bp, Artificial sequence
  Synthetic polynucleotide
uguccauguagccaccgucut

SEQ ID NO: 398 - DNA, 21 bp, Artificial sequence
  Synthetic polynucleotide
auguccauguagccaccguut

SEQ ID NO: 399 - DNA, 21 bp, Artificial sequence
  Synthetic polynucleotide
cauguccauguagccaccgut

SEQ ID NO: 400 - DNA, 21 bp, Artificial sequence
  Synthetic polynucleotide
ucauguccauguagccaccut

SEQ ID NO: 401 - DNA, 21 bp, Artificial sequence
  Synthetic polynucleotide
cucauguccauguagccacut

SEQ ID NO: 402 - DNA, 21 bp, Artificial sequence
  Synthetic polynucleotide
gcucauguccauguagccaut

SEQ ID NO: 403 - DNA, 21 bp, Artificial sequence
  Synthetic polynucleotide
ugcucauguccauguagccut

SEQ ID NO: 404 - DNA, 21 bp, Artificial sequence
  Synthetic polynucleotide
cuugcucauguccauguagut

SEQ ID NO: 405 - DNA, 21 bp, Artificial sequence
  Synthetic polynucleotide
ccuugcucauguccauguaut

SEQ ID NO: 406 - DNA, 21 bp, Artificial sequence
  Synthetic polynucleotide
uccuugcucauguccauguut

SEQ ID NO: 407 - DNA, 21 bp, Artificial sequence
  Synthetic polynucleotide
guccuugcucauguccaugut

SEQ ID NO: 408 - DNA, 21 bp, Artificial sequence
  Synthetic polynucleotide
cguccuugcucauguccauut

SEQ ID NO: 409 - DNA, 21 bp, Artificial sequence
  Synthetic polynucleotide
ucguccuugcucauguccaut

SEQ ID NO: 410 - DNA, 21 bp, Artificial sequence
  Synthetic polynucleotide
uucguccuugcucauguccut

SEQ ID NO: 411 - DNA, 21 bp, Artificial sequence
  Synthetic polynucleotide
auucguccuugcucaugucut

SEQ ID NO: 412 - DNA, 21 bp, Artificial sequence
  Synthetic polynucleotide
gauucguccuugcucauguut

SEQ ID NO: 413 - DNA, 21 bp, Artificial sequence
  Synthetic polynucleotide
agcccaaugagagugacaaut

SEQ ID NO: 414 - DNA, 21 bp, Artificial sequence
  Synthetic polynucleotide
cagcuccguccucuacaccut

SEQ ID NO: 415 - DNA, 21 bp, Artificial sequence
  Synthetic polynucleotide
agcuccguccucuacaccgut

SEQ ID NO: 416 - DNA, 21 bp, Artificial sequence
  Synthetic polynucleotide
gcuccguccucuacaccgcut

SEQ ID NO: 417 - DNA, 21 bp, Artificial sequence
  Synthetic polynucleotide
cuccguccucuacaccgccut

SEQ ID NO: 418 - DNA, 21 bp, Artificial sequence
  Synthetic polynucleotide
uccguccucuacaccgccgut

SEQ ID NO: 419 - DNA, 21 bp, Artificial sequence
  Synthetic polynucleotide
ccguccucuacaccgccguut

SEQ ID NO: 420 - DNA, 21 bp, Artificial sequence
  Synthetic polynucleotide
cguccucuacaccgccgugut

SEQ ID NO: 421 - DNA, 21 bp, Artificial sequence
  Synthetic polynucleotide
guccucuacaccgccgugcut

SEQ ID NO: 422 - DNA, 21 bp, Artificial sequence
  Synthetic polynucleotide
uccucuacaccgccgugcaut

SEQ ID NO: 423 - DNA, 21 bp, Artificial sequence
  Synthetic polynucleotide
ccucuacaccgccgugcagut

SEQ ID NO: 424 - DNA, 21 bp, Artificial sequence
  Synthetic polynucleotide
cucuacaccgccgugcagcut

SEQ ID NO: 425 - DNA, 21 bp, Artificial sequence
  Synthetic polynucleotide
ucuacaccgccgugcagccut

SEQ ID NO: 426 - DNA, 21 bp, Artificial sequence
  Synthetic polynucleotide
cuacaccgccgugcagcccut

SEQ ID NO: 427 - DNA, 21 bp, Artificial sequence
  Synthetic polynucleotide
uacaccgccgugcagcccaut

SEQ ID NO: 428 - DNA, 21 bp, Artificial sequence
  Synthetic polynucleotide
acaccgccgugcagcccaaut

SEQ ID NO: 429 - DNA, 21 bp, Artificial sequence
  Synthetic polynucleotide
caccgccgugcagcccaauut

SEQ ID NO: 430 - DNA, 21 bp, Artificial sequence
  Synthetic polynucleotide
accgccgugcagcccaaugut

SEQ ID NO: 431 - DNA, 21 bp, Artificial sequence
  Synthetic polynucleotide
ccgccgugcagcccaaugaut

SEQ ID NO: 432 - DNA, 21 bp, Artificial sequence
  Synthetic polynucleotide
cgccgugcagcccaaugagut

SEQ ID NO: 433 - DNA, 21 bp, Artificial sequence
  Synthetic polynucleotide
gccgugcagcccaaugagaut

SEQ ID NO: 434 - DNA, 21 bp, Artificial sequence
  Synthetic polynucleotide
ccgugcagcccaaugagagut

SEQ ID NO: 435 - DNA, 21 bp, Artificial sequence
  Synthetic polynucleotide
cgugcagcccaaugagaguut

SEQ ID NO: 436 - DNA, 21 bp, Artificial sequence
  Synthetic polynucleotide
gugcagcccaaugagagugut

SEQ ID NO: 437 - DNA, 21 bp, Artificial sequence
  Synthetic polynucleotide
ugcagcccaaugagagugaut

SEQ ID NO: 438 - DNA, 21 bp, Artificial sequence
  Synthetic polynucleotide
gcagcccaaugagagugacut

SEQ ID NO: 439 - DNA, 21 bp, Artificial sequence
  Synthetic polynucleotide
cagcccaaugagagugacaut

SEQ ID NO: 440 - DNA, 21 bp, Artificial sequence
  Synthetic polynucleotide
gcccaaugagagugacaacut

SEQ ID NO: 441 - DNA, 21 bp, Artificial sequence
  Synthetic polynucleotide
cccaaugagagugacaacgut

SEQ ID NO: 442 - DNA, 21 bp, Artificial sequence
  Synthetic polynucleotide
ccaaugagagugacaacgaut

SEQ ID NO: 443 - DNA, 21 bp, Artificial sequence
  Synthetic polynucleotide
caaugagagugacaacgacut

SEQ ID NO: 444 - DNA, 21 bp, Artificial sequence
  Synthetic polynucleotide
aaugagagugacaacgacuut

SEQ ID NO: 445 - DNA, 21 bp, Artificial sequence
  Synthetic polynucleotide
augagagugacaacgacuaut

SEQ ID NO: 446 - DNA, 21 bp, Artificial sequence
  Synthetic polynucleotide
ugagagugacaacgacuacut

SEQ ID NO: 447 - DNA, 21 bp, Artificial sequence
  Synthetic polynucleotide
gagagugacaacgacuacaut

SEQ ID NO: 448 - DNA, 21 bp, Artificial sequence
  Synthetic polynucleotide
agagugacaacgacuacauut

SEQ ID NO: 449 - DNA, 21 bp, Artificial sequence
  Synthetic polynucleotide
gagugacaacgacuacaucut

SEQ ID NO: 450 - DNA, 21 bp, Artificial sequence
  Synthetic polynucleotide
agugacaacgacuacaucaut

SEQ ID NO: 451 - DNA, 21 bp, Artificial sequence
  Synthetic polynucleotide
gugacaacgacuacaucauut

SEQ ID NO: 452 - DNA, 21 bp, Artificial sequence
  Synthetic polynucleotide
ugacaacgacuacaucaucut

SEQ ID NO: 453 - DNA, 21 bp, Artificial sequence
  Synthetic polynucleotide
gacaacgacuacaucauccut

SEQ ID NO: 454 - DNA, 21 bp, Artificial sequence
  Synthetic polynucleotide
acaacgacuacaucaucccut

SEQ ID NO: 455 - DNA, 21 bp, Artificial sequence
  Synthetic polynucleotide
caacgacuacaucauccccut

SEQ ID NO: 456 - DNA, 21 bp, Artificial sequence
  Synthetic polynucleotide
uugucacucucauugggcuut

SEQ ID NO: 457 - DNA, 21 bp, Artificial sequence
  Synthetic polynucleotide
gguguagaggacggagcugut

SEQ ID NO: 458 - DNA, 21 bp, Artificial sequence
  Synthetic polynucleotide
cgguguagaggacggagcuut

SEQ ID NO: 459 - DNA, 21 bp, Artificial sequence
  Synthetic polynucleotide
gcgguguagaggacggagcut

SEQ ID NO: 460 - DNA, 21 bp, Artificial sequence
  Synthetic polynucleotide
ggcgguguagaggacggagut

SEQ ID NO: 461 - DNA, 21 bp, Artificial sequence
  Synthetic polynucleotide
cggcgguguagaggacggaut

SEQ ID NO: 462 - DNA, 21 bp, Artificial sequence
  Synthetic polynucleotide
acggcgguguagaggacggut

SEQ ID NO: 463 - DNA, 21 bp, Artificial sequence
  Synthetic polynucleotide
cacggcgguguagaggacgut

SEQ ID NO: 464 - DNA, 21 bp, Artificial sequence
  Synthetic polynucleotide
gcacggcgguguagaggacut

SEQ ID NO: 465 - DNA, 21 bp, Artificial sequence
  Synthetic polynucleotide
ugcacggcgguguagaggaut

SEQ ID NO: 466 - DNA, 21 bp, Artificial sequence
  Synthetic polynucleotide
cugcacggcgguguagaggut

SEQ ID NO: 467 - DNA, 21 bp, Artificial sequence
  Synthetic polynucleotide
gcugcacggcgguguagagut

SEQ ID NO: 468 - DNA, 21 bp, Artificial sequence
  Synthetic polynucleotide
ggcugcacggcgguguagaut

SEQ ID NO: 469 - DNA, 21 bp, Artificial sequence
  Synthetic polynucleotide
gggcugcacggcgguguagut

SEQ ID NO: 470 - DNA, 21 bp, Artificial sequence
  Synthetic polynucleotide
ugggcugcacggcgguguaut

SEQ ID NO: 471 - DNA, 21 bp, Artificial sequence
  Synthetic polynucleotide
uugggcugcacggcgguguut

SEQ ID NO: 472 - DNA, 21 bp, Artificial sequence
  Synthetic polynucleotide
auugggcugcacggcggugut

SEQ ID NO: 473 - DNA, 21 bp, Artificial sequence
  Synthetic polynucleotide
cauugggcugcacggcgguut

SEQ ID NO: 474 - DNA, 21 bp, Artificial sequence
  Synthetic polynucleotide
ucauugggcugcacggcggut

SEQ ID NO: 475 - DNA, 21 bp, Artificial sequence
  Synthetic polynucleotide
cucauugggcugcacggcgut

SEQ ID NO: 476 - DNA, 21 bp, Artificial sequence
  Synthetic polynucleotide
ucucauugggcugcacggcut

SEQ ID NO: 477 - DNA, 21 bp, Artificial sequence
  Synthetic polynucleotide
cucucauugggcugcacggut

SEQ ID NO: 478 - DNA, 21 bp, Artificial sequence
  Synthetic polynucleotide
acucucauugggcugcacgut

SEQ ID NO: 479 - DNA, 21 bp, Artificial sequence
  Synthetic polynucleotide
cacucucauugggcugcacut

SEQ ID NO: 480 - DNA, 21 bp, Artificial sequence
  Synthetic polynucleotide
ucacucucauugggcugcaut

SEQ ID NO: 481 - DNA, 21 bp, Artificial sequence
  Synthetic polynucleotide
gucacucucauugggcugcut

SEQ ID NO: 482 - DNA, 21 bp, Artificial sequence
  Synthetic polynucleotide
ugucacucucauugggcugut

SEQ ID NO: 483 - DNA, 21 bp, Artificial sequence
  Synthetic polynucleotide
guugucacucucauugggcut

SEQ ID NO: 484 - DNA, 21 bp, Artificial sequence
  Synthetic polynucleotide
cguugucacucucauugggut

SEQ ID NO: 485 - DNA, 21 bp, Artificial sequence
  Synthetic polynucleotide
ucguugucacucucauuggut

SEQ ID NO: 486 - DNA, 21 bp, Artificial sequence
  Synthetic polynucleotide
gucguugucacucucauugut

SEQ ID NO: 487 - DNA, 21 bp, Artificial sequence
  Synthetic polynucleotide
agucguugucacucucauuut

SEQ ID NO: 488 - DNA, 21 bp, Artificial sequence
  Synthetic polynucleotide
uagucguugucacucucauut

SEQ ID NO: 489 - DNA, 21 bp, Artificial sequence
  Synthetic polynucleotide
guagucguugucacucucaut

SEQ ID NO: 490 - DNA, 21 bp, Artificial sequence
  Synthetic polynucleotide
uguagucguugucacucucut

SEQ ID NO: 491 - DNA, 21 bp, Artificial sequence
  Synthetic polynucleotide
auguagucguugucacucuut

SEQ ID NO: 492 - DNA, 21 bp, Artificial sequence
  Synthetic polynucleotide
gauguagucguugucacucut

SEQ ID NO: 493 - DNA, 21 bp, Artificial sequence
  Synthetic polynucleotide
ugauguagucguugucacuut

SEQ ID NO: 494 - DNA, 21 bp, Artificial sequence
  Synthetic polynucleotide
augauguagucguugucacut

SEQ ID NO: 495 - DNA, 21 bp, Artificial sequence
  Synthetic polynucleotide
gaugauguagucguugucaut

SEQ ID NO: 496 - DNA, 21 bp, Artificial sequence
  Synthetic polynucleotide
ggaugauguagucguugucut

SEQ ID NO: 497 - DNA, 21 bp, Artificial sequence
  Synthetic polynucleotide
gggaugauguagucguuguut

SEQ ID NO: 498 - DNA, 21 bp, Artificial sequence
  Synthetic polynucleotide
ggggaugauguagucguugut

SEQ ID NO: 499 - DNA, 21 bp, Artificial sequence
  Synthetic polynucleotide
acccugaaugaagucaacaut

SEQ ID NO: 500 - DNA, 21 bp, Artificial sequence
  Synthetic polynucleotide
agccagcuccacccugaauut

SEQ ID NO: 501 - DNA, 21 bp, Artificial sequence
  Synthetic polynucleotide
gccagcuccacccugaaugut

SEQ ID NO: 502 - DNA, 21 bp, Artificial sequence
  Synthetic polynucleotide
ccagcuccacccugaaugaut

SEQ ID NO: 503 - DNA, 21 bp, Artificial sequence
  Synthetic polynucleotide
cagcuccacccugaaugaaut

SEQ ID NO: 504 - DNA, 21 bp, Artificial sequence
  Synthetic polynucleotide
agcuccacccugaaugaagut

SEQ ID NO: 505 - DNA, 21 bp, Artificial sequence
  Synthetic polynucleotide
gcuccacccugaaugaaguut

SEQ ID NO: 506 - DNA, 21 bp, Artificial sequence
  Synthetic polynucleotide
cuccacccugaaugaagucut

SEQ ID NO: 507 - DNA, 21 bp, Artificial sequence
  Synthetic polynucleotide
uccacccugaaugaagucaut

SEQ ID NO: 508 - DNA, 21 bp, Artificial sequence
  Synthetic polynucleotide
ccacccugaaugaagucaaut

SEQ ID NO: 509 - DNA, 21 bp, Artificial sequence
  Synthetic polynucleotide
cacccugaaugaagucaacut

SEQ ID NO: 510 - DNA, 21 bp, Artificial sequence
  Synthetic polynucleotide
cccugaaugaagucaacacut

SEQ ID NO: 511 - DNA, 21 bp, Artificial sequence
  Synthetic polynucleotide
ccugaaugaagucaacaccut

SEQ ID NO: 512 - DNA, 21 bp, Artificial sequence
  Synthetic polynucleotide
cugaaugaagucaacaccuut

SEQ ID NO: 513 - DNA, 21 bp, Artificial sequence
  Synthetic polynucleotide
ugaaugaagucaacaccucut

SEQ ID NO: 514 - DNA, 21 bp, Artificial sequence
  Synthetic polynucleotide
gaaugaagucaacaccuccut

SEQ ID NO: 515 - DNA, 21 bp, Artificial sequence
  Synthetic polynucleotide
aaugaagucaacaccuccuut

SEQ ID NO: 516 - DNA, 21 bp, Artificial sequence
  Synthetic polynucleotide
augaagucaacaccuccucut

SEQ ID NO: 517 - DNA, 21 bp, Artificial sequence
  Synthetic polynucleotide
uguugacuucauucaggguut

SEQ ID NO: 518 - DNA, 21 bp, Artificial sequence
  Synthetic polynucleotide
auucaggguggagcuggcuut

SEQ ID NO: 519 - DNA, 21 bp, Artificial sequence
  Synthetic polynucleotide
cauucaggguggagcuggcut

SEQ ID NO: 520 - DNA, 21 bp, Artificial sequence
  Synthetic polynucleotide
ucauucaggguggagcuggut

SEQ ID NO: 521 - DNA, 21 bp, Artificial sequence
  Synthetic polynucleotide
uucauucaggguggagcugut

SEQ ID NO: 522 - DNA, 21 bp, Artificial sequence
  Synthetic polynucleotide
cuucauucaggguggagcuut

SEQ ID NO: 523 - DNA, 21 bp, Artificial sequence
  Synthetic polynucleotide
acuucauucaggguggagcut

SEQ ID NO: 524 - DNA, 21 bp, Artificial sequence
  Synthetic polynucleotide
gacuucauucaggguggagut

SEQ ID NO: 525 - DNA, 21 bp, Artificial sequence
  Synthetic polynucleotide
ugacuucauucaggguggaut

SEQ ID NO: 526 - DNA, 21 bp, Artificial sequence
  Synthetic polynucleotide
uugacuucauucaggguggut

SEQ ID NO: 527 - DNA, 21 bp, Artificial sequence
  Synthetic polynucleotide
guugacuucauucagggugut

SEQ ID NO: 528 - DNA, 21 bp, Artificial sequence
  Synthetic polynucleotide
guguugacuucauucagggut

SEQ ID NO: 529 - DNA, 21 bp, Artificial sequence
  Synthetic polynucleotide
gguguugacuucauucaggut

SEQ ID NO: 530 - DNA, 21 bp, Artificial sequence
  Synthetic polynucleotide
agguguugacuucauucagut

SEQ ID NO: 531 - DNA, 21 bp, Artificial sequence
  Synthetic polynucleotide
gagguguugacuucauucaut

SEQ ID NO: 532 - DNA, 21 bp, Artificial sequence
  Synthetic polynucleotide
ggagguguugacuucauucut

SEQ ID NO: 533 - DNA, 21 bp, Artificial sequence
  Synthetic polynucleotide
aggagguguugacuucauuut

SEQ ID NO: 534 - DNA, 21 bp, Artificial sequence
  Synthetic polynucleotide
gaggagguguugacuucauut

SEQ ID NO: 535 - DNA, 21 bp, Artificial sequence
  Synthetic polynucleotide
ucaccuagguuuacaaauaut

SEQ ID NO: 536 - DNA, 21 bp, Artificial sequence
  Synthetic polynucleotide
aaucaccuagguuuacaaaut

SEQ ID NO: 537 - DNA, 21 bp, Artificial sequence
  Synthetic polynucleotide
aucaccuagguuuacaaauut

SEQ ID NO: 538 - DNA, 21 bp, Artificial sequence
  Synthetic polynucleotide
caccuagguuuacaaauacut

SEQ ID NO: 539 - DNA, 21 bp, Artificial sequence
  Synthetic polynucleotide
accuagguuuacaaauacuut

SEQ ID NO: 540 - DNA, 21 bp, Artificial sequence
  Synthetic polynucleotide
ccuagguuuacaaauacuuut

SEQ ID NO: 541 - DNA, 21 bp, Artificial sequence
  Synthetic polynucleotide
uauuuguaaaccuaggugaut

SEQ ID NO: 542 - DNA, 21 bp, Artificial sequence
  Synthetic polynucleotide
uuuguaaaccuaggugauuut

SEQ ID NO: 543 - DNA, 21 bp, Artificial sequence
  Synthetic polynucleotide
auuuguaaaccuaggugauut

SEQ ID NO: 544 - DNA, 21 bp, Artificial sequence
  Synthetic polynucleotide
guauuuguaaaccuaggugut

SEQ ID NO: 545 - DNA, 21 bp, Artificial sequence
  Synthetic polynucleotide
aguauuuguaaaccuagguut

SEQ ID NO: 546 - DNA, 21 bp, Artificial sequence
  Synthetic polynucleotide
aaguauuuguaaaccuaggut

SEQ ID NO: 547 - DNA, 21 bp, Artificial sequence
  Synthetic polynucleotide
gcaucuucaacagccucuaut

SEQ ID NO: 548 - DNA, 21 bp, Artificial sequence
  Synthetic polynucleotide
ccgacgagaucuaugagauut

SEQ ID NO: 549 - DNA, 21 bp, Artificial sequence
  Synthetic polynucleotide
uagaggcuguugaagaugcut

SEQ ID NO: 550 - DNA, 21 bp, Artificial sequence
  Synthetic polynucleotide
aucucauagaucucgucggut

SEQ ID NO: 551 - DNA, 21 bp, Artificial sequence
  Synthetic polynucleotide
ccugcucugggagaucuucut

SEQ ID NO: 552 - DNA, 21 bp, Artificial sequence
  Synthetic polynucleotide
ucugaccugcucggguucaut

SEQ ID NO: 553 - DNA, 21 bp, Artificial sequence
  Synthetic polynucleotide
gaguacaucuacguggaccut

SEQ ID NO: 554 - DNA, 21 bp, Artificial sequence
  Synthetic polynucleotide
cgagauccgauggaaggugut

SEQ ID NO: 555 - DNA, 21 bp, Artificial sequence
  Synthetic polynucleotide
agguggccaauggcauggaut

SEQ ID NO: 556 - DNA, 21 bp, Artificial sequence
  Synthetic polynucleotide
cccagcuggugcugcuucuut

SEQ ID NO: 557 - DNA, 21 bp, Artificial sequence
  Synthetic polynucleotide
aucugcaaaaccaccauugut

SEQ ID NO: 558 - DNA, 21 bp, Artificial sequence
  Synthetic polynucleotide
uacaucuuugugccagaucut

SEQ ID NO: 559 - DNA, 21 bp, Artificial sequence
  Synthetic polynucleotide
ccuuacgauaacuacguucut

SEQ ID NO: 560 - DNA, 21 bp, Artificial sequence
  Synthetic polynucleotide
uggucaacuucgaguggacut

SEQ ID NO: 561 - DNA, 21 bp, Artificial sequence
  Synthetic polynucleotide
aaguggccgucaagaugcuut

SEQ ID NO: 562 - DNA, 21 bp, Artificial sequence
  Synthetic polynucleotide
cagugccgaguuagaagacut

SEQ ID NO: 563 - DNA, 21 bp, Artificial sequence
  Synthetic polynucleotide
agccagagccccagcuugaut

SEQ ID NO: 564 - DNA, 21 bp, Artificial sequence
  Synthetic polynucleotide
cagguaguguucgaggccuut

SEQ ID NO: 565 - DNA, 21 bp, Artificial sequence
  Synthetic polynucleotide
ugacacugcacgagaagaaut

SEQ ID NO: 566 - DNA, 21 bp, Artificial sequence
  Synthetic polynucleotide
cuugaugacccagaaucuaut

SEQ ID NO: 567 - DNA, 21 bp, Artificial sequence
  Synthetic polynucleotide
gaggugguaaauuaacuuuut

SEQ ID NO: 568 - DNA, 21 bp, Artificial sequence
  Synthetic polynucleotide
cucaaggaaucauagcucuut

SEQ ID NO: 569 - DNA, 21 bp, Artificial sequence
  Synthetic polynucleotide
caguguugccucauccagaut

SEQ ID NO: 570 - DNA, 21 bp, Artificial sequence
  Synthetic polynucleotide
cugagccaaguacaggacaut

SEQ ID NO: 571 - DNA, 21 bp, Artificial sequence
  Synthetic polynucleotide
aauccauccaccagagucuut

SEQ ID NO: 572 - DNA, 21 bp, Artificial sequence
  Synthetic polynucleotide
ccgcaucugugaugagaauut

SEQ ID NO: 573 - DNA, 21 bp, Artificial sequence
  Synthetic polynucleotide
ccagcacuaacauucuagaut

SEQ ID NO: 574 - DNA, 21 bp, Artificial sequence
  Synthetic polynucleotide
caggugguugcacauuuguut

SEQ ID NO: 575 - DNA, 21 bp, Artificial sequence
  Synthetic polynucleotide
acaguuaugucuuguaaaaut

SEQ ID NO: 576 - DNA, 21 bp, Artificial sequence
  Synthetic polynucleotide
cuagcagugacaucucauuut

SEQ ID NO: 577 - DNA, 21 bp, Artificial sequence
  Synthetic polynucleotide
aauacgguaccaaagauauut

SEQ ID NO: 578 - DNA, 21 bp, Artificial sequence
  Synthetic polynucleotide
ggacucacguuaacucacaut

SEQ ID NO: 579 - DNA, 21 bp, Artificial sequence
  Synthetic polynucleotide
cucacguuaacucacauuuut

SEQ ID NO: 580 - DNA, 21 bp, Artificial sequence
  Synthetic polynucleotide
cucacauuuauacagcagaut

SEQ ID NO: 581 - DNA, 21 bp, Artificial sequence
  Synthetic polynucleotide
gaagaucucccagagcaggut

SEQ ID NO: 582 - DNA, 21 bp, Artificial sequence
  Synthetic polynucleotide
ugaacccgagcaggucagaut

SEQ ID NO: 583 - DNA, 21 bp, Artificial sequence
  Synthetic polynucleotide
gguccacguagauguacucut

SEQ ID NO: 584 - DNA, 21 bp, Artificial sequence
  Synthetic polynucleotide
caccuuccaucggaucucgut

SEQ ID NO: 585 - DNA, 21 bp, Artificial sequence
  Synthetic polynucleotide
uccaugccauuggccaccuut

SEQ ID NO: 586 - DNA, 21 bp, Artificial sequence
  Synthetic polynucleotide
agaagcagcaccagcugggut

SEQ ID NO: 587 - DNA, 21 bp, Artificial sequence
  Synthetic polynucleotide
caauggugguuuugcagauut

SEQ ID NO: 588 - DNA, 21 bp, Artificial sequence
  Synthetic polynucleotide
gaucuggcacaaagauguaut

SEQ ID NO: 589 - DNA, 21 bp, Artificial sequence
  Synthetic polynucleotide
gaacguaguuaucguaaggut

SEQ ID NO: 590 - DNA, 21 bp, Artificial sequence
  Synthetic polynucleotide
guccacucgaaguugaccaut

SEQ ID NO: 591 - DNA, 21 bp, Artificial sequence
  Synthetic polynucleotide
agcaucuugacggccacuuut

SEQ ID NO: 592 - DNA, 21 bp, Artificial sequence
  Synthetic polynucleotide
gucuucuaacucggcacugut

SEQ ID NO: 593 - DNA, 21 bp, Artificial sequence
  Synthetic polynucleotide
ucaagcuggggcucuggcuut

SEQ ID NO: 594 - DNA, 21 bp, Artificial sequence
  Synthetic polynucleotide
aggccucgaacacuaccugut

SEQ ID NO: 595 - DNA, 21 bp, Artificial sequence
  Synthetic polynucleotide
uucuucucgugcagugucaut

SEQ ID NO: 596 - DNA, 21 bp, Artificial sequence
  Synthetic polynucleotide
uagauucugggucaucaagut

SEQ ID NO: 597 - DNA, 21 bp, Artificial sequence
  Synthetic polynucleotide
aaaguuaauuuaccaccucut

SEQ ID NO: 598 - DNA, 21 bp, Artificial sequence
  Synthetic polynucleotide
agagcuaugauuccuugagut

SEQ ID NO: 599 - DNA, 21 bp, Artificial sequence
  Synthetic polynucleotide
ucuggaugaggcaacacugut

SEQ ID NO: 600 - DNA, 21 bp, Artificial sequence
  Synthetic polynucleotide
uguccuguacuuggcucagut

SEQ ID NO: 601 - DNA, 21 bp, Artificial sequence
  Synthetic polynucleotide
agacucugguggauggauuut

SEQ ID NO: 602 - DNA, 21 bp, Artificial sequence
  Synthetic polynucleotide
auucucaucacagaugcggut

SEQ ID NO: 603 - DNA, 21 bp, Artificial sequence
  Synthetic polynucleotide
ucuagaauguuagugcuggut

SEQ ID NO: 604 - DNA, 21 bp, Artificial sequence
  Synthetic polynucleotide
acaaaugugcaaccaccugut

SEQ ID NO: 605 - DNA, 21 bp, Artificial sequence
  Synthetic polynucleotide
uuuuacaagacauaacuguut

SEQ ID NO: 606 - DNA, 21 bp, Artificial sequence
  Synthetic polynucleotide
aaugagaugucacugcuagut

SEQ ID NO: 607 - DNA, 21 bp, Artificial sequence
  Synthetic polynucleotide
auaucuuugguaccguauuut

SEQ ID NO: 608 - DNA, 21 bp, Artificial sequence
  Synthetic polynucleotide
ugugaguuaacgugaguccut

SEQ ID NO: 609 - DNA, 21 bp, Artificial sequence
  Synthetic polynucleotide
aaaugugaguuaacgugagut

SEQ ID NO: 610 - DNA, 21 bp, Artificial sequence
  Synthetic polynucleotide
ucugcuguauaaaugugagut

SEQ ID NO: 611 - DNA, 23 bp, Artificial sequence
  Synthetic polynucleotide
cuccaggugucauccaucadtdt

SEQ ID NO: 612 - DNA, 23 bp, Artificial sequence
  Synthetic polynucleotide
ugauggaugacaccuggagdtdt

SEQ ID NO: 613 - DNA, 21 bp, Artificial sequence
  Synthetic polynucleotide
ucaccuagguuuacaaauaut

SEQ ID NO: 614 - DNA, 21 bp, Artificial sequence
  Synthetic polynucleotide
uauuuguaaaccuaggugaut

SEQ ID NO: 615 - DNA, 19 bp, Artificial sequence
  Synthetic polynucleotide
cucacguuaacucacauuu

SEQ ID NO: 616 - DNA, 19 bp, Artificial sequence
  Synthetic polynucleotide
aaaugugaguuaacgugag

(19) United States
(12) Patent Application Publication
(10) Pub. No.: US 2021/0292768 A1
(43) Pub. Date: Sep. 23, 2021

(54) COMPOSITIONS AND AGENTS AGAINST NONALCOHOLIC STEATOHEPATITIS

(71) Applicant: ARCTURUS THERAPEUTICS, INC., San Diego, CA (US)

(72) Inventors: Kiyoshi Tachikawa, San Diego, CA (US); Padmanabh Chivukula, San Diego, CA (US); Lily Xu, San Diego, CA (US); Angel Leu, San Diego, CA (US); Marciano Sablad, San Diego, CA (US); Rajesh Mukthavaram, San Diego, CA (US); Priya Karmali, San Diego, CA (US)

(21) Appl. No.: 17/266,556

(22) PCT Filed: Aug. 8, 2019
(86) PCT No.: PCT/US2019/045782

§ 371 Date: Feb. 5, 2021

Related U.S. Application Data
(60) Provisional application No. 62/716,004, filed on Aug. 8, 2018.

(51) Int. Cl. C12N 15/113; A61P 1/16

(52) U.S. Cl. CPC C12N 15/1138; A61P 1/16; C12N 2310/351; C12N 2310/3231; C12N 2320/32; C12N 2310/14; C12N 2310/3515

(57) ABSTRACT

This disclosure encompasses compounds and compositions useful in methods for medical therapy, in general, for inhibiting expression of PDGFRB in a subject. The compounds have a first strand and a second strand, each of the strands being 19-29 monomers in length, the monomers comprising UNA monomers and nucleic acid monomers.

### CROSS REFERENCE TO RELATED APPLICATIONS

This application claims priority under 35 U.S.C. 119(e) to U.S. Provisional Patent Application No. 62/716,004 filed on Aug. 8, 2018, the contents of which is incorporated herein by reference in its entirety.

### TECHNICAL FIELD

This disclosure herein relates to the fields of biopharmaceuticals and therapeutics composed of oligomers for gene silencing. More particularly, this disclosure relates to structures, compositions and methods for therapeutic oligomers directed against nonalcoholic steatohepatitis.

### SEQUENCE LISTING

This application includes a Sequence Listing created on Aug. 8, 2019 and submitted electronically as an ASCII file named 049386-504001WO_SLST25.txt that is 120 kilobytes and is incorporated herein in its entirety.

### BACKGROUND

Nonalcoholic fatty liver disease (NAFLD) is a condition in which excess fat is stored in the liver, but not caused by alcohol use. Nonalcoholic steatohepatitis (NASH) is a type of NAFLD. NASH is a form of NAFLD that includes hepatitis, inflammation of the liver, and liver cell damage, in addition to fat buildup in the liver. Inflammation and liver cell damage can cause fibrosis, or scarring, of the liver. NASH may lead to cirrhosis or liver cancer. About 3 to 12 percent of adults in the United States may have NASH.

No medicines have been approved to treat NASH. If NASH leads to cirrhosis, health problems caused by cirrhosis can be treated. If cirrhosis leads to liver failure, a liver transplant is possible.

Platelet-derived growth factor (PDGF) has a role in growth of smooth muscle cells, fibroblasts, and glial cells. The PDGF family has five dimeric isoforms: PDGF-AA, PDGF-BB, PDGF-CC, PDGF-DD, and PDGF-AB heterodimer. This growth factor family plays a role in embryonic development and in wound healing in adults. These growth factors mediate their effects by activating their receptor protein-tyrosine kinases, which are encoded by two genes: PDGFRA and PDGFRB. The receptors are PDGFRα/α and PDGFRβ/β homodimers, and PDGFRα/β heterodimer. PDGFRβ has a role in activating hepatic stellate cells and fibrogenesis.

What is needed are compositions and methods for treatment of NASH.

There is an urgent need for new methods and compositions for ameliorating or treating nonalcoholic steatohepatitis.

### SUMMARY

Disclosed herein are novel compounds for use as therapeutic agents against nonalcoholic steatohepatitis. The compounds of this disclosure can be used as active pharmaceutical ingredients in compositions for ameliorating, preventing or treating nonalcoholic steatohepatitis.

Embodiments of this disclosure provide a range of molecules that are useful for providing therapeutic effects because of their activity in downregulating expression of a gene. The molecules of this disclosure are structured to provide gene silencing activity in vitro and in vivo. More particularly, molecules of this disclosure are targeted for gene silencing to suppress expression of PDGFRB.

Embodiments of this disclosure can provide molecules having one or more properties that advantageously provide enhanced effectiveness against nonalcoholic steatohepatitis, as well as compositions or formulations for therapeutic agents against nonalcoholic steatohepatitis, which can provide clinical agents. The properties of the molecules of this disclosure arise according to their structure, and the molecular structure in its entirety, as a whole, can provide significant benefits and properties.

The active agents of this disclosure include oligomeric molecules that can inhibit expression of PDGFRB. Oligomers of this disclosure can provide potent action against nonalcoholic steatohepatitis in a subject by silencing expression of PDGFRB.

In some embodiments, a wide range of novel molecules are provided, which can incorporate one or more linker groups. The linker groups can be attached in a chain in the molecule. Each linker group can also be attached to a nucleobase.

In some aspects, a linker group can be a monomer. Monomers can be attached to form a chain molecule. In a chain molecule of this disclosure, a linker group monomer can be attached at any point in the chain.

In certain aspects, linker group monomers can be attached in a chain molecule of this disclosure so that the linker group monomers reside near the ends of the chain. The ends of the chain molecule can be formed by linker group monomers.

In further aspects, the linker groups of a chain molecule can each be attached to a nucleobase. The presence of nucleobases in the chain molecule can provide a sequence of nucleobases. The nucleobase sequence of an active molecule of this disclosure can be targeted with respect to a gene for suppressing expression of a gene product.

In certain embodiments, this disclosure provides oligomer molecules having chain structures that incorporate novel combinations of the linker group monomers, along with certain natural nucleotides, or non-natural nucleotides, or modified nucleotides, or chemically-modified nucleotides.

In some embodiments, the sense-antisense pairs disclosed herein comprise a LNA (Locked nucleic acid). LNAs possess a high affinity for complementary DNA and RNA sequences. Therefore, LNAs have the potential as improved therapeutic agents for repression of gene expression. Some advantages of LNAs include low toxicity, nuclease resistance and synthesis by standard methods. Examples of non-natural, modified, and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In some embodiments, a translatable molecule can contain from 1 to about 800 locked nucleic acid (LNA) monomers. In certain embodiments, a translatable molecule can contain from 1 to 12 LNA monomers, 1 to 30 LNA monomers or 1 to 100 LNA monomers.

The oligomer molecules of this disclosure can display a sequence of nucleobases that is targeted to inhibit expression of PDGFRB.

In additional aspects, this disclosure provides therapeutics for preventing, ameliorating, or treating a disease of nonalcoholic steatohepatitis. An active compound or molecule of this disclosure may be used in the prevention or treatment of nonalcoholic steatohepatitis.

This disclosure provides structures, methods and compositions for oligomeric agents that incorporate the linker group monomers. The oligomeric molecules of this disclosure can be used as active agents in formulations for gene silencing expression of PDGFRB.

In some aspects, disclosed herein is a compound comprising a first strand and a second strand, each of the strands being 19-29 monomers in length, the monomers comprising UNA monomers and nucleic acid monomers, wherein the first strand is a passenger strand for RNA interference and the second strand is a guide strand for RNA interference, and wherein the compound comprises a sequence of bases targeted for suppressing expression of PDGFRB. In some embodiments, the UNA Oligomer compound may contain one to seven UNA monomers.

The compound above, wherein the compound contains a UNA monomer at the 1-end (5′ end for non-UNA) of the first strand, a UNA monomer at the second position from the 3′ end of the first strand, and a UNA monomer at the second position from the 3′ end of the second strand.

The compound above, wherein the compound contains a UNA monomer at any one or more of positions 2 to 8 from the 5′ end of the second strand.

The compound above, wherein any one or more of the nucleic acid monomers is chemically-modified.

The compound above, wherein the compound has a 3′ overhang comprising one or more UNA monomers, natural nucleotides, non-natural nucleotides, modified nucleotides, or chemically-modified nucleotides, and combinations thereof.

The compound above, wherein the compound has a 3′ overhang comprising one or more deoxythymidine nucleotides, 2′-O-methyl nucleotides, inverted abasic monomers, inverted thymidine monomers, L-thymidine monomers, or glyceryl nucleotides.

The compound above, wherein one or more of the nucleic acid monomers is a non-natural nucleotide, a modified nucleotide, or a chemically-modified nucleotide.

The compound above, wherein each nucleic acid monomer has a 2′-O-methyl group.

The compound above, wherein the compound contains from one to eight nucleic acid monomers modified with a 2′-O-methyl group in the first strand and from one to eleven nucleic acid monomers modified with a 2′-O-methyl group in the second strand.

The compound above, wherein the compound contains one or more 2′-methoxyethoxy nucleotides, or one or more 2′-deoxy-2′-fluoro ribonucleotides.

The compound above, wherein one or more of three monomers at each end of each strand is connected by a phosphorothioate, a chiral phosphorothioate, or a phosphorodithioate linkage.

The compound above, wherein the compound has one phosphorothioate linkage between two monomers at the 1-end (5′ end) of the first strand, one phosphorothioate linkage between two monomers at the 3′ end of the first strand, one phosphorothioate linkage between monomers at the second and third positions from the 3′ end of the first strand, and one phosphorothioate linkage between two monomers at the 3′ end of the second strand.

The compound above, wherein the compound is conjugated to a delivery moiety.

The compound above, wherein the compound is conjugated to a delivery moiety that binds to a glycoprotein receptor.

The compound above, wherein the compound is conjugated to a delivery moiety that binds to a glycoprotein receptor, wherein the delivery moiety comprises a galactose, a galactosamine, or a N-acetylgalactosamine.

The compound above, wherein the compound is conjugated to a GalNAc delivery moiety.

The compound above, wherein the compound is conjugated to a cholesterol or LNA delivery moiety.

The compound above, wherein the compound is conjugated to a delivery moiety at an end of the compound and has increased uptake in the liver as compared to an unconjugated compound.

Embodiments of this disclosure further contemplate a lipid nanoparticle-oligomer compound comprising one or more compounds above attached to the lipid nanoparticle.

In further embodiments, this disclosure encompasses compositions comprising one or more compounds above and a pharmaceutically acceptable carrier. The carrier may comprise lipid nanoparticles or liposomes.

This disclosure further includes methods for preventing, ameliorating or treating a disease or condition associated with NASH in a subject in need, the method comprising administering to the subject an effective amount of the composition above. The administration of the composition may reduce inflammation of the liver, liver cell damage, liver fibrosis, or fat buildup in the liver in the subject. The subject may have been diagnosed with liver disease, or NASH.

In further aspects, this disclosure includes methods for inhibiting expression of PDGFRB in a subject in need, by administering to the subject a composition above. In some embodiments, this disclosure comprises the use of a composition for preventing, ameliorating or treating a disease or condition associated with NASH in a subject in need.

A composition of this disclosure may be used in medical therapy, or in the treatment of the human or animal body. In some embodiments, a composition of this disclosure may be used for preparing or manufacturing a medicament for preventing, ameliorating or treating a disease or condition associated with NASH in a subject in need.

This disclosure also contemplates methods for inhibiting expression of PDGFRB in a subject in need, by administering to the subject a composition above, as well as the use of a composition above for preventing, ameliorating or treating a disease or condition associated with nonalcoholic steatohepatitis in a subject in need.

In some aspects, this disclosure includes compositions for use in medical therapy, or for use in the treatment of the human or animal body. In certain aspects, this disclosure includes the use of a composition for preparing or manufacturing a medicament for preventing, ameliorating or treating a disease or condition associated with nonalcoholic steatohepatitis in a subject in need.

Additional aspects of this disclosure can include an siRNA comprising sense and antisense strands of 19-21 nucleotides, wherein the siRNA is targeted to PDGFRB.

### BRIEF DESCRIPTION OF THE DRAWINGS

FIG. 1 shows a gene map of a PDGFRB coding region and reference positions for selected therapeutic oligomer structures.

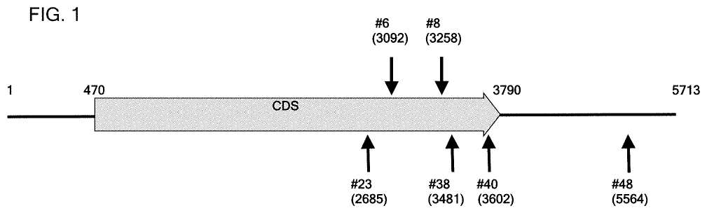

FIG. 2 shows relative PDGFRB gene expression knockdown in rat primary hepatic stellate cells (RHSteC) for selected UNA Oligomers based on structure #48 (Ref Pos 5564). Oligomer structures 1 (SEQ ID NO:103/104), 3 (SEQ ID NO:107/108), and 5 (SEQ ID NO:111/112) showed surprisingly superior PDGFRB knockdown as compared to a conventional siRNA based on the same reference position.

FIG. 3 shows relative PDGFRB gene expression knockdown in human hepatic stellate cells (LX-2) for selected UNA Oligomers based on structure #48 (Ref Pos 5564). Oligomer structures A (SEQ ID NO:111/112), B (SEQ ID NO:103/104), and C (SEQ ID NO:107/108) showed superior PDGFRB knockdown.

FIG. 4 shows relative PDGFRA gene expression knockdown in human hepatic stellate cells (LX-2) for selected UNA Oligomers based on structure #48 (Ref Pos 5564). As compared to FIG. 3, Oligomer structures A (SEQ ID NO:111/112), B (SEQ ID NO:103/104), and C (SEQ ID NO:107/108) did not substantially knockdown PDGFRA gene expression. Thus, the UNA Oligomers were surprisingly selective for reducing gene expression of PDGFRB over that of PDGFRA.

FIG. 5 shows an IL-8 assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced IL-8 stimulation as compared to a conventional siRNA based on the same reference position.

FIG. 6 shows an IL-8 assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced IL-8 stimulation as compared to a conventional siRNA based on the same reference position.

FIG. 7 shows a TNFa assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced TNFa stimulation as compared to a conventional siRNA based on the same reference position.

FIG. 8 shows an TNFa assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced TNFa stimulation as compared to a conventional siRNA based on the same reference position.

FIG. 9 shows relative PDGFRB gene expression knockdown in MDR2 knockout mice in vivo for a UNA Oligomer based on structure #48 (Ref Pos 5564). Oligomer B (SEQ ID NO:103/104) was formulated in a lipid nanoparticle formulation and administered up to 3 mg/kg.

FIG. 10 shows relative PDGFRB gene expression knockdown in human hepatic stellate cells (LX-2) for selected UNA Oligomers. Oligomer structures hcyn22 (Ref Pos 4594) (SEQ ID NO:572/602), hcyn23 (Ref Pos 4776) (SEQ ID NO:573/603), hcyn27 (Ref Pos 5545) (SEQ ID NO:577/607), and hcyn29 (Ref Pos 5594) (SEQ ID NO:579/609) showed superior PDGFRB knockdown as compared to Oligomer B (SEQ ID NO:103/104). Thus, the hcyn Oligomers are cross reactive in human and cynomolgus monkey.

FIG. 11 shows relative PDGFRB gene expression knockdown in human hepatic stellate cells (LX-2) 24 hr post transfection for selected siRNAs based on sequences #6 (Ref Pos 3092), #8 (Ref Pos 3258), #23 (Ref Pos 2685), #38 (Ref Pos 3481), #40 (Ref Pos 3602), and #48 (Ref Pos 5564). These siRNAs contained only natural nucleotides and showed useful PDGFRB knockdown.

FIG. 12 shows relative PDGFRB gene expression knockdown in human hepatic stellate cells (LX-2) for selected LNA-containing UNA Oligomers. Oligomer LNA-containing structures LNAsi-7 (Ref Pos 5564) (SEQ ID NO:335/614) and LNAsi-9 (Ref Pos 5564) (SEQ ID NO:613/614), and hcyn-29-CM1 (Ref Pos 5594) (SEQ ID NO:579/609) showed a relative Fold change of PDGFRB expression knockdown as compared to PRb48-1-CM1 (Ref Pos 5564) (SEQ ID NO:335/341).

FIG. 13 shows relative LDH cytotoxicity in human hepatic stellate cells (LX-2) for selected LNA-containing UNA Oligomers. Oligomer LNA-containing structures LNAsi-7 (Ref Pos 5564) (SEQ ID NO:335/614) and LNAsi-9 (Ref Pos 5564) (SEQ ID NO:613/614) showed superior cytotoxicity as compared to PRb48-1-CM1 (Ref Pos 5564) (SEQ ID NO:335/341).

FIG. 14 shows relative cell viability in human hepatic stellate cells (LX-2) for selected LNA-containing UNA Oligomers. Oligomer LNA-containing structures LNAsi-7 (Ref Pos 5564) (SEQ ID NO:335/614) and LNAsi-9 (Ref Pos 5564) (SEQ ID NO:613/614) showed superior cell viability as compared to PRb48-1-CM1 (Ref Pos 5564) (SEQ ID NO:335/341).

### DETAILED DESCRIPTION

This disclosure provides a range of novel agents and compositions to be used as therapeutics against nonalcoholic steatohepatitis. Molecules of this disclosure can be used as active pharmaceutical ingredients in compositions for ameliorating, preventing or treating nonalcoholic steatohepatitis.

The major feature in Nonalcoholic Fatty Liver Disease (NAFLD) is fat accumulation in hepatocytes with minimal inflammation. These patients are usually identified on the basis of a liver biopsy performed because of mildly elevated liver transaminase levels in the serum or the suspicion of fatty liver on non-invasive testing such as computerized tomography or ultrasound.

A subset of individuals with NAFLD are found to have Nonalcoholic Steatohepatitis (NASH) which is fatty liver with the addition of the development of infiltration of inflammatory cells (including but not limited to neutrophils or lymphocytes) within the lobule, central vein and portal areas and evidence of damage to hepatocytes including but not limited to ballooning degeneration. This inflammatory state of NASH may result in the deposition of fibrous tissue, including but not limited to collagen, which can lead to cirrhosis, nodule formation, and eventually hepatocellular carcinoma.

The disease progress is insidious since most people with NASH feel well and are not aware that they have a liver problem. Despite the lack of symptoms, NASH can be severe and can lead to the deposition of fibrotic material in the liver which can result in severe scarring and/or cirrhosis and, in some cases, hepatocellular carcinoma. Therefore, there is a need for clinical tests that could identify NASH early and follow its progression.

NAFLD and NASH are common disorders. It is reported by the U.S. National Institutes of Health that 10-20 percent of Americans have NAFLD and 3-5 percent have NASH. Both are becoming more common because of the greater numbers of people with obesity and diabetes, including children and adolescents. The fact that NASH can progress to cirrhosis makes this a major health problem.

Although NASH has become more common, its underlying cause is still not clear. It most often occurs in middle-aged persons who overweight or obese, many of whom have metabolic syndrome, insulin resistance, or overt diabetes. However, NASH is not simply obesity that affects the liver. NASH can affect children and adolescents.

The proximal cause of liver injury in NASH is not known. Multiple theories have been proposed, with some experimental data to suggest their involvement. Some of these include, but are not limited to, hepatocyte resistance to the action of insulin, production of inflammatory cytokines by fat cells and other inflammatory cells that damage the liver and recruit additional inflammatory cells and oxidative stress in hepatocytes with production of reactive oxygen radicals that damage liver cells and induce inflammation.

Currently, no specific therapies for NASH exist and only general health recommendations are currently provided to patients. These include weight reduction, eating a balanced and healthy diet, increasing physical activity, and avoidance of alcohol and unnecessary medications. Weight loss can improve serum liver tests in some patients with NASH and may improve evidence of histological liver damage, but it does not reverse severe liver disease and not all patients with NASH are overweight.

A variety of experimental approaches have been evaluated or are under evaluation in patients with NASH including the use of antioxidants, such as vitamin E, selenium, betaine, and anti-diabetic agents including metformin, rosiglitazone, and pioglitazone. All clinical results to date have been disappointing.

In one embodiment, disclosed herein is a compound comprising a first strand and a second strand, each of the strands being 19-29 monomers in length, the monomers comprising UNA monomers and nucleic acid monomers, wherein the first strand is a passenger strand for RNA interference and the second strand is a guide strand for RNA interference, and wherein the compound comprises at least one of the following sense-antisense pairs:

(#48) SEQ ID NO: 335 and 341;

(#48) SEQ ID NO: 336 and 342;

(#48) SEQ ID NO: 337 and 343;

(#48) SEQ ID NO: 338 and 344;

(#48) SEQ ID NO: 339 and 345;

(#48) SEQ ID NO: 340 and 346;

(LNAsi-7) SEQ ID NO: 335 and 614;

(LNAsi-9) SEQ ID NO: 613 and 614; and

(hcyn-29-CM1) SEQ ID NO: 579 and 609.

In some embodiments, any one or more of the nucleic acid monomers is chemically-modified.

In some embodiments, the compound is conjugated to a delivery moiety.

In some embodiments, the compound is conjugated to a delivery moiety that binds to a glycoprotein receptor.

In some embodiments, the compound is conjugated to a delivery moiety that binds to a glycoprotein receptor, wherein the delivery moiety comprises a galactose, a galactosamine, or a N-acetylgalactosamine.

In some embodiments, the compound is conjugated to a GalNAc delivery moiety.

In some embodiments, the compound is conjugated to a cholesterol or LNA delivery moiety.

In some embodiments, the compound is conjugated to a delivery moiety at an end of the compound and has increased uptake in the liver as compared to an unconjugated compound.

In some embodiments, the compound further comprises a lipid nanoparticle.

In another embodiment, disclosed herein is a pharmaceutical composition comprising one or more compounds as disclosed herein and a pharmaceutically acceptable carrier.

In some embodiments, the pharmaceutical composition comprises a lipid formulation; and/or one or more lipids selected from cationic lipids, anionic lipids, sterols, pegylated lipids, and any combination of the foregoing.

In some embodiments, the carrier comprises lipid nanoparticles or liposomes.

In yet another embodiment, disclosed herein is a method for treating non-alcoholic steatohepatitis in a subject, the method comprising administering to the subject an effective amount of a pharmaceutical composition comprising one or more compounds as disclosed herein and a pharmaceutically acceptable carrier.

In some embodiments, the method for treating non-alcoholic steatohepatitis in a subject in need, the method comprising inhibiting expression of PDGFRB in a subject in need, the method comprising administering to the subject a pharmaceutical composition comprising one or more compounds as disclosed herein and a pharmaceutically acceptable carrier.

In some embodiments, the method for treating non-alcoholic steatohepatitis in a subject, further comprises preventing, ameliorating or treating a disease or condition associated with NASH in a subject.

In some embodiments, the administration of the composition reduces liver size or liver steatosis.

In some embodiments, the reduction in liver size or liver steatosis is measured by biopsy or by a non-invasive method.

In one embodiment, the compounds described here are useful for human NASH as a method of ameliorating or reversing hepatocyte fat accumulation, intra-portal and intra-lobular inflammatory infiltrate, and fibrosis, including but not limited to collagen deposition in the peri-sinusoidal space, cirrhosis, and for preventing progression to hepatocellular carcinoma. Moreover, it is proposed that these improvements in liver disease pathology will have a resultant positive effect on the health of the individuals by reducing complications of liver fibrosis and cirrhosis, including the development of hepatocellular carcinoma.

In another embodiment, a therapeutically effective dose can be evaluated by a change of at least 10% in the level of the serum biomarkers of NASH. In some embodiments, the serum biomarkers of NASH can include but not limited to hyaluronic acid and other breakdown products of collagens, cytokeratin-18 and other cytoskeletal cellular proteins, tissue inhibitor of metalloprotease I and II and other liver derived collagen and matrix proteases. These compounds and biomarkers may be measured in the serum or in the liver tissue using immunoassays and the levels can be correlated with severity of disease and treatment.

In another embodiment, a therapeutically effective dose can be evaluated by a change of at least 10% in the level of the serum biomarkers of NASH including but not limited to reactive oxygen products of lipid or protein origin, coenzyme Q reduced or oxidized forms, and lipid molecules or conjugates. These biomarkers can be measured by various means including immunoassays and electrophoresis and their levels can be correlated with severity of disease and treatment.

In another embodiment, a therapeutically effective dose can be evaluated by a change of at least 10% in the level of the serum biomarkers of NASH including but not limited to cytokines that include but are not limited to TNF-alpha, TGF-beta or IL-8, osteopontin, or a metabolic profile of serum components that is indicative of NASH presence or severity (these include serum and urine markers). A profile of one or more of these cytokines, as measured by immunoassay or proteomic assessment by LC mass spec, may provide an assessment of activity of the disease and a marker to follow in therapy of the disease.

In another embodiment, a therapeutically effective dose can be evaluated by a change of at least 10% in the pathophysiologic spectrum of NASH which includes histopathological findings on liver biopsy. Histopathological findings on liver biopsy can include but are not limited to evidence of intra-hepatocellular fat, hepatocellular toxicity including but not limited to hyaline bodies, inflammatory cell infiltrates (including but not limited to lymphocytes and various subsets of lymphocytes and neutrophils), changes in bile duct cells, changes in endothelial cells, number of Kupffer cell macrophages, collagen deposition (including but not limited to pen-sinusoidal, portal and central collagen deposition and portal to central bridging collagen deposition, hepatocellular nodules that distort the normal architecture, hepatocellular atypia consistent with malignant transformation, and various scales and methods that combine various sets of observations for grading the severity of NASH. Such histological assessments are the sine-qua-non with NASH diagnosis and therefore integrally related to assessment of therapy.

In another embodiment, a therapeutically effective dose can be evaluated by a change of at least 10% in the clinical manifestations of NASH including but not limited to clinical testing of stage and severity of the disease, clinical signs and symptoms of disease, and medical complications. Clinical testing of stage and severity of NASH include but are not limited to hematologic testing (including but not limited to red blood cell count and morphology, white blood cell count and differential and morphology, platelet count and morphology), serum or plasma lipids including but not limited to triglycerides, cholesterol, fatty acids, lipoprotein species and lipid peroxidation species, serum or plasma enzymes (including but not limited to aspartate transaminase (AST), alanine transaminase (ALT), alkaline phosphatase (AP), gamma glutamyltranspeptidase (GGTP), lactate dehydrogenase (LDH) and isoforms, serum or plasma albumin and other proteins indicative of liver synthetic capacity, serum or plasma levels of bilirubin or other compounds indicative of the ability of the liver to clear metabolic byproducts, serum or plasma electrolytes (including but not limited to sodium, potassium, chloride, calcium, phosphorous), coagulation profile including but not limited to prothrombin time (PT), partial thromoplastin time (PTT), specific coagulation factor levels, bleeding time and platelet function. Clinical testing also includes but is not limited to non-invasive and invasive testing that assesses the architecture, structural integrity or function of the liver including but not limited to computerized tomography (CT scan), ultrasound (US), ultrasonic elastography (including but not limited to FibroScan) or other measurements of the elasticity of liver tissue, magnetic resonance scanning or spectroscopy, percutaneous or skinny needle or transjugular liver biopsy and histological assessment (including but not limited to staining for different components using affinity dyes or immunohistochemistry), measurement of hepatic portal-venous wedge pressure gradient, or other non-invasive or invasive tests that may be developed for assessing severity of NASH in the liver tissue.

In another embodiment, a therapeutically effective dose can be evaluated by a change of at least 10% in clinical signs and symptoms of disease include fatigue, muscle weight loss, spider angiomata, abdominal pain, abdominal swelling, ascites, gastrointestinal bleeding, other bleeding complications, easy bruising and ecchymoses, peripheral edema, hepatomegaly, nodular firm liver, somnolence, sleep disturbance, and coma. Medical complications of NASH are related to cirrhosis and include ascites, peripheral edema, esophageal and other gastrointestinal tract varices, gastrointestinal bleeding, other bleeding complications, emaciation and muscle wasting, hepatorenal syndrome, and hepatic encephalopathy. An additional complication of NASH related cirrhosis is the development of complications sufficiently severe to warrant placement on liver transplantation list or receiving a liver transplantation.

In another embodiment, a therapeutically effective dose has an effect on NASH liver disease and/or fibrosis in the absence of any effect on whole blood glucose in patients with diabetes or serum lipids in patients with elevated serum lipids.

Novel agents of this disclosure include oligomeric molecules that inhibit expression of PDGFRB.

Embodiments of this disclosure can provide extraordinary and surprisingly enhanced efficacy against nonalcoholic steatohepatitis in a subject by suppressing expression of PDGFRB.

The properties of the compounds of this disclosure arise according to their molecular structure, and the structure of the molecule in its entirety, as a whole, can provide significant benefits based on those properties. Embodiments of this disclosure can provide molecules having one or more properties that advantageously provide enhanced effectiveness against nonalcoholic steatohepatitis, as well as compositions or formulations for therapeutic agents against nonalcoholic steatohepatitis, which can provide clinical agents.

A wide range of novel molecules are provided, each of which can incorporate specialized linker groups. The linker groups can be attached in a chain in the molecule. Each linker group can also be attached to a nucleobase.

In some aspects, a linker group can be a monomer. Monomers can be attached to form a chain molecule. In a chain molecule of this disclosure, a linker group monomer can be attached at any point in the chain.

In certain aspects, linker group monomers can be attached in a chain molecule of this disclosure so that the linker group monomers reside near the ends of the chain. The ends of the chain molecule can be formed by linker group monomers.

As used herein, a chain molecule can also be referred to as an oligomer.

In further aspects, the linker groups of a chain molecule can each be attached to a nucleobase. The presence of nucleobases in the chain molecule can provide a sequence of nucleobases.

In certain embodiments, this disclosure provides oligomer molecules having chain structures that incorporate novel combinations of the linker group monomers, along with certain natural nucleotides, or non-natural nucleotides, or modified nucleotides, or chemically-modified nucleotides.

The oligomer molecules of this disclosure can display a sequence of nucleobases that is targeted for gene silencing to suppress expression of PDGFRB.

In some embodiments, an oligomer molecule of this disclosure can display a sequence of nucleobases that is targeted to a coding or non-coding region of a PDGFRB gene for suppressing expression of PDGFRB.

In some aspects, this disclosure provides active oligomer molecules that are targeted to at least a fragment of a PDGFRB nucleic acid molecule, and that decrease expression of at least such a fragment present in a cell. In some embodiments, the active oligomer molecule can be double-stranded.

In further aspects, this disclosure provides active oligomer molecules that are complementary to at least a fragment of a PDGFRB nucleic acid molecule, and that decrease expression of at least such a fragment present in a cell. In some embodiments, the active oligomer molecule can be double-stranded.

Without wishing to be bound by any one particular theory, a cellular pathway may use active oligomers of this disclosure to be sequence-specific regulators in an RNA interference pathway. The active oligomers may bind to the RNA-induced silencing complex (RISC complex), where a sense strand, also referred to as the passenger strand, and an antisense strand, also referred to as the guide strand, can be unwound, and the antisense strand complexed in the RISC complex. The guide strand can bind to a complementary sequence to which it was targeted, for example, a target sequence in an mRNA, which can be subsequently cleaved, resulting in inactivation of the nucleic acid molecule containing the target sequence. As a result, the expression of mRNA containing the target sequence can be reduced.

In some embodiments, an oligomeric molecule may be attached to a delivery moiety. Examples of delivery moieties include glycoprotein receptors, galactoses, galactosamines, N-acetylgalactosamines, and GalNAc groups.

Examples of delivery moieties include cholesterols, sterols, phytosterols, steroids, zoosterols, lanosterols, stigmastanols, dihydrolanosterols, zymosterols, zymostenols, desmosterols, and 7-dehydrocholesterols.

Examples of delivery moieties include branched and unbranched, substituted and unsubstituted C₁₂-C₂₂ alkanoyl groups and alkenoyl groups.

Examples of delivery moieties include mono-, di- and trimeric galactosyl or N-acetylamino galactosyl moieties. A galactosyl group may have one or more ring structures.

### Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N Y. Acad. Sci., 1992, 660, 306-309; Manoharan et al., Bioorg. Med. Chem. Lett., 1993, 3, 2765-2770), a thiochole sterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBSLett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster {e.g., WO2014/179620).

### Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (<S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieities, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted Ci-Cio alkyl, substituted or unsubstituted C₂-Ci₀ alkenyl or substituted or unsubstituted C₂-Ci₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue.

Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### Certain Cell-Targeting Conjugate Moieties

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, a-D-galactosamine, (3-muramic acid, 2-deoxy-2-methylamino-L-glucopyranose, 4,6-dideoxy-4-formamido-2,3-di-0-methyl-D-mannopyranose, 2-deoxy-2-sulfoamino-D-glucopyranose and N-sulfo-D-glucosamine, and N-glycoloyl-a-neuraminic acid. For example, thio sugars may be selected from 5-Thio- -D-glucopyranose, methyl 2,3,4-tri-0-acetyl-1-thio-6-0-trityl-a-D-glucopyranoside, 4-\hat{t}l\acute{η}o-β-0-galactopyranose, and ethyl 3,4,6,7-tetra-0-acetyl-2-deoxy-1,5-dithio-a-D-g/Mco-heptopyranoside.

Representative United States patents, United States patent application publications, international patent application publications, and other publications that teach the preparation of certain of the above noted conjugate groups, oligomeric compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 79, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770.

In certain embodiments, oligomeric compounds comprise modified oligonucleotides comprising a gapmer or fully modified sugar motif and a conjugate group comprising at least one, two, or three GalNAc ligands. In certain embodiments antisense compounds and oligomeric compounds comprise a conjugate group found in any of the following references: Lee, Carbohydr Res, 1978, 67, 509-514; Connolly et al., J Biol Chem, 1982, 257, 939-945; Pavia et al., Int JP ep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al, Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vase Biol, 2006, 26, 169-175; van Rossenberg et al, Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344, 125; 8,313,772; 8,349,308; 8,450,467; 8,501,930; 8,158,601; 7,262, 177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; U52010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In additional aspects, this disclosure provides therapeutics for preventing, ameliorating, or treating nonalcoholic steatohepatitis. An active compound or molecule of this disclosure may be used in the prevention or treatment of nonalcoholic steatohepatitis.

This disclosure provides structures, methods and compositions for oligomeric agents that incorporate the linker group monomers. The oligomeric molecules of this disclosure can be used as active agents in formulations for gene silencing therapeutics targeted to a PDGFRB nucleic acid molecule.

This disclosure provides a range of molecules that are useful for providing therapeutic effects because of their activity in regulating expression of a gene. The molecules of this disclosure are structured to provide gene regulating or silencing activity in vitro and in vivo.

Embodiments of this disclosure can provide molecules for use as therapeutic agents against nonalcoholic steatohepatitis. The molecules can be used as active pharmaceutical ingredients in compositions for ameliorating, preventing or treating nonalcoholic steatohepatitis.

In certain embodiments, an active molecule can be structured as an oligomer composed of monomers. The oligomeric structures of this disclosure may contain one or more linker group monomers, along with certain nucleotides.

### UNA Monomers

In some embodiments, linker group monomers can be unlocked nucleomonomers (UNA monomers), which are small organic molecules based on a propane-1,2,3-tri-yl-trisoxy structure as shown below:

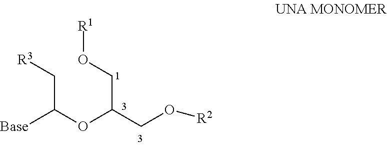

where R¹ and R² are H, and R¹ and R² can be phosphodiester linkages, Base can be a nucleobase, and R³ is a functional group described below.

In another view, the UNA monomer main atoms can be drawn in IUPAC notation as follows:

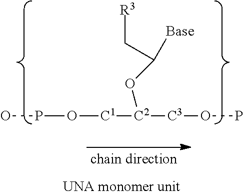

where the direction of progress of the oligomer chain is from the 1-end to the 3-end of the propane residue.

Examples of a nucleobase include uracil, thymine, cytosine, 5-methylcytosine, adenine, guanine, inosine, and natural and non-natural nucleobase analogues.

In general, because the UNA monomers are not nucleotides, they can exhibit at least four forms in an oligomer. First, a UNA monomer can be an internal monomer in an oligomer, where the UNA monomer is flanked by other monomers on both sides. In this form, the UNA monomer can participate in base pairing when the oligomer is a duplex, for example, and there are other monomers with nucleobases in the duplex.

Examples of UNA monomer as internal monomers flanked at both the propane-1-yl position and the propane-3-yl position, where R³ is —OH, are shown below.

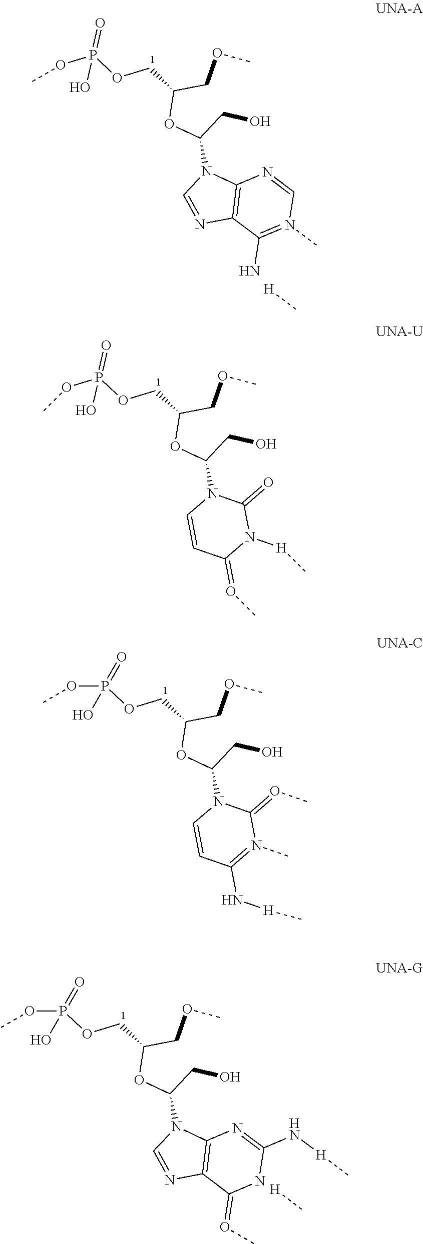

Second, a UNA monomer can be a monomer in an overhang of an oligomer duplex, where the UNA monomer is flanked by other monomers on both sides. In this form, the UNA monomer does not participate in base pairing. Because the UNA monomers are flexible organic structures, unlike nucleotides, the overhang containing a UNA monomer will be a flexible terminator for the oligomer.

A UNA monomer can be a terminal monomer in an overhang of an oligomer, where the UNA monomer is attached to only one monomer at either the propane-1-yl position or the propane-3-yl position. In this form, the UNA monomer does not participate in base pairing. Because the UNA monomers are flexible organic structures, unlike nucleotides, the overhang containing a UNA monomer can be a flexible terminator for the oligomer.

Examples of a UNA monomer as a terminal monomer attached at the propane-3-yl position are shown below.

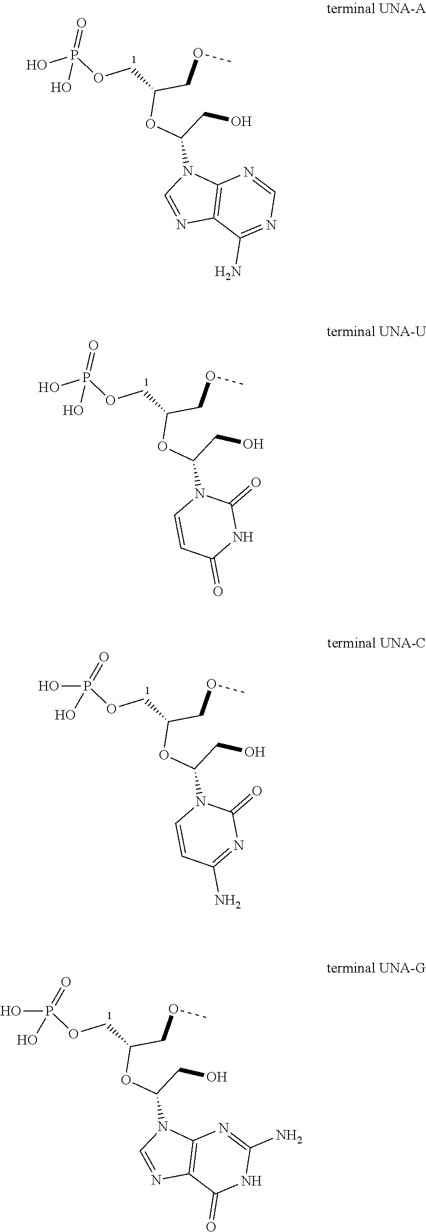

Because a UNA monomer can be a flexible molecule, a UNA monomer as a terminal monomer can assume widely differing conformations. An example of an energy minimized UNA monomer conformation as a terminal monomer attached at the propane-3-yl position is shown below.

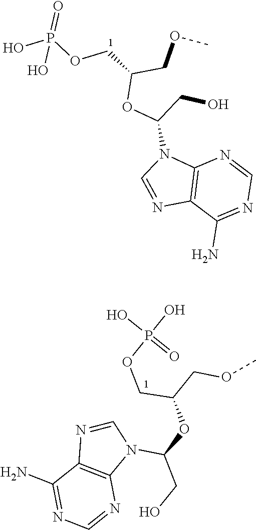

### UNA-A Terminal Forms: The Dashed Bond Shows the Propane-3-Yl Attachment

Thus, UNA oligomers having a terminal UNA monomer are significantly different in structure from conventional nucleic acid agents, such as siRNAs. For example, siRNAs may require that terminal monomers or overhangs in a duplex be stabilized. In contrast, the conformability of a terminal UNA monomer can provide UNA oligomers with different properties.

Among other things, the structure of the UNA monomer allows it to be attached to naturally-occurring nucleotides. A UNA oligomer can be a chain composed of UNA monomers, as well as various nucleotides that may be based on naturally-occurring nucleosides.

In some embodiments, the functional group R³ of a UNA monomer can be —OR⁴, —SR⁴, —NR⁴₂, —NH(C═O)R⁴, morpholino, morpholin-1-yl, piperazin-1-yl, or 4-alkanoyl-piperazin-1-yl, where R⁴ is the same or different for each occurrence, and can be H, alkyl, a cholesterol, a lipid molecule, a polyamine, an amino acid, or a polypeptide.

The UNA monomers are organic molecules. UNA monomers are not nucleic acid monomers or nucleotides, nor are they naturally-occurring nucleosides or modified naturally-occurring nucleosides.

A UNA oligomer of this disclosure is a synthetic chain molecule. A UNA oligomer of this disclosure is not a nucleic acid, nor an oligonucleotide.

### Additional Monomers for Oligomeric Agents

As used herein, in the context of oligomer sequences, the symbol X represents a UNA monomer.

As used herein, in the context of oligomer sequences, the symbol N represents any natural nucleotide monomer, or a modified nucleotide monomer.

As used herein, in the context of oligomer sequences, the symbol Q represents a non-natural, modified, or chemically-modified nucleotide monomer. When a Q monomer appears in one strand of the oligomer, and is unpaired with the other strand, the monomer can have any base attached. When a Q monomer appears in one strand of the oligomer and is paired with a monomer in the other strand, the Q monomer can have any base attached that would be complementary to the monomer in the corresponding paired position in the other strand.

Examples of nucleic acid monomers include non-natural, modified, and chemically-modified nucleotides, including any such nucleotides known in the art.

Examples of non-natural, modified, and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of non-natural, modified, and chemically-modified nucleotide monomers include 3′-end stabilized nucleotides, 3′-glyceryl nucleotides, 3′-inverted abasic nucleotides, 3′-inverted thymidine, and L-thymidine.

Examples of non-natural, modified, and chemically-modified nucleotide monomers include locked nucleic acid nucleotides, 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides.

Examples of non-natural, modified, and chemically-modified nucleotide monomers include 2′-amino nucleotides, 2′-O-amino nucleotides, 2′-C-allyl nucleotides, and 2′-O-allyl nucleotides.

Examples of non-natural, modified, and chemically-modified nucleotide monomers include N⁶-methyladenosine nucleotides.

Examples of non-natural, modified, and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of non-natural, modified, and chemically-modified nucleotide monomers include 2′-O-aminopropyl substituted nucleotides.

Examples of non-natural, modified, and chemically-modified nucleotide monomers include 2′-O-guanidinopropyl substituted nucleotides.

Examples of non-natural, modified, and chemically-modified nucleotide monomers include Pseudouridines.

Examples of non-natural, modified, and chemically-modified nucleotide monomers include replacing the 2′-OH group of a nucleotide with a 2′-R, a 2′-OR, a 2′-halogen, a 2′-SR, or a 2′-amino, 2′-azido, where R can be H, alkyl, fluorine-substituted alkyl, alkenyl, or alkynyl.

Examples of non-natural, modified, and chemically-modified nucleotide monomers include replacing the 2′-OH group of a nucleotide with a 2′-R or 2′-OR, where R can be CN, CF₃, alkylamino, or aralkyl.

Examples of non-natural, modified, and chemically-modified nucleotide monomers include nucleotides with a modified sugar such as an F-HNA, an HNA, a CeNA, a bicyclic sugar, or an LNA.

Examples of non-natural, modified, and chemically-modified nucleotide monomers include 2′-oxa-3′-aza-4′a-carbanucleoside monomers, 3-hydroxymethyl-5-(1H-1,2,3-triazol)-isoxazolidine monomers, and 5′-triazolyl-2′-oxa-3′-aza-4′a-carbanucleoside monomers.

Some examples of modified nucleotides are given in Saenger, Principles of Nucleic Acid Structure, Springer-Verlag, 1984.

### Oligomeric Compounds Containing UNA Monomers

Aspects of this disclosure can provide structures and compositions for UNA-containing oligomeric compounds. The oligomeric agents may incorporate one or more UNA monomers. Oligomeric molecules of this disclosure can be used as active agents in formulations for gene regulating or gene silencing therapeutics.

In some embodiments, this disclosure provides oligomeric compounds having a structure that incorporates novel combinations of UNA monomers with certain natural nucleotides, non-natural nucleotides, modified nucleotides, or chemically-modified nucleotides.

In further aspects, the oligomeric compounds can be pharmacologically active molecules. UNA oligomers of this disclosure can be used as active pharmaceutical ingredients for regulating gene expression, and in RNA interference methods, as well as antisense, RNA blocking, and micro-RNA strategies.

A UNA oligomer of this disclosure can have the structure of Formula I

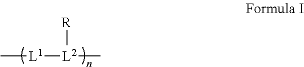

wherein L¹ is a linkage, n is from 19 to 29, and for each occurrence L² is a UNA linker group having the formula where R is attached to C² and has the formula
—OCH(CH₂R³)R⁵, where R³ is —OR⁴, —SR⁴, —NR⁴₂, —NH(C═O)R⁴, morpholino, morpholin-1-yl, piperazin-1-yl, or 4-alkanoyl-piperazin-1-yl, where R⁴ is the same or different for each occurrence and is H, alkyl, a cholesterol, a lipid molecule, a polyamine, an amino acid, or a polypeptide, and where R⁵ is a nucleobase, or L²(R) is a sugar such as a ribose and R is a nucleobase, or L² is a modified sugar such as a modified ribose and R is a nucleobase. In certain embodiments, a nucleobase can be a modified nucleobase. L¹ can be a phosphodiester linkage.

A UNA oligomer of this disclosure can be a short chain molecule. A UNA oligomer can be a duplex pair. Thus, a UNA oligomer can have a first strand of the duplex and a second strand of the duplex, which is complementary to the first strand with respect to the nucleobases, although up to three mismatches can occur. A UNA oligomer duplex can have overhangs.

Some UNA oligomers are discussed in U.S. Pat. No. 8,314,227, as well as US Patent Publication No. 20110313020 A1.

The target of a UNA oligomer can be a target nucleic acid. In some embodiments, the target can be any mRNA of a subject. A UNA oligomer can be active for gene silencing in RNA interference.

A UNA oligomer may comprise two strands that together provide a duplex. The duplex may be composed of a first strand, which may also be referred to as a passenger strand or sense strand, and a second strand, which may also be referred to as a guide strand or antisense strand.

In some aspects, a UNA oligomer of this disclosure can have any number of phosphorothioate intermonomer linkages in any position in any strand, or in both strands of a duplex structure.

In some embodiments, any one or more of the intermonomer linkages of a UNA oligomer can be a phosphodiester, a phosphorothioate including dithioates, a chiral phosphorothioate, and other chemically modified forms.

Examples of UNA oligomers of this disclosure include duplex pairs, which are in general complementary. Thus, for example, SEQ ID NO:1 can represent a first strand of a duplex and SEQ ID NO:2 can represent a second strand of the duplex, which is complementary to the first strand.

For example, the symbol “N” in the first strand can represent any nucleotide that is complementary to the monomer in the corresponding position in the second strand. Example UNA oligomers of this disclosure are shown with 2-monomer length overhangs, although overhangs of from 1 to 8 monomers, or longer, can be used.

The symbol “X” in a strand or oligomer represents a UNA monomer. When a UNA monomer appears in one strand of the oligomer, and is unpaired with the other strand, the monomer can have any base attached. When a UNA monomer appears in one strand of the oligomer and is paired with a monomer in the other strand, the UNA monomer can have any base attached that would be complementary to the monomer in the corresponding paired position in the other strand.

Further, when the oligomer terminates in a UNA monomer, the terminal position has a 1-end, according to the UNA positional numbering shown above, instead of a 5′-end as for a nucleotide, or the terminal position has a 3-end, according to the positional numbering shown above, instead of a 3′-end as for a nucleotide.

For example, a UNA oligomer may have a UNA monomer at the 1-end on the first strand, a UNA monomer at the second position from the 3′ end of the first strand, and a UNA monomer at the second position from the 3′ end on the second strand, as follows:

(sense)
SEQ ID NO: 1
1-XNNNNNNNNNNNNNNNNNNXN-3′
(antisense)
SEQ ID NO: 2
3′-NXNNNNNNNNNNNNNNNNNNN-5′

Complementarity of strands can involve mismatches. In certain embodiments, complementarity of strands can include one to three, or more, mismatches.

In some embodiments, a UNA oligomer of this disclosure can have one or more UNA monomers at the 1-end of the first strand, and one or more UNA monomers at the 3-end of the first strand.

In further embodiments, a UNA oligomer of this disclosure can have one or more UNA monomers at the 3-end of the second strand.

In certain embodiments, a duplex UNA oligomer of this disclosure can have one or more UNA monomers at the 1-end of the first strand, one or more UNA monomers at the 3-end of the first strand, and one or more UNA monomers at the 3-end of the second strand.

A UNA oligomer of this disclosure the oligomer may have a first strand and a second strand, each of the strands independently being 19-23 monomers in length.

In certain embodiments, a UNA oligomer of this disclosure may have a first strand that is 19-23 monomers in length.

In certain embodiments, a UNA oligomer of this disclosure may have a duplex region that is 19-21 monomers in length.

In further embodiments, a UNA oligomer of this disclosure may have a second strand that is 19-23 monomers in length.

In certain embodiments, a UNA oligomer of this disclosure may have a first strand that is 19 monomers in length, and a second strand that is 21 monomers in length.

In certain embodiments, a UNA oligomer of this disclosure may have a first strand that is 20 monomers in length, and a second strand that is 21 monomers in length.

In certain embodiments, a UNA oligomer of this disclosure may have a first strand that is 21 monomers in length, and a second strand that is 21 monomers in length.

In certain embodiments, a UNA oligomer of this disclosure may have a first strand that is 22 monomers in length, and a second strand that is 21 monomers in length.

A UNA oligomer of this disclosure for inhibiting gene expression can have a first strand and a second strand, each of the strands being 19-29 monomers in length. The monomers can be UNA monomers and nucleic acid nucleoside monomers. The oligomer can have a duplex structure of from 14 to 29 monomers in length. The UNA oligomer can be targeted to a target gene and can exhibit reduced off-target effects as compared to a conventional siRNA. In some embodiments, a UNA oligomer of this disclosure can have a first strand and a second strand, each of the strands being 19-23 monomers in length.

In another aspect, the UNA oligomer may have a blunt end, or may have one or more overhangs. In some embodiments, the first and second strands may be connected with a connecting oligomer in between the strands and form a duplex region with a connecting loop at one end.

In certain embodiments, an overhang can be one or two monomers in length.

Examples of an overhang can contain one or more UNA monomers, natural nucleotides, non-natural nucleotides, modified nucleotides, or chemically-modified nucleotides, and combinations thereof.

Examples of an overhang can contain one or more deoxythymidine nucleotides.

Examples of an overhang can contain one or more 2′-O-methyl nucleotides, inverted abasic monomers, inverted thymidine monomers, L-thymidine monomers, or glyceryl nucleotides.

A UNA oligomer can mediate cleavage of a target nucleic acid in a cell. In some processes, the second strand of the UNA oligomer, at least a portion of which can be complementary to the target nucleic acid, can act as a guide strand that can hybridize to the target nucleic acid.

The second strand can be incorporated into an RNA Induced Silencing Complex (RISC).

A UNA oligomer of this disclosure may comprise naturally-occurring nucleic acid nucleotides, and modifications thereof that are compatible with gene silencing activity.

In some aspects, a UNA oligomer is a double stranded construct molecule that is able to inhibit gene expression.

As used herein, the term strand refers to a single, contiguous chain of monomers, the chain having any number of internal monomers and two end monomers, where each end monomer is attached to one internal monomer on one side and is not attached to a monomer on the other side, so that it ends the chain.

The monomers of a UNA oligomer may be attached via phosphodiester linkages, phosphorothioate linkages, gapped linkages, and other variations.

In some embodiments, a UNA oligomer can include mismatches in complementarity between the first and second strands. In other embodiments, a UNA oligomer may have 1, or 2, or 3 mismatches. The mismatches may occur at any position in the duplex region.

The target of a UNA oligomer can be a target nucleic acid of a target gene.

A UNA oligomer may have one or two overhangs outside the duplex region. The overhangs can be an unpaired portion at the end of the first strand or second strand. The lengths of the overhang portions of the first and second strands can be the same or different.

A UNA oligomer may have at least one blunt end. A blunt end does not have an overhang portion, and the duplex region at a blunt end terminates at the same position for both the first and second strands.

A UNA oligomer can be RISC length, which means that it has a duplex length of less than 25 base pairs.

In certain embodiments, a UNA oligomer can be a single strand that folds upon itself and hybridizes to itself to form a double stranded region having a connecting loop at the end of the double stranded region.

In some embodiments, an oligomeric compound of this disclosure may have a first strand and a second strand, each of the strands independently being 19-23 monomers in length, where any monomer that is not a UNA monomer can be a Q monomer.

In some embodiments, an oligomeric compound of this disclosure may have a first strand and a second strand, each of the strands independently being 19-23 monomers in length, where any monomer that is not a UNA monomer can be a Q monomer, and where the number of Q monomers is less than twenty.

In some embodiments, an oligomeric compound of this disclosure may have a first strand and a second strand, each of the strands independently being 19-23 monomers in length, where any monomer that is not a UNA monomer can be a Q monomer, and where the number of Q monomers is less than twelve.

In some embodiments, an oligomeric compound of this disclosure may have a first strand and a second strand, each of the strands independently being 19-23 monomers in length, where any monomer that is not a UNA monomer can be a Q monomer, and where the number of Q monomers is less than ten.

In some embodiments, an oligomeric compound of this disclosure may have a first strand and a second strand, each of the strands independently being 19-23 monomers in length, where any monomer that is not a UNA monomer can be a Q monomer, and where the number of Q monomers is less than eight.

In some embodiments, an oligomeric compound of this disclosure may have a first strand and a second strand, each of the strands independently being 19-23 monomers in length, where any monomer that is not a UNA monomer can be a Q monomer, and where the number of Q monomers is from 1 to 20.

In some embodiments, an oligomeric compound of this disclosure may have a first strand and a second strand, each of the strands independently being 19-23 monomers in length, where any monomer that is not a UNA monomer can be a Q monomer, and where the number of Q monomers is from 1 to 15.

In some embodiments, an oligomeric compound of this disclosure may have a first strand and a second strand, each of the strands independently being 19-23 monomers in length, where any monomer that is not a UNA monomer can be a Q monomer, and where the number of Q monomers is from 1 to 9.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is less than twenty.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is less than twelve.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is less than ten.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is less than eight.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is from 1 to 20.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is from 1 to 15.

In some embodiments, an oligomeric compound of this disclosure may have a first strand and a second strand, each of the strands independently being 19-23 monomers in length, where any monomer that is not a UNA monomer can be a 2′-O-Methyl modified ribonucleotide, and where the number of 2′-O-Methyl modified ribonucleotides is from 1 to 9.

Methods of this disclosure include the treatment and/or prevention of nonalcoholic steatohepatitis disease in a subject. A subject can be a mammalian subject, including a human subject.

### PDGFRB and PDGFRA Reference Polynucleotides

As used herein, “Ref Pos” refers to reference position, which is the numerical position of a reference polynucleotide of a PDGFRB genome. The reference position is the position in the reference polynucleotide that corresponds target-wise to the 5′ end (or 1 end for UNA) of the sense strand of the oligomeric compound or siRNA of this disclosure.

The reference positions are numerical nucleobase positions based on a reference genome.

Reference polynucleotides for PDGFRB used herein are as follows:

Homo sapiens PDGFRB, transcript variant 1, mRNA, NCBI Reference Sequence: NM_002609.3.

Mus musculus, beta polypeptide (Pdgfrb), transcript variant 1, mRNA, NCBI Reference Sequence: NM_001146268.1.

Macaca fascicularis PDGFRB, mRNA, NCBI Reference Sequence: XM 005558242.2. (NCBI predicted version) (cynomolgus monkey).

Rattus norvegicus, Pdgfb, mRNA, NCBI Reference Sequence: NM_031525.1.

Reference polynucleotides for PDGFRA used herein are as follows:

Homo sapiens PDGFRA, transcript variant 1, mRNA, NCBI Reference Sequence: NM_006206.5.

Mus musculus, alpha polypeptide (Pdgfra), transcript variant 1, mRNA, NCBI Reference Sequence: NM_001083316.2.

### UNA Oligomers Targeting PDGFRB

Examples of base sequences of this disclosure targeted to a PDGFRB genome are shown in Table 1.

In some embodiments, an oligomeric compound of this disclosure can be formed having a first strand and a second strand, each strand being 21 monomers in length. The first strand can have 19 contiguous monomers with a sequence of attached bases shown in Table 1 (sense), and two or more additional overhang monomers on the 3′ end (3 end for UNA). The second strand can have 19 contiguous monomers with a sequence of attached bases shown in Table 1 (antisense, same Ref Pos as first strand), and two or more additional overhang monomers on the 3′ end (3 end for UNA).

Overhang monomers can be any of NN, QQ, XX, NX, NQ, XN, XQ, QN, and QX. For example, XQ can be UNA-U/mU, or UNA-U/*/dT.

An oligomeric compound of this disclosure can be composed of monomers. The monomers can have attached bases. An oligomeric compound of this disclosure can have a sequence of attached bases. The sequences of bases shown in Table 1 do not indicate to which monomer each of the bases in the sequence is attached. Thus, each sequence shown in Table 1 refers to a large number of small molecules, each of which is composed of a number of UNA monomers, as well as nucleic acid monomers. The nucleic acid monomers can be chemically modified, including modifications in the bases appearing in Table 1.

In some aspects, an oligomeric compound of this disclosure can be described by a sequence of attached bases, for example as shown in Table 1, and substituted forms thereof. As used herein, substituted forms include differently substituted UNA monomers, as well as chemically modified nucleic acid monomers, as are further described herein.

In some embodiments, one or more of three monomers at each end of each strand can be connected by a phosphorothioate, a chiral phosphorothioate, or a phosphorodithioate linkage.

For example, a compound may have one phosphorothioate linkage between two monomers at the 5′ end of the first strand, one phosphorothioate linkage between two monomers at the 3′ end of the first strand, one phosphorothioate linkage between monomers at the second and third positions from the 3′ end of the first strand, and one phosphorothioate linkage between two monomers at the 3′ end of the second strand.

In certain embodiments, a compound may have two or three phosphorothioate linkages at the 5′ end of the first strand, two or three phosphorothioate linkages at the 3′ end of the first strand, and one phosphorothioate linkage at the 3′ end of the second strand.

In additional embodiments, a compound may have one to three phosphorothioate linkages at the 5′ end of the first strand, two or three phosphorothioate linkages at the 3′ end of the first strand, two phosphorothioate linkages at the 5′ end of the second strand, and two phosphorothioate linkages at the 3′ end of the second strand.

In some examples, a compound may have a deoxythymidine nucleotide at the 3′ end of the first strand, at the 3′ end of the second strand, or at both the 3′ end of the first strand and the 3′ end of the second strand.

In some aspects, a compound may contain one to five UNA monomers.

In certain aspects, a compound may contain three UNA monomers.

In some embodiments, a compound may contain a UNA monomer at the 1-end of the first strand (5′ end), a UNA monomer at the second position from the 3-end of the first strand (3′ end), and a UNA monomer at the second position from the 3 end (3′ end) of the second strand.

In additional embodiments, a compound may contain a UNA monomer at the 1-end of the first strand (5′ end), a UNA monomer at the 3-end of the first strand (3′ end), and a UNA monomer at the second position from the 3′ end of the second strand.

In certain embodiments, a compound may contain a UNA monomer at any one or more of positions 2 to 8 from the 5′ end of the second strand (seed region), in addition to one or more UNA monomers at any other positions.

In some aspects, a compound may contain one or more chemically modified nucleotides.

### PDGFRB-Targeted Base Sequences

Examples of base sequences of this disclosure targeted to a PDGFRB genome are shown in Table 1 (Based on NM_002609.3).

TABLE 1
PDGFRB-targeted base sequences

| | SEQ | | SEQ | |
|---|---|---|---|---|
| REF | ID | Sense (5′-3′) | ID | Antisense (5′-3′) |
| POS | NO: | SEQ ID NOS: 3 to 52 | NO: | SEQ ID NOS: 53 to 102 |
| 1094 | 3 | CUCCAGGUGUCAUCCAUCA | 53 | UGAUGGAUGACACCUGGAG |
| 1677 | 4 | CUGAGGUCCAGCUCUCCUU | 54 | AAGGAGAGCUGGACCUCAG |
| 1818 | 5 | CCUGCAGAGACCUCAAAAG | 55 | CUUUUGAGGUCUCUGCAGG |
| 2448 | 6 | CCCACCUGAACGUGGUCAA | 56 | UUGACCACGUUCAGGUGGG |
| 2984 | 7 | CUGGUCAAGAUCUGUGACU | 57 | UUGACCACGUUCAGGUGGG |
| 3092 | 8 | AUCUUCAACAGCCUCUACA | 58 | UGUAGAGGCUGUUGAAGAU |
| 3204 | 9 | AGUUCUACAAUGCCAUCAA | 59 | UUGAUGGCAUUGUAGAACU |
| 3258 | 10 | CCGACGAGAUCUAUGAGAU | 60 | AUCUCAUAGAUCUCGUCGG |
| 3167 | 11 | GGUGGCACCCCUUACCCAG | 61 | CUGGGUAAGGGGUGCCACC |
| 820 | 12 | CAUCUUUGUGCCAGAUCCC | 62 | GGGAUCUGGCACAAAGAUG |
| 1036 | 13 | CUGCAAAACCACCAUUGGG | 63 | CCCAAUGGUGGUUUUGCAG |
| 2198 | 14 | CAUGAGUACAUCUACGUGG | 64 | CCACGUAGAUGUACUCAUG |
| 2201 | 15 | GAGUACAUCUACGUGGACC | 65 | GGUCCACGUAGAUGUACUC |
| 3007 | 16 | CCUGGCUCGAGACAUCAUG | 66 | CAUGAUGUCUCGAGCCAGG |
| 3096 | 17 | UCAACAGCCUCUACACCAC | 67 | GUGGUGUAGAGGCUGUUGA |
| 1230 | 18 | GUGGGCGGCUGGUGGAGCC | 68 | GGCUCCACCAGCCGCCCAC |
| 1773 | 19 | GCUGUCGUGGCCGGGGCAU | 69 | AUGCCCCGGCCACGACAGC |
| 1937 | 20 | GAGGUGGUGAGCACACUGC | 70 | GCAGUGUGCUCACCACCUC |
| 2068 | 21 | GGUGAUCUCAGCCAUCCUG | 71 | CAGGAUGGCUGAGAUCACC |
| 2174 | 22 | AUUGAGUCUGUGAGCUCUG | 72 | CAGAGCUCACAGACUCAAU |
| 2473 | 23 | GGGGGCCUGCACCAAAGGA | 73 | UCCUUUGGUGCAGGCCCCC |
| 2534 | 24 | CUGGUGGACUACCUGCACC | 74 | GGUGCAGGUAGUCCACCAG |
| 2685 | 25 | GCUACAUGGACAUGAGCAA | 75 | UUGCUCAUGUCCAUGUAGC |
| 2728 | 26 | CAUGCUGGACAUGAAAGGA | 76 | UCCUUUCAUGUCCAGCAUG |
| 2784 | 27 | UGGCCCCCUACGAUAACUA | 77 | UAGUUAUCGUAGGGGGCCA |
| 2811 | 28 | CUGCCCCUGAGAGGACCUA | 78 | UAGGUCCUCUCAGGGGCAG |
| 2864 | 29 | UCGUGGGCUUCAGCUACCA | 79 | UGGUAGCUGAAGCCCACGA |
| 2921 | 30 | CCUCCAAGAACUGCGUCCA | 80 | UGGACGCAGUUCUUGGAGG |
| 2943 | 31 | GCCAGGAACGUGCUCAUCU | 81 | AGAUGAGCACGUUCCUGGC |
| 3027 | 32 | CUCAAACUACAUCUCCAAA | 82 | UUUGGAGAUGUAGUUUGAG |
| 3128 | 33 | CUACUCUGGGAGAUCUUCA | 83 | UGAAGAUCUCCCAGAGUAG |
| 3225 | 34 | GCAUGGCCCAGCCUGCCCA | 84 | UGGGCAGGCUGGGCCAUGC |
| 3286 | 35 | UGGGAAGAAAAGUUUGAGA | 85 | UCUCAAACUUUUCUUCCCA |
| 3320 | 36 | UCUCCCAGCUGGUGCUGCU | 86 | AGCAGCACCAGCUGGGAGA |
| 3353 | 37 | GCGAAGGCUACAAAAAGAA | 87 | UUCUUUUUGUAGCCUUCGC |
| 3388 | 38 | GUUUCUGAGGAGUGACCAC | 88 | GUGGUCACUCCUCAGAAAC |
| 3453 | 39 | CUCCGAUCCCCCCUGGACA | 89 | UGUCCAGGGGGGAUCGGAG |
| 3481 | 40 | AGCCCAAUGAGAGUGACAA | 90 | UUGUCACUCUCAUUGGGCU |
| 3532 | 41 | UACCUGACCCCAAACCCGA | 91 | UCGGGUUUGGGGUCAGGUA |
| 3602 | 42 | ACCCUGAAUGAAGUCAACA | 92 | UGUUGACUUCAUUCAGGGU |
| 3638 | 43 | GCCCCCUGGAGCCCCAAGA | 93 | UCUUGGGGCUCCAGGGGGC |
| 3763 | 44 | CAGAGGACAGCUUCCUGUA | 94 | UACAGGAAGCUGUCCUCUG |
| 3953 | 45 | CUUAGGAGGCAAGAAAACU | 95 | AGUUUUCUUGCCUCCUAAG |
| 4018 | 46 | GACUCUGAACCAGGGUUCC | 96 | GGAACCCUGGUUCAGAGUC |
| 4372 | 47 | CCAAGCUGGUCUGGGGCCA | 97 | UGGCCCCAGACCAGCUUGG |
| 4660 | 48 | UGCACUGGACCUGCUAUGA | 98 | UCAUAGCAGGUCCAGUGCA |
| 5013 | 49 | CCCCAAGGACACAGAAAGA | 99 | UCUUUCUGUGUCCUUGGGG |
| 5564 | 50 | UCACCUAGGUUUACAAAUA | 100 | UAUUUGUAAACCUAGGUGA |
| 5619 | 51 | UAUAUGCUGUUAAGUUUUU | 101 | AAAAACUUAACAGCAUAUA |
| 5690 | 52 | GAAAGAUUUUAAUAUUAAA | 102 | UUUAAUAUUAAAAUCUUUC |
| 5594 | 615 | CUCACGUUAACUCACAUUU | 616 | AAAUGUGAGUUAACGUGAG |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 2. Table 2 shows sequentially “sense” and “antisense” pairs, for example, SEQ ID NO:103 and 104 are a “sense” and “antisense” pair.

TABLE 2
UNA oligomers targeted to PDGFRB (Sense (S)-Antisense (AS))

| | SEQ | | |
|---|---|---|---|
| REF | ID | | PDGFRB (Sense (S)-Antisense (AS)) |
| POS | NO: | S / AS | (5′-3′) |
| 3092 | 103 | S | UNA-A/*/mUrCmUrUmCrAmArCrArGmCrCmUrCmUrAmCrA/*/iUNA-U/*/T |
| 3092 | 104 | AS | mUrGmUrAmGrAmGrGmCrUmGmUmUrGmArAmGrAmU/iUNA-U/*/T |
| 3092 | 105 | S | UNA-G/*/mArGmArGmCrAmUrCrUrUmCrAmArCmArGmCrC/*/iUNA-U/*/T |
| 3092 | 106 | AS | mGrGmCrUmGrUmUrGmArAmGmAmUrGmCrUmCrUmC/iUNA-U/*/T |
| 3092 | 107 | S | UNA-G/*/mArGmCrAmUrCmUrUrCrAmArCmArGmCrCmUrC/*/iUNA-U/*/T |
| 3092 | 108 | AS | mGrAmGrGmCrUmGrUmUrGmAmAmGrAmUrGmCrUmC/iUNA-U/*/T |
| 3092 | 109 | S | UNA-G/*/mCrAmUrCmUrUmCrArArCmArGmCrCmUrCmUrA/*/iUNA-U/*/T |
| 3092 | 110 | AS | mUrAmGrAmGrGmCrUmGrUmUmGmArAmGrAmUrGmC/iUNA-U/*/T |
| 3092 | 111 | S | UNA-C/*/mUrUmCrAmArCmArGrCrCmUrCmUrAmCrAmCrC/*/iUNA-U/*/T |
| 3092 | 112 | AS | mUrCmArUmArGmArUmCrUmCmGmUrCmGrGmArGmG/iUNA-U/*/T |
| 3092 | 113 | S | UNA-C/*/mUrUmCrAmArCmArGrCrCmUrCmUrAmCrAmCrC/*/iUNA-U/*/T |
| 3092 | 114 | AS | mGrUmGrGmUrGmUrAmGrAmGmGmCrUmGrUmUrGmA/iUNA-U/*/T |
| 3258 | 115 | S | UNA-C/*/mCrGmArCmGrAmGrArUrCmUrAmUrGmArGmArU/*/iUNA-U/*/T |
| 3258 | 116 | AS | mArUmCrUmCrAmUrAmGrAmUmCmUrCmGrUmCrGmG/iUNA-U/*/T |
| 3258 | 117 | S | UNA-U/*/mGrCmCrUmCrCmGrArCrGmArGmArUmCrUmArU/*/iUNA-U/*/T |
| 3258 | 118 | AS | mArUmArGmArUmCrUmCrGmUmCmGrGmArGmGrCmA/iUNA-U/*/T |
| 3258 | 119 | S | UNA-G/*/mCrCmUrCmCrGmArCrGrAmGrAmUrCmUrAmUrG/*/iUNA-U/*/T |
| 3258 | 120 | AS | mCrAmUrAmGrAmUrCmUrCmGmUmCrGmGrAmGrGmC/iUNA-U/*/T |
| 3258 | 121 | S | UNA-C/*/mCrUmCrCmGrAmCrGrArGmArUmCrUmArUmGrA/*/iUNA-U/*/T |
| 3258 | 122 | AS | mUrCmArUmArGmArUmCrUmCmGmUrCmGrGmArGmG/iUNA-U/*/T |
| 3258 | 123 | S | UNA-C/*/mUrCmCrGmArCmGrArGrAmUrCmUrAmUrGmArG/*/iUNA-U/*/T |
| 3258 | 124 | AS | mCrUmCrAmUrAmGrAmUrCmUmCmGrUmCrGmGrAmG/iUNA-U/*/T |
| 3258 | 125 | S | UNA-U/*/mCrCmGrAmCrGmArGrArUmCrUmArUmGrAmGrA/*/iUNA-U/*/T |
| 3258 | 126 | AS | mUrCmUrCmArUmArGmArUmCmUmCrGmUrCmGrGmA/iUNA-U/*/T |
| 3258 | 127 | S | UNA-C/*/mGrAmCrGmArGmArUrCrUmArUmGrAmGrAmUrC/*/iUNA-U/*/T |
| 3258 | 128 | AS | mGrAmUrCmUrCmArUmArGmAmUmCrUmCrGmUrCmG/iUNA-U/*/T |
| 3258 | 129 | S | UNA-G/*/mArCmGrAmGrAmUrCrUrAmUrGmArGmArUmCrA/*/iUNA-U/*/T |
| 3258 | 130 | AS | mUrGmArUmCrUmCrAmUrAmGmAmUrCmUrCmGrUmC/iUNA-U/*/T |
| 3258 | 131 | S | UNA-A/*/mCrGmArGmArUmCrUrArUmGrAmGrAmUrCmArU/*/iUNA-U/*/T |
| 3258 | 132 | AS | mArUmGrAmUrCmUrCmArUmAmGmArUmCrUmCrGmU/iUNA-U/*/T |
| 3258 | 133 | S | UNA-C/*/mGrAmGrAmUrCmUrArUrGmArGmArUmCrAmUrG/*/iUNA-U/*/T |
| 3258 | 134 | AS | mCrAmUrGmArUmCrUmCrAmUmAmGrAmUrCmUrCmG/iUNA-U/*/T |
| 3258 | 135 | S | UNA-G/*/mArGmArUmCrUmArUrGrAmGrAmUrCmArUmGrC/*/iUNA-U/*/T |
| 3258 | 136 | AS | mGrCmArUmGrAmUrCmUrCmAmUmArGmArUmCrUmC/iUNA-U/*/T |
| 3258 | 137 | S | UNA-A/*/mGrAmUrCmUrAmUrGrArGmArUmCrAmUrGmCrA/*/iUNA-U/*/T |
| 3258 | 138 | AS | mUrGmCrAmUrGmArUmCrUmCmAmUrAmGrAmUrCmU/iUNA-U/*/T |
| 3258 | 139 | S | UNA-G/*/mArUmCrUmArUmGrArGrAmUrCmArUmGrCmArG/*/iUNA-U/*/T |
| 3258 | 140 | AS | mCrUmGrCmArUmGrAmUrCmUmCmArUmArGmArUmC/iUNA-U/*/T |
| 3258 | 141 | S | UNA-A/*/mUrCmUrAmUrGmArGrArUmCrAmUrGmCrAmGrA/*/iUNA-U/*/T |
| 3258 | 142 | AS | mUrCmUrGmCrAmUrGmArUmCmUmCrAmUrAmGrAmU/iUNA-U/*/T |
| 3258 | 143 | S | UNA-U/*/mCrUmArUmGrAmGrArUrCmArUmGrCmArGmArA/*/iUNA-U/*/T |
| 3258 | 144 | AS | mUrUmCrUmGrCmArUmGrAmUmCmUrCmArUmArGmA/iUNA-U/*/T |
| 3258 | 145 | S | UNA-C/*/mUrAmUrGmArGmArUrCrAmUrGmCrAmGrAmArG/*/iUNA-U/*/T |
| 3258 | 146 | AS | mCrUmUrCmUrGmCrAmUrGmAmUmCrUmCrAmUrAmG/iUNA-U/*/T |

In Tables herein, rN refers to N, which is a ribonucleotide; mN refers to a chemically-modified 2′-OMe ribonucleotide; an asterisk * between characters refers to a phosphorothioate linkage; dN refers to a deoxyribonucleotide; f refers to a 2′-deoxy-2′-fluoro ribonucleotide, for example fU; T and dT refer to a 2′-deoxy T nucleotide. Designations that may be used herein include mA, mG, mC, and mU, which refer to the 2′-O-Methyl modified ribonucleotides.

The terms UNA-A, UNA-U, UNA-C, and UNA-G refer to UNA monomers. In some embodiments, a UNA monomer can be UNA-A (can be designated A), UNA-U (can be designated U), UNA-C (can be designated C̆) and UNA-G (can be designated Ã). The designation iUNA refers to internal UNA.

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 3. Table 3 shows “sense” sequences that are combined with an “antisense” sequence shown in Table 4. For example, SEQ ID NO:147 of Table 3 is combined with SEQ ID NO:180 of Table 4, SEQ ID NO:148 of Table 3 is combined with SEQ ID NO:181 of Table 4, etc.

TABLE 3
UNA oligomers targeted to PDGFRB (Sense (S))

| | SEQ | | |
|---|---|---|---|
| REF | ID | | PDGFRB (Sense (S)) |
| POS | NO: | S / AS | (5′-3′) |
| 2685 | 147 | S | UNA-G/*/mCrUmArCmArUmGrGrArCmArUmGrAmGrCmArA/*/UNA-U/*/dT |
| 2685 | 148 | S | UNA-C/*/mCrUmGrAmCrCmGrGrGrGmArGmArGmCrGmArC/*/UNA-U/*/dT |
| 2685 | 149 | S | UNA-C/*/mUrGmArCmCrGmGrGrGrAmGrAmGrCmGrAmCrG/*/UNA-U/*/dT |
| 2685 | 150 | S | UNA-U/*/mGrAmCrCmGrGmGrGrArGmArGmCrGmArCmGrG/*/UNA-U/*/dT |
| 2685 | 151 | S | UNA-G/*/mArCmCrGmGrGmGrArGrAmGrCmGrAmCrGmGrU/*/UNA-U/*/dT |
| 2685 | 152 | S | UNA-A/*/mCrCmGrGmGrGmArGrArGmCrGmArCmGrGmUrG/*/UNA-U/*/dT |
| 2685 | 153 | S | UNA-C/*/mCrGmGrGmGrAmGrArGrCmGrAmCrGmGrUmGrG/*/UNA-U/*/dT |
| 2685 | 154 | S | UNA-C/*/mGrGmGrGmArGmArGrCrGmArCmGrGmUrGmGrC/*/UNA-U/*/dT |
| 2685 | 155 | S | UNA-G/*/mGrGmGrAmGrAmGrCrGrAmCrGmGrUmGrGmCrU/*/UNA-U/*/dT |
| 2685 | 156 | S | UNA-G/*/mGrGmArGmArGmCrGrArCmGrGmUrGmGrCmUrA/*/UNA-U/*/dT |
| 2685 | 157 | S | UNA-G/*/mGrAmGrAmGrCmGrArCrGmGrUmGrGmCrUmArC/*/UNA-U/*/dT |
| 2685 | 158 | S | UNA-G/*/mArGmArGmCrGmArCrGrGmUrGmGrCmUrAmCrA/*/UNA-U/*/dT |
| 2685 | 159 | S | UNA-A/*/mGrAmGrCmGrAmCrGrGrUmGrGmCrUmArCmArU/*/UNA-U/*/dT |
| 2685 | 160 | S | UNA-G/*/mArGmCrGmArCmGrGrUrGmGrCmUrAmCrAmUrG/*/UNA-U/*/dT |
| 2685 | 161 | S | UNA-A/*/mGrCmGrAmCrGmGrUrGrGmCrUmArCmArUmGrG/*/UNA-U/*/dT |
| 2685 | 162 | S | UNA-G/*/mCrGmArCmGrGmUrGrGrCmUrAmCrAmUrGmGrA/*/UNA-U/*/dT |
| 2685 | 163 | S | UNA-C/*/mGrAmCrGmGrUmGrGrCrUmArCmArUmGrGmArC/*/UNA-U/*/dT |
| 2685 | 164 | S | UNA-G/*/mArCmGrGmUrGmGrCrUrAmCrAmUrGmGrAmCrA/*/UNA-U/*/dT |
| 2685 | 165 | S | UNA-A/*/mCrGmGrUmGrGmCrUrArCmArUmGrGmArCmArU/*/UNA-U/*/dT |
| 2685 | 166 | S | UNA-C/*/mGrGmUrGmGrCmUrArCrAmUrGmGrAmCrAmUrG/*/UNA-U/*/dT |
| 2685 | 167 | S | UNA-G/*/mGrUmGrGmCrUmArCrArUmGrGmArCmArUmGrA/*/UNA-U/*/dT |
| 2685 | 168 | S | UNA-G/*/mUrGmGrCmUrAmCrArUrGmGrAmCrAmUrGmArG/*/UNA-U/*/dT |
| 2685 | 169 | S | UNA-U/*/mGrGmCrUmArCmArUrGrGmArCmArUmGrAmGrC/*/UNA-U/*/dT |
| 2685 | 170 | S | UNA-G/*/mGrCmUrAmCrAmUrGrGrAmCrAmUrGmArGmCrA/*/UNA-U/*/dT |
| 2685 | 171 | S | UNA-C/*/mUrAmCrAmUrGmGrArCrAmUrGmArGmCrAmArG/*/UNA-U/*/dT |
| 2685 | 172 | S | UNA-U/*/mArCmArUmGrGmArCrArUmGrAmGrCmArAmGrG/*/UNA-U/*/dT |
| 2685 | 173 | S | UNA-A/*/mCrAmUrGmGrAmCrArUrGmArGmCrAmArGmGrA/*/UNA-U/*/dT |
| 2685 | 174 | S | UNA-C/*/mArUmGrGmArCmArUrGrAmGrCmArAmGrGmArC/*/UNA-U/*/dT |
| 2685 | 175 | S | UNA-A/*/mUrGmGrAmCrAmUrGrArGmCrAmArGmGrAmCrG/*/UNA-U/*/dT |
| 2685 | 176 | S | UNA-U/*/mGrGmArCmArUmGrArGrCmArAmGrGmArCmGrA/*/UNA-U/*/dT |
| 2685 | 177 | S | UNA-G/*/mGrAmCrAmUrGmArGrCrAmArGmGrAmCrGmArA/*/UNA-U/*/dT |
| 2685 | 178 | S | UNA-G/*/mArCmArUmGrAmGrCrArAmGrGmArCmGrAmArU/*/UNA-U/*/dT |
| 2685 | 179 | S | UNA-A/*/mCrAmUrGmArGmCrArArGmGrAmCrGmArAmUrC/*/UNA-U/*/dT |

TABLE 4
UNA oligomers targeted to PDGFRB (Antisense (AS))

| | SEQ | | |
|---|---|---|---|
| REF | ID | | PDGFRB (Antisense (AS)) |
| POS | NO: | S / AS | (5′-3′) |
| 2685 | 180 | AS | mUrUmGrCmUrCmArUmGrUmCmCmArUmGrUmArGmC/UNA-U/*/dT |
| 2685 | 181 | AS | mGrUmCrGmCrUmCrUmCrCmCmCmGrGmUrCmArGmG/UNA-U/*/dT |
| 2685 | 182 | AS | mCrGmUrCmGrCmUrCmUrCmCmCmCrGmGrUmCrAmG/UNA-U/*/dT |
| 2685 | 183 | AS | mCrCmGrUmCrGmCrUmCrUmCmCmCrCmGrGmUrCmA/UNA-U/*/dT |
| 2685 | 184 | AS | mArCmCrGmUrCmGrCmUrCmUmCmCrCmCrGmGrUmC/UNA-U/*/dT |
| 2685 | 185 | AS | mCrAmCrCmGrUmCrGmCrUmCmUmCrCmCrCmGrGmU/UNA-U/*/dT |
| 2685 | 186 | AS | mCrCmArCmCrGmUrCmGrCmUmCmUrCmCrCmCrGmG/UNA-U/*/dT |
| 2685 | 187 | AS | mGrCmCrAmCrCmGrUmCrGmCmUmCrUmCrCmCrCmG/UNA-U/*/dT |
| 2685 | 188 | AS | mArGmCrCmArCmCrGmUrCmGmCmUrCmUrCmCrCmC/UNA-U/*/dT |
| 2685 | 189 | AS | mUrAmGrCmCrAmCrCmGrUmCmGmCrUmCrUmCrCmC/UNA-U/*/dT |
| 2685 | 190 | AS | mGrUmArGmCrCmArCmCrGmUmCmGrCmUrCmrUrCmC/UNA-U/*/dT |
| 2685 | 191 | AS | mUrGmUrAmGrCmCrAmCrCmGmUmCrGmCrUmCrUmC/UNA-U/*/dT |
| 2685 | 192 | AS | mArUmGrUmArGmCrCmArCmCmGmUrCmGrCmUrCmU/UNA-U/*/dT |
| 2685 | 193 | AS | mCrAmUrGmUrAmGrCmCrAmCmCmGrUmCrGmCrUmC/UNA-U/*/dT |
| 2685 | 194 | AS | mCrCmArUmGrUmArGmCrCmAmCmCrGmUrCmGrCmU/UNA-U/*/dT |
| 2685 | 195 | AS | mUrCmCrAmUrGmUrAmGrCmCmAmCrCmGrUmCrGmC/UNA-U/*/dT |
| 2685 | 196 | AS | mGrUmCrCmArUmGrUmArGmCmCmArCmCrGmUrCmG/UNA-U/*/dT |
| 2685 | 197 | AS | mUrGmUrCmCrAmUrGmUrAmGmCmCrAmCrCmGrUmC/UNA-U/*/dT |
| 2685 | 198 | AS | mArUmGrUmCrCmArUmGrUmAmGmCrCmArCmCrGmU/UNA-U/*/dT |
| 2685 | 199 | AS | mCrAmUrGmUrCmCrAmUrGmUmAmGrCmCrAmCrCmG/UNA-U/*/dT |
| 2685 | 200 | AS | mUrCmArUmGrUmCrCmArUmGmUmArGmCrCmArCmC/UNA-U/*/dT |
| 2685 | 201 | AS | mCrUmCrAmUrGmUrCmCrAmUmGmUrAmGrCmCrAmC/UNA-U/*/dT |
| 2685 | 202 | AS | mGrCmUrCmArUmGrUmCrCmAmUmGrUmArGmCrCmA/UNA-U/*/dT |
| 2685 | 203 | AS | mUrGmCrUmCrAmUrGmUrCmCmAmUrGmUrAmGrCmC/UNA-U/*/dT |
| 2685 | 204 | AS | mCrUmUrGmCrUmCrAmUrGmUmCmCrAmUrGmUrAmG/UNA-U/*/dT |
| 2685 | 205 | AS | mCrCmUrUmGrCmUrCmArUmGmUmCrCmArUmGrUmA/UNA-U/*/dT |
| 2685 | 206 | AS | mUrCmCrUmUrGmCrUmCrAmUmGmUrCmCrAmUrGmU/UNA-U/*/dT |
| 2685 | 207 | AS | mGrUmCrCmUrUmGrCmUrCmAmUmGrUmCrCmArUmG/UNA-U/*/dT |
| 2685 | 208 | AS | mCrGmUrCmCrUmUrGmCrUmCmAmUrGmUrCmCrAmU/UNA-U/*/dT |
| 2685 | 209 | AS | mUrCmGrUmCrCmUrUmGrCmUmCmArUmGrUmCrCmA/UNA-U/*/dT |
| 2685 | 210 | AS | mUrUmCrGmUrCmCrUmUrGmCmUmCrAmUrGmUrCmC/UNA-U/*/dT |
| 2685 | 211 | AS | mArUmUrCmGrUmCrCmUrUmGmCmUrCmArUmGrUmC/UNA-U/*/dT |
| 2685 | 212 | AS | mGrAmUrUmCrGmUrCmCrUmUmGmCrUmCrAmUrGmU/UNA-U/*/dT |

### UNA Oligomers Targeting PDGFRB

Embodiments of this disclosure can provide oligomeric molecules that are active agents targeted to PDGFRB.

Examples of UNA oligomers of this disclosure that are targeted to PDGFRB are shown in Table 5. Table 5 shows “sense” sequences that are combined with an “antisense” sequence in Table 6. For example, SEQ ID NO:213 of Table 5 is combined with SEQ ID NO:256 of Table 6, SEQ ID NO:214 of Table 5 is combined with SEQ ID NO:257 of Table 6, etc.

TABLE 5
UNA oligomers targeted to PDGFRB (Sense (S))

| | SEQ | | |
|---|---|---|---|
| REF | ID | | PDGFRB (Sense (S)) |
| POS | NO: | S / AS | (5′-3′) |
| 3481 | 213 | S | UNA-A/*/mGrCmCrCmArAmUrGrArGmArGmUrGmArCmArA/*/UNA-U/*/dT |
| 3481 | 214 | S | UNA-C/*/mArGmCrUmCrCmGrUrCrCmUrCmUrAmCrAmCrC/*/UNA-U/*/dT |
| 3481 | 215 | S | UNA-A/*/mGrCmUrCmCrGmUrCrCrUmCrUmArCmArCmCrG/*/UNA-U/*/dT |
| 3481 | 216 | S | UNA-G/*/mCrUmCrCmGrUmCrCrUrCmUrAmCrAmCrCmGrC/*/UNA-U/*/dT |
| 3481 | 217 | S | UNA-C/*/mUrCmCrGmUrCmCrUrCrUmArCmArCmCrGmCrC/*/UNA-U/*/dT |
| 3481 | 218 | S | UNA-U/*/mCrCmGrUmCrCmUrCrUrAmCrAmCrCmGrCmCrG/*/UNA-U/*/dT |
| 3481 | 219 | S | UNA-C/*/mCrGmUrCmCrUmCrUrArCmArCmCrGmCrCmGrU/*/UNA-U/*/dT |
| 3481 | 220 | S | UNA-C/*/mGrUmCrCmUrCmUrArCrAmCrCmGrCmCrGmUrG/*/UNA-U/*/dT |
| 3481 | 221 | S | UNA-G/*/mUrCmCrUmCrUmArCrArCmCrGmCrCmGrUmGrC/*/UNA-U/*/dT |
| 3481 | 222 | S | UNA-U/*/mCrCmUrCmUrAmCrArCrCmGrCmCrGmUrGmCrA/*/UNA-U/*/dT |
| 3481 | 223 | S | UNA-C/*/mCrUmCrUmArCmArCrCrGmCrCmGrUmGrCmArG/*/UNA-U/*/dT |
| 3481 | 224 | S | UNA-C/*/mUrCmUrAmCrAmCrCrGrCmCrGmUrGmCrAmGrC/*/UNA-U/*/dT |
| 3481 | 225 | S | UNA-U/*/mCrUmArCmArCmCrGrCrCmGrUmGrCmArGmCrC/*/UNA-U/*/dT |
| 3481 | 226 | S | UNA-C/*/mUrAmCrAmCrCmGrCrCrGmUrGmCrAmGrCmCrC/*/UNA-U/*/dT |
| 3481 | 227 | S | UNA-U/*/mArCmArCmCrGmCrCrGrUmGrCmArGmCrCmCrA/*/UNA-U/*/dT |
| 3481 | 228 | S | UNA-A/*/mCrAmCrCmGrCmCrGrUrGmCrAmGrCmCrCmArA/*/UNA-U/*/dT |
| 3481 | 229 | S | UNA-C/*/mArCmCrGmCrCmGrUrGrCmArGmCrCmCrAmArU/*/UNA-U/*/dT |
| 3481 | 230 | S | UNA-A/*/mCrCmGrCmCrGmUrGrCrAmGrCmCrCmArAmUrG/*/UNA-U/*/dT |
| 3481 | 231 | S | UNA-C/*/mCrGmCrCmGrUmGrCrArGmCrCmCrAmArUmGrA/*/UNA-U/*/dT |
| 3481 | 232 | S | UNA-C/*/mGrCmCrGmUrGmCrArGrCmCrCmArAmUrGmArG/*/UNA-U/*/dT |
| 3481 | 233 | S | UNA-G/*/mCrCmGrUmGrCmArGrCrCmCrAmArUmGrAmGrA/*/UNA-U/*/dT |
| 3481 | 234 | S | UNA-C/*/mCrGmUrGmCrAmGrCrCrCmArAmUrGmArGmArG/*/UNA-U/*/dT |
| 3481 | 235 | S | UNA-C/*/mGrUmGrCmArGmCrCrCrAmArUmGrAmGrAmGrU/*/UNA-U/*/dT |
| 3481 | 236 | S | UNA-G/*/mUrGmCrAmGrCmCrCrArAmUrGmArGmArGmUrG/*/UNA-U/*/dT |
| 3481 | 237 | S | UNA-U/*/mGrCmArGmCrCmCrArArUmGrAmGrAmGrUmGrA/*/UNA-U/*/dT |
| 3481 | 238 | S | UNA-G/*/mCrAmGrCmCrCmArArUrGmArGmArGmUrGmArC/*/UNA-U/*/dT |
| 3481 | 239 | S | UNA-C/*/mArGmCrCmCrAmArUrGrAmGrAmGrUmGrAmCrA/*/UNA-U/*/dT |
| 3481 | 240 | S | UNA-G/*/mCrCmCrAmArUmGrArGrAmGrUmGrAmCrAmArC/*/UNA-U/*/dT |
| 3481 | 241 | S | UNA-C/*/mCrCmArAmUrGmArGrArGmUrGmArCmArAmCrG/*/UNA-U/*/dT |
| 3481 | 242 | S | UNA-C/*/mCrAmArUmGrAmGrArGrUmGrAmCrAmArCmGrA/*/UNA-U/*/dT |
| 3481 | 243 | S | UNA-C/*/mArAmUrGmArGmArGrUrGmArCmArAmCrGmArC/*/UNA-U/*/dT |
| 3481 | 244 | S | UNA-A/*/mArUmGrAmGrAmGrUrGrAmCrAmArCmGrAmCrU/*/UNA-U/*/dT |
| 3481 | 245 | S | UNA-A/*/mUrGmArGmArGmUrGrArCmArAmCrGmArCmUrA/*/UNA-U/*/dT |
| 3481 | 246 | S | UNA-U/*/mGrAmGrAmGrUmGrArCrAmArCmGrAmCrUmArC/*/UNA-U/*/dT |
| 3481 | 247 | S | UNA-G/*/mArGmArGmUrGmArCrArAmCrGmArCmUrAmCrA/*/UNA-U/*/dT |
| 3481 | 248 | S | UNA-A/*/mGrAmGrUmGrAmCrArArCmGrAmCrUmArCmArU/*/UNA-U/*/dT |
| 3481 | 249 | S | UNA-G/*/mArGmUrGmArCmArArCrGmArCmUrAmCrAmUrC/*/UNA-U/*/dT |
| 3481 | 250 | S | UNA-A/*/mGrUmGrAmCrAmArCrGrAmCrUmArCmArUmCrA/*/UNA-U/*/dT |
| 3481 | 251 | S | UNA-G/*/mUrGmArCmArAmCrGrArCmUrAmCrAmUrCmArU/*/UNA-U/*/dT |
| 3481 | 252 | S | UNA-U/*/mGrAmCrAmArCmGrArCrUmArCmArUmCrAmUrC/*/UNA-U/*/dT |
| 3481 | 253 | S | UNA-G/*/mArCmArAmCrGmArCrUrAmCrAmUrCmArUmCrC/*/UNA-U/*/dT |
| 3481 | 254 | S | UNA-A/*/mCrAmArCmGrAmCrUrArCmArUmCrAmUrCmCrC/*/UNA-U/*/dT |
| 3481 | 255 | S | UNA-C/*/mArAmCrGmArCmUrArCrAmUrCmArUmCrCmCrC/*/UNA-U/*/dT |

TABLE 6
UNA oligomers targeted to PDGFRB (Antisense (AS))

| | SEQ | | |
|---|---|---|---|
| REF | ID | | PDGFRB (Antisense (AS)) |
| POS | NO: | S / AS | (5′-3′) |
| 3481 | 256 | AS | mUrUmGrUmCrAmCrUmCrUmCmAmUrUmGrGmGrCmU/UNA-U/*/dT |
| 3481 | 257 | AS | mGrGmUrGmUrAmGrAmGrGmAmCmGrGmArGmCrUmG/UNA-U/*/dT |
| 3481 | 258 | AS | mCrGmGrUmGrUmArGmArGmGmAmCrGmGrAmGrCmU/UNA-U/*/dT |
| 3481 | 259 | AS | mGrCmGrGmUrGmUrAmGrAmGmGmArCmGrGmArGmC/UNA-U/*/dT |
| 3481 | 260 | AS | mGrGmCrGmGrUmGrUmArGmAmGmGrAmCrGmGrAmG/UNA-U/*/dT |
| 3481 | 261 | AS | mCrGmGrCmGrGmUrGmUrAmGmAmGrGmArCmGrGmA/UNA-U/*/dT |
| 3481 | 262 | AS | mArCmGrGmCrGmGrUmGrUmAmGmArGmGrAmCrGmG/UNA-U/*/dT |
| 3481 | 263 | AS | mCrAmCrGmGrCmGrGmUrGmUmAmGrAmGrGmArCmG/UNA-U/*/dT |
| 3481 | 264 | AS | mGrCmArCmGrGmCrGmGrUmGmUmArGmArGmGrAmC/UNA-U/*/dT |
| 3481 | 265 | AS | mUrGmCrAmCrGmGrCmGrGmUmGmUrAmGrAmGrGmA/UNA-U/*/dT |
| 3481 | 266 | AS | mCrUmGrCmArCmGrGmCrGmGmUmGrUmArGmArGmG/UNA-U/*/dT |
| 3481 | 267 | AS | mGrCmUrGmCrAmCrGmGrCmGmGmUrGmUrAmGrAmG/UNA-U/*/dT |
| 3481 | 268 | AS | mGrGmCrUmGrCmArCmGrGmCmGmGrUmGrUmArGmA/UNA-U/*/dT |
| 3481 | 269 | AS | mGrGmGrCmUrGmCrAmCrGmGmCmGrGmUrGmUrAmG/UNA-U/*/dT |
| 3481 | 270 | AS | mUrGmGrGmCrUmGrCmArCmGmGmCrGmGrUmGrUmA/UNA-U/*/dT |
| 3481 | 271 | AS | mUrUmGrGmGrCmUrGmCrAmCmGmGrCmGrGmUrGmU/UNA-U/*/dT |
| 3481 | 272 | AS | mArUmUrGmGrGmCrUmGrCmAmCmGrGmCrGmGrUmG/UNA-U/*/dT |
| 3481 | 273 | AS | mCrAmUrUmGrGmGrCmUrGmCmAmCrGmGrCmGrGmU/UNA-U/*/dT |
| 3481 | 274 | AS | mUrCmArUmUrGmGrGmCrUmGmCmArCmGrGmCrGmG/UNA-U/*/dT |
| 3481 | 275 | AS | mCrUmCrAmUrUmGrGmGrCmUmGmCrAmCrGmGrCmG/UNA-U/*/dT |
| 3481 | 276 | AS | mUrCmUrCmArUmUrGmGrGmCmUmGrCmArCmGrGmC/UNA-U/*/dT |
| 3481 | 277 | AS | mCrUmCrUmCrAmUrUmGrGmGmCmUrGmCrAmCrGmG/UNA-U/*/dT |
| 3481 | 278 | AS | mArCmUrCmUrCmArUmUrGmGmGmCrUmGrCmArCmG/UNA-U/*/dT |
| 3481 | 279 | AS | mCrAmCrUmCrUmCrAmUrUmGmGmGrCmUrGmCrAmC/UNA-U/*/dT |
| 3481 | 280 | AS | mUrCmArCmUrCmUrCmArUmUmGmGrGmCrUmGrCmA/UNA-U/*/dT |
| 3481 | 281 | AS | mGrUmCrAmCrUmCrUmCrAmUmUmGrGmGrCmUrGmC/UNA-U/*/dT |
| 3481 | 282 | AS | mUrGmUrCmArCmUrCmUrCmAmUmUrGmGrGmCrUmG/UNA-U/*/dT |
| 3481 | 283 | AS | mGrUmUrGmUrCmArCmUrCmUmCmArUmUrGmGrGmC/UNA-U/*/dT |
| 3481 | 284 | AS | mCrGmUrUmGrUmCrAmCrUmCmUmCrAmUrUmGrGmG/UNA-U/*/dT |
| 3481 | 285 | AS | mUrCmGrUmUrGmUrCmArCmUmCmUrCmArUmUrGmG/UNA-U/*/dT |
| 3481 | 286 | AS | mGrUmCrGmUrUmGrUmCrAmCmUmCrUmCrAmUrUmG/UNA-U/*/dT |
| 3481 | 287 | AS | mArGmUrCmGrUmUrGmUrCmAmCmUrCmUrCmArUmU/UNA-U/*/dT |
| 3481 | 288 | AS | mUrAmGrUmCrGmUrUmGrUmCmAmCrUmCrUmCrAmU/UNA-U/*/dT |
| 3481 | 289 | AS | mGrUmArGmUrCmGrUmUrGmUmCmArCmUrCmUrCmA/UNA-U/*/dT |
| 3481 | 290 | AS | mUrGmUrAmGrUmCrGmUrUmGmUmCrAmCrUmCrUmC/UNA-U/*/dT |
| 3481 | 291 | AS | mArUmGrUmArGmUrCmGrUmUmGmUrCmArCmUrCmU/UNA-U/*/dT |
| 3481 | 292 | AS | mGrAmUrGmUrAmGrUmCrGmUmUmGrUmCrAmCrUmC/UNA-U/*/dT |
| 3481 | 293 | AS | mUrGmArUmGrUmArGmUrCmGmUmUrGmUrCmArCmU/UNA-U/*/dT |
| 3481 | 294 | AS | mArUmGrAmUrGmUrAmGrUmCmGmUrUmGrUmCrAmC/UNA-U/*/dT |
| 3481 | 295 | AS | mGrAmUrGmArUmGrUmArGmUmCmGrUmUrGmUrCmA/UNA-U/*/dT |
| 3481 | 296 | AS | mGrGmArUmGrAmUrGmUrAmGmUmCrGmUrUmGrUmC/UNA-U/*/dT |
| 3481 | 297 | AS | mGrGmGrAmUrGmArUmGrUmAmGmUrCmGrUmUrGmU/UNA-U/*/dT |
| 3481 | 298 | AS | mGrGmGrGmArUmGrAmUrGmUmAmGrUmCrGmUrUmG/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 7. Table 7 shows “sense” sequences that are combined with an “antisense” sequence in Table 8. For example, SEQ ID NO:299 of Table 7 is combined with SEQ ID NO:317 of Table 8, SEQ ID NO:300 of Table 7 is combined with SEQ ID NO:318 of Table 8, etc.

TABLE 7
UNA oligomers targeted to PDGFRB (Sense (S))

| | SEQ | | |
|---|---|---|---|
| REF | ID | | PDGFRB (Sense (S)) |
| POS | NO: | S / AS | (5′-3′) |
| 3602 | 299 | S | UNA-A/*/mCrCmCrUmGrAmArUrGrAmArGmUrCmArAmCrA/*/UNA-U/*/dT |
| 3602 | 300 | S | UNA-A/*/mGrCmCrAmGrCmUrCrCrAmCrCmCrUmGrAmArU/*/UNA-U/*/dT |
| 3602 | 301 | S | UNA-G/*/mCrCmArGmCrUmCrCrArCmCrCmUrGmArAmUrG/*/UNA-U/*/dT |
| 3602 | 302 | S | UNA-C/*/mCrAmGrCmUrCmCrArCrCmCrUmGrAmArUmGrA/*/UNA-U/*/dT |
| 3602 | 303 | S | UNA-C/*/mArGmCrUmCrCmArCrCrCmUrGmArAmUrGmArA/*/UNA-U/*/dT |
| 3602 | 304 | S | UNA-A/*/mGrCmUrCmCrAmCrCrCrUmGrAmArUmGrAmArG/*/UNA-U/*/dT |
| 3602 | 305 | S | UNA-G/*/mCrUmCrCmArCmCrCrUrGmArAmUrGmArAmGrU/*/UNA-U/*/dT |
| 3602 | 306 | S | UNA-C/*/mUrCmCrAmCrCmCrUrGrAmArUmGrAmArGmUrC/*/UNA-U/*/dT |
| 3602 | 307 | S | UNA-U/*/mCrCmArCmCrCmUrGrArAmUrGmArAmGrUmCrA/*/UNA-U/*/dT |
| 3602 | 308 | S | UNA-C/*/mCrAmCrCmCrUmGrArArUmGrAmArGmUrCmArA/*/UNA-U/*/dT |
| 3602 | 309 | S | UNA-C/*/mArCmCrCmUrGmArArUrGmArAmGrUmCrAmArC/*/UNA-U/*/dT |
| 3602 | 310 | S | UNA-C/*/mCrCmUrGmArAmUrGrArAmGrUmCrAmArCmArC/*/UNA-U/*/dT |
| 3602 | 311 | S | UNA-C/*/mCrUmGrAmArUmGrArArGmUrCmArAmCrAmCrC/*/UNA-U/*/dT |
| 3602 | 312 | S | UNA-C/*/mUrGmArAmUrGmArArGrUmCrAmArCmArCmCrU/*/UNA-U/*/dT |
| 3602 | 313 | S | UNA-U/*/mGrAmArUmGrAmArGrUrCmArAmCrAmCrCmUrC/*/UNA-U/*/dT |
| 3602 | 314 | S | UNA-G/*/mArAmUrGmArAmGrUrCrAmArCmArCmCrUmCrC/*/UNA-U/*/dT |
| 3602 | 315 | S | UNA-A/*/mArUmGrAmArGmUrCrArAmCrAmCrCmUrCmCrU/*/UNA-U/*/dT |
| 3602 | 316 | S | UNA-A/*/mUrGmArAmGrUmCrArArCmArCmCrUmCrCmUrC/*/UNA-U/*/dT |

TABLE 8
UNA oligomers targeted to PDGFRB (Antisense (AS))

| | SEQ | | |
|---|---|---|---|
| REF | ID | | PDGFRB (Antisense (AS)) |
| POS | NO: | S / AS | (5′-3′) |
| 3602 | 317 | AS | mUrGmUrUmGrAmCrUmUrCmAmUmUrCmArGmGrGmU/UNA-U/*/dT |
| 3602 | 318 | AS | mArUmUrCmArGmGrGmUrGmGmAmGrCmUrGmGrCmU/UNA-U/*/dT |
| 3602 | 319 | AS | mCrAmUrUmCrAmGrGmGrUmGmGmArGmCrUmGrGmC/UNA-U/*/dT |
| 3602 | 320 | AS | mUrCmArUmUrCmArGmGrGmUmGmGrAmGrCmUrGmG/UNA-U/*/dT |
| 3602 | 321 | AS | mUrUmCrAmUrUmCrAmGrGmGmUmGrGmArGmCrUmG/UNA-U/*/dT |
| 3602 | 322 | AS | mCrUmUrCmArUmUrCmArGmGmGmUrGmGrAmGrCmU/UNA-U/*/dT |
| 3602 | 323 | AS | mArCmUrUmCrAmUrUmCrAmGmGmGrUmGrGmArGmC/UNA-U/*/dT |
| 3602 | 324 | AS | mGrAmCrUmUrCmArUmUrCmAmGmGrGmUrGmGrAmG/UNA-U/*/dT |
| 3602 | 325 | AS | mUrGmArCmUrUmCrAmUrUmCmAmGrGmGrUmGrGmA/UNA-U/*/dT |
| 3602 | 326 | AS | mUrUmGrAmCrUmUrCmArUmUmCmArGmGrGmUrGmG/UNA-U/*/dT |
| 3602 | 327 | AS | mGrUmUrGmArCmUrUmCrAmUmUmCrAmGrGmGrUmG/UNA-U/*/dT |
| 3602 | 328 | AS | mGrUmGrUmUrGmArCmUrUmCmAmUrUmCrAmGrGmG/UNA-U/*/dT |
| 3602 | 329 | AS | mGrGmUrGmUrUmGrAmCrUmUmCmArUmUrCmArGmG/UNA-U/*/dT |
| 3602 | 330 | AS | mArGmGrUmGrUmUrGmArCmUmUmCrAmUrUmCrAmG/UNA-U/*/dT |
| 3602 | 331 | AS | mGrAmGrGmUrGmUrUmGrAmCmUmUrCmArUmUrCmA/UNA-U/*/dT |
| 3602 | 332 | AS | mGrGmArGmGrUmGrUmUrGmAmCmUrUmCrAmUrUmC/UNA-U/*/dT |
| 3602 | 333 | AS | mArGmGrAmGrGmUrGmUrUmGmAmCrUmUrCmArUmU/UNA-U/*/dT |
| 3602 | 334 | AS | mGrAmGrGmArGmGrUmGrUmUmGmArCmUrUmCrAmU/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 9. Table 9 shows “sense” sequences that are combined with an “antisense” sequence in Table 10. For example, SEQ ID NO:335 of Table 9 is combined with SEQ ID NO:341 of Table 10, SEQ ID NO:336 of Table 9 is combined with SEQ ID NO:342 of Table 10, etc.

TABLE 9
UNA oligomers targeted to PDGFRB (Sense (S))

| | SEQ | | |
|---|---|---|---|
| REF | ID | | PDGFRB (Sense (S)) |
| POS | NO: | S / AS | (5′-3′) |
| 5564 | 335 | S | UNA-U/*/mCrAmCrCmUrAmGrGrUrUmUrAmCrAmArAmUrA/*/UNA-U/*/dT |
| 5564 | 336 | S | UNA-A/*/mArUmCrAmCrCmUrArGrGmUrUmUrAmCrAmArA/*/UNA-U/*/dT |
| 5564 | 337 | S | UNA-A/*/mUrCmArCmCrUmArGrGrUmUrUmArCmArAmArU/*/UNA-U/*/dT |
| 5564 | 338 | S | UNA-C/*/mArCmCrUmArGmGrUrUrUmArCmArAmArUmArC/*/UNA-U/*/dT |
| 5564 | 339 | S | UNA-A/*/mCrCmUrAmGrGmUrUrUrAmCrAmArAmUrAmCrU/*/UNA-U/*/dT |
| 5564 | 340 | S | UNA-C/*/mCrUmArGmGrUmUrUrArCmArAmArUmArCmUrU/*/UNA-U/*/dT |

TABLE 10
UNA oligomers targeted to PDGFRB (Antisense (AS))

| REF | SEQ | | |
|---|---|---|---|
| POS | ID NO: | S/AS | PDGFRB (Sense (AS) (5′-3′) |
| 5564 | 341 | AS | mUrAmUrUmUrGmUrAmArAmCmCmUrAmGrGmUrGmA/UNA-U/*/dT |
| 5564 | 342 | AS | mUrUmUrGmUrAmArAmCrCmUmAmGrGmUrGmArUmU/UNA-U/*/dT |
| 5564 | 343 | AS | mArUmUrUmGrUmArAmArCmCmUmArGmGrUmGrAmU/UNA-U/*/dT |
| 5564 | 344 | AS | mGrUmArUmUrUmGrUmArAmAmCmCrUmArGmGrUmG/UNA-U/*/dT |
| 5564 | 345 | AS | mArGmUrAmUrUmUrGmUrAmAmAmCrCmUrAmGrGmU/UNA-U/*/dT |
| 5564 | 346 | AS | mArAmGrUmArUmUrUmGrUmAmAmArCmCrUmArGmG/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 11. Table 11 shows “sense” sequences that are combined with an “antisense” sequence in Table 12. For example, SEQ ID NO:347 of Table 11 is combined with SEQ ID NO:380 of Table 12, SEQ ID NO:348 of Table 11 is combined with SEQ ID NO:381 of Table 12, etc.

TABLE 11
UNA oligomers targeted to PDGFRB (Sense (S))

| REF | SEQ | | |
|---|---|---|---|
| POS | ID NO: | S/AS | PDGFRB (Sense (S) (5′-3′) |
| 2685 | 347 | S | UNA-G/*/mCfUmAfCmAfUmGfGfAfCmAfUmGfAmGfCmAfA/*/UNA-U/*/dT |
| 2685 | 348 | S | UNA-C/*/mCfUmGfAmCfCmGfGfGfGmAfGmAfGmCfGmAfC/*/UNA-U/*/dT |
| 2685 | 349 | S | UNA-C/*/mUfGmAfCmCfGmGfGfGfAmGfAmGfCmGfAmCfG/*/UNA-U/*/dT |
| 2685 | 350 | S | UNA-U/*/mGfAmCfCmGfGmGfGfAfGmAfGmCfGmAfCmGfG/*/UNA-U/*/dT |
| 2685 | 351 | S | UNA-G/*/mAfCmCfGmGfGmGfAfGfAmGfCmGfAmCfGmGfU/*/UNA-U/*/dT |
| 2685 | 352 | S | UNA-A/*/mCfCmGfGmGfGmAfGfAfGmCfGmAfCmGfGmUfG/*/UNA-U/*/dT |
| 2685 | 353 | S | UNA-C/*/mCfGmGfGmGfAmGfAfGfCmGfAmCfGmGfUmGfG/*/UNA-U/*/dT |
| 2685 | 354 | S | UNA-C/*/mGfGmGfGmAfGmAfGfCfGmAfCmGfGmUfGmGfC/*/UNA-U/*/dT |
| 2685 | 355 | S | UNA-G/*/mGfGmGfAmGfAmGfCfGfAmCfGmGfUmGfGmCfU/*/UNA-U/*/dT |
| 2685 | 356 | S | UNA-G/*/mGfGmAfGmAfGmCfGfAfCmGfGmUfGmGfCmUfA/*/UNA-U/*/dT |
| 2685 | 357 | S | UNA-G/*/mGfAmGfAmGfCmGfAfCfGmGfUmGfGmCfUmAfC/*/UNA-U/*/dT |
| 2685 | 358 | S | UNA-G/*/mAfGmAfGmCfGmAfCfGfGmUfGmGfCmUfAmCfA/*/UNA-U/*/dT |
| 2685 | 359 | S | UNA-A/*/mGfAmGfCmGfAmCfGfGfUmGfGmCfUmAfCmAfU/*/UNA-U/*/dT |
| 2685 | 360 | S | UNA-G/*/mAfGmCfGmAfCmGfGfUfGmGfCmUfAmCfAmUfG/*/UNA-U/*/dT |
| 2685 | 361 | S | UNA-A/*/mGfCmGfAmCfGmGfUfGfGmCfUmAfCmAfUmGfG/*/UNA-U/*/dT |
| 2685 | 362 | S | UNA-G/*/mCfGmAfCmGfGmUfGfGfCmUfAmCfAmUfGmGfA/*/UNA-U/*/dT |
| 2685 | 363 | S | UNA-C/*/mGfAmCfGmGfUmGfGfCfUmAfCmAfUmGfGmAfC/*/UNA-U/*/dT |
| 2685 | 364 | S | UNA-G/*/mAfCmGfGmUfGmGfCfUfAmCfAmUfGmGfAmCfA/*/UNA-U/*/dT |
| 2685 | 365 | S | UNA-A/*/mCfGmGfUmGfGmCfUfAfCmAfUmGfGmAfCmAfU/*/UNA-U/*/dT |
| 2685 | 366 | 5 | UNA-C/*/mGfGmUfGmGfCmUfAfCfAmUfGmGfAmCfAmUfG/*/UNA-U/*/dT |
| 2685 | 367 | S | UNA-G/*/mGfUmGfGmCfUmAfCfAfUmGfGmAfCmAfUmGfA/*/UNA-U/*/dT |
| 2685 | 368 | S | UNA-G/*/mUfGmGfCmUfAmCfAfUfGmGfAmCfAmUfGmAfG/*/UNA-U/*/dT |
| 2685 | 369 | S | UNA-U/*/mGfGmCfUmAfCmAfUfGfGmAfCmAfUmGfAmGfC/*/UNA-U/*/dT |
| 2685 | 370 | S | UNA-G/*/mGfCmUfAmCfAmUfGfGfAmCfAmUfGmAfGmCfA/*/UNA-U/*/dT |
| 2685 | 371 | S | UNA-C/*/mUfAmCfAmUfGmGfAfCfAmUfGmAfGmCfAmAfG/*/UNA-U/*/dT |
| 2685 | 372 | S | UNA-U/*/mAfCmAfUmGfGmAfCfAfUmGfAmGfCmAfAmGfG/*/UNA-U/*/dT |
| 2685 | 373 | S | UNA-A/*/mCfAmUfGmGfAmCfAfUfGmAfGmCfAmAfGmGfA/*/UNA-U/*/dT |
| 2685 | 374 | S | UNA-C/*/mAfUmGfGmAfCmAfUfGfAmGfCmAfAmGfGmAfC/*/UNA-U/*/dT |
| 2685 | 375 | S | UNA-A/*/mUfGmGfAmCfAmUfGfAfGmCfAmAfGmGfAmCfG/*/UNA-U/*/dT |
| 2685 | 376 | S | UNA-U/*/mGfGmAfCmAfUmGfAfGfCmAfAmGfGmAfCmGfA/*/UNA-U/*/dT |
| 2685 | 377 | S | UNA-G/*/mGfAmCfAmUfGmAfGfCfAmAfGmGfAmCfGmAfA/*/UNA-U/*/dT |
| 2685 | 378 | S | UNA-G/*/mAfCmAfUmGfAmGfCfAfAmGfGmAfCmGfAmAfU/*/UNA-U/*/dT |
| 2685 | 379 | S | UNA-A/*/mCfAmUfGmAfGmCfAfAfGmGfAmCfGmAfAmUfC/*/UNA-U/*/dT |

TABLE 12
UNA oligomers targeted to PDGFRB (Antisense (AS))

| REF | SEQ | | |
|---|---|---|---|
| POS | ID NO: | S/AS | PDGFRB (Antisense (AS)) (5′-3′) |
| 2685 | 380 | AS | mUfUmGfCmUfCmAfUmGfUmCmCmAfUmGfUmAfGmC/UNA-U/*/dT |
| 2685 | 381 | AS | mGfUmCfGmCfUmCfUmCfCmCmCmGfGmUfCmAfGmG/UNA-U/*/dT |
| 2685 | 382 | AS | mCfGmUfCmGfCmUfCmUfCmCmCmCfGmGfUmCfAmG/UNA-U/*/dT |
| 2685 | 383 | AS | mCfCmGfUmCfGmCfUmCfUmCmCmCfCmGfGmUfCmA/UNA-U/*/dT |
| 2685 | 384 | AS | mAfCmCfGmUfCmGfCmUfCmUmCmCfCmCfGmGfUmC/UNA-U/*/dT |
| 2685 | 385 | AS | mCfAmCfCmGfUmCfGmCfUmCmUmCfCmCfCmGfGmU/UNA-U/*/dT |
| 2685 | 386 | AS | mCfCmAfCmCfGmUfCmGfCmUmCmUfCmCfCmCfGmG/UNA-U/*/dT |
| 2685 | 387 | AS | mGfCmCfAmCfCmGfUmCfGmCmUmCfUmCfCmCfCmG/UNA-U/*/dT |
| 2685 | 388 | AS | mAfGmCfCmAfCmCfGmUfCmGmCmUfCmUfCmCfCmC/UNA-U/*/dT |
| 2685 | 389 | AS | mUfAmGfCmCfAmCfCmGfUmCmGmCfUmCfUmCfCmC/UNA-U/*/dT |
| 2685 | 390 | AS | mGfUmAfGmCfCmAfCmCfGmUmCmGfCmUfCmfUfCmC/UNA-U/*/dT |
| 2685 | 391 | AS | mUfGmUfAmGfCmCfAmCfCmGmUmCfGmCfUmCfUmC/UNA-U/*/dT |
| 2685 | 392 | AS | mAfUmGfUmAfGmCfCmAfCmCmGmUfCmGfCmUfCmU/UNA-U/*/dT |
| 2685 | 393 | AS | mCfAmUfGmUfAmGfCmCfAmCmCmGfUmCfGmCfUmC/UNA-U/*/dT |
| 2685 | 394 | AS | mCfCmAfUmGfUmAfGmCfCmAmCmCfGmUfCmGfCmU/UNA-U/*/dT |
| 2685 | 395 | AS | mUfCmCfAmUfGmUfAmGfCmCmAmCfCmGfUmCfGmC/UNA-U/*/dT |
| 2685 | 396 | AS | mGfUmCfCmAfUmGfUmAfGmCmCmAfCmCfGmUfCmG/UNA-U/*/dT |
| 2685 | 397 | AS | mUfGmUfCmCfAmUfGmUfAmGmCmCfAmCfCmGfUmC/UNA-U/*/dT |
| 2685 | 398 | AS | mAfUmGfUmCfCmAfUmGfUmAmGmCfCmAfCmCfGmU/UNA-U/*/dT |
| 2685 | 399 | AS | mCfAmUfGmUfCmCfAmUfGmUmAmGfCmCfAmCfCmG/UNA-U/*/dT |
| 2685 | 400 | AS | mUfCmAfUmGfUmCfCmAfUmGmUmAfGmCfCmAfCmC/UNA-U/*/dT |
| 2685 | 401 | AS | mCfUmCfAmUfGmUfCmCfAmUmGmUfAmGfCmCfAmC/UNA-U/*/dT |
| 2685 | 402 | AS | mGfCmUfCmAfUmGfUmCfCmAmUmGfUmAfGmCfCmA/UNA-U/*/dT |
| 2685 | 403 | AS | mUfGmCfUmCfAmUfGmUfCmCmAmUfGmUfAmGfCmC/UNA-U/*/dT |
| 2685 | 404 | AS | mCfUmUfGmCfUmCfAmUfGmUmCmCfAmUfGmUfAmG/UNA-U/*/dT |
| 2685 | 405 | AS | mCfCmUfUmGfCmUfCmAfUmGmUmCfCmAfUmGfUmA/UNA-U/*/dT |
| 2685 | 406 | AS | mUfCmCfUmUfGmCfUmCfAmUmGmUfCmCfAmUfGmU/UNA-U/*/dT |
| 2685 | 407 | AS | mGfUmCfCmUfUmGfCmUfCmAmUmGfUmCfCmAfUmG/UNA-U/*/dT |
| 2685 | 408 | AS | mCfGmUfCmCfUmUfGmCfUmCmAmUfGmUfCmCfAmU/UNA-U/*/dT |
| 2685 | 409 | AS | mUfCmGfUmCfCmUfUmGfCmUmCmAfUmGfUmCfCmA/UNA-U/*/dT |
| 2685 | 410 | AS | mUfUmCfGmUfCmCfUmUfGmCmUmCfAmUfGmUfCmC/UNA-U/*/dT |
| 2685 | 411 | AS | mAfUmUfCmGfUmCfCmUfUmGmCmUfCmAfUmGfUmC/UNA-U/*/dT |
| 2685 | 412 | AS | mGfAmUfUmCfGmUfCmCfUmUmGmCfUmCfAmUfGmU/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 13. Table 13 shows “sense” sequences that are combined with an “antisense” sequence in Table 14. For example, SEQ ID NO:413 of Table 13 is combined with SEQ ID NO:456 of Table 14, SEQ ID NO:414 of Table 13 is combined with SEQ ID NO:457 of Table 14, etc.

TABLE 13
UNA oligomers targeted to PDGFRB (Sense (S))

| REF | SEQ | | |
|---|---|---|---|
| POS | ID NO: | S/AS | PDGFRB (Sense (S)) (5′-3′) |
| 3481 | 413 | S | UNA-A/*/mGfCmCfCmAfAmUfGfAfGmAfGmUfGmAfCmAfA/*/UNA-U/*/dT |
| 3481 | 414 | S | UNA-C/*/mAfGmCfUmCfCmGfUfCfCmUfCmUfAmCfAmCfC/*/UNA-U/*/dT |
| 3481 | 415 | S | UNA-A/*/mGfCmUfCmCfGmUfCfCfUmCfUmAfCmAfCmCfG/*/UNA-U/*/dT |
| 3481 | 416 | S | UNA-G/*/mCfUmCfCmGfUmCfCfUfCmUfAmCfAmCfCmGfC/*/UNA-U/*/dT |
| 3481 | 417 | S | UNA-C/*/mUfCmCfGmUfCmCfUfCfUmAfCmAfCmCfGmCfC/*/UNA-U/*/dT |
| 3481 | 418 | S | UNA-U/*/mCfCmGfUmCfCmUfCfUfAmCfAmCfCmGfCmCfG/*/UNA-U/*/dT |
| 3481 | 419 | S | UNA-C/*/mCfGmUfCmCfUmCfUfAfCmAfCmCfGmCfCmGfU/*/UNA-U/*/dT |
| 3481 | 420 | S | UNA-C/*/mGfUmCfCmUfCmUfAfCfAmCfCmGfCmCfGmUfG/*/UNA-U/*/dT |
| 3481 | 421 | S | UNA-G/*/mUfCmCfUmCfUmAfCfAfCmCfGmCfCmGfUmGfC/*/UNA-U/*/dT |
| 3481 | 422 | S | UNA-U/*/mCfCmUfCmUfAmCfAfCfCmGfCmCfGmUfGmCfA/*/UNA-U/*/dT |
| 3481 | 423 | S | UNA-C/*/mCfUmCfUmAfCmAfCfCfGmCfCmGfUmGfCmAfG/*/UNA-U/*/dT |
| 3481 | 424 | S | UNA-C/*/mUfCmUfAmCfAmCfCfGfCmCfGmUfGmCfAmGfC/*/UNA-U/*/dT |
| 3481 | 425 | S | UNA-U/*/mCfUmAfCmAfCmCfGfCfCmGfUmGfCmAfGmCfC/*/UNA-U/*/dT |
| 3481 | 426 | S | UNA-C/*/mUfAmCfAmCfCmGfCfCfGmUfGmCfAmGfCmCfC/*/UNA-U/*/dT |
| 3481 | 427 | S | UNA-U/*/mAfCmAfCmCfGmCfCfGfUmGfCmAfGmCfCmCfA/*/UNA-U/*/dT |
| 3481 | 428 | S | UNA-A/*/mCfAmCfCmGfCmCfGfUfGmCfAmGfCmCfCmAfA/*/UNA-U/*/dT |
| 3481 | 429 | S | UNA-C/*/mAfCmCfGmCfCmGfUfGfCmAfGmCfCmCfAmAfU/*/UNA-U/*/dT |
| 3481 | 430 | S | UNA-A/*/mCfCmGfCmCfGmUfGfCfAmGfCmCfCmAfAmUfG/*/UNA-U/*/dT |
| 3481 | 431 | S | UNA-C/*/mCfGmCfCmGfUmGfCfAfGmCfCmCfAmAfUmGfA/*/UNA-U/*/dT |
| 3481 | 432 | S | UNA-C/*/mGfCmCfGmUfGmCfAfGfCmCfCmAfAmUfGmAfG/*/UNA-U/*/dT |
| 3481 | 433 | S | UNA-G/*/mCfCmGfUmGfCmAfGfCfCmCfAmAfUmGfAmGfA/*/UNA-U/*/dT |
| 3481 | 434 | S | UNA-C/*/mCfGmUfGmCfAmGfCfCfCmAfAmUfGmAfGmAfG/*/UNA-U/*/dT |
| 3481 | 435 | S | UNA-C/*/mGfUmGfCmAfGmCfCfCfAmAfUmGfAmGfAmGfU/*/UNA-U/*/dT |
| 3481 | 436 | S | UNA-G/*/mUfGmCfAmGfCmCfCfAfAmUfGmAfGmAfGmUfG/*/UNA-U/*/dT |
| 3481 | 437 | S | UNA-U/*/mGfCmAfGmCfCmCfAfAfUmGfAmGfAmGfUmGfA/*/UNA-U/*/dT |
| 3481 | 438 | S | UNA-G/*/mCfAmGfCmCfCmAfAfUfGmAfGmAfGmUfGmAfC/*/UNA-U/*/dT |
| 3481 | 439 | S | UNA-C/*/mAfGmCfCmCfAmAfUfGfAmGfAmGfUmGfAmCfA/*/UNA-U/*/dT |
| 3481 | 440 | 5 | UNA-G/*/mCfCmCfAmAfUmGfAfGfAmGfUmGfAmCfAmAfC/*/UNA-U/*/dT |
| 3481 | 441 | S | UNA-C/*/mCfCmAfAmUfGmAfGfAfGmUfGmAfCmAfAmCfG/*/UNA-U/*/dT |
| 3481 | 442 | S | UNA-C/*/mCfAmAfUmGfAmGfAfGfUmGfAmCfAmAfCmGfA/*/UNA-U/*/dT |
| 3481 | 443 | S | UNA-C/*/mAfAmUfGmAfGmAfGfUfGmAfCmAfAmCfGmAfC/*/UNA-U/*/dT |
| 3481 | 444 | S | UNA-A/*/mAfUmGfAmGfAmGfUfGfAmCfAmAfCmGfAmCfU/*/UNA-U/*/dT |
| 3481 | 445 | S | UNA-A/*/mUfGmAfGmAfGmUfGfAfCmAfAmCfGmAfCmUfA/*/UNA-U/*/dT |
| 3481 | 446 | S | UNA-U/*/mGfAmGfAmGfUmGfAfCfAmAfCmGfAmCfUmAfC/*/UNA-U/*/dT |
| 3481 | 447 | S | UNA-G/*/mAfGmAfGmUfGmAfCfAfAmCfGmAfCmUfAmCfA/*/UNA-U/*/dT |
| 3481 | 448 | S | UNA-A/*/mGfAmGfUmGfAmCfAfAfCmGfAmCfUmAfCmAfU/*/UNA-U/*/dT |
| 3481 | 449 | S | UNA-G/*/mAfGmUfGmAfCmAfAfCfGmAfCmUfAmCfAmUfC/*/UNA-U/*/dT |
| 3481 | 450 | S | UNA-A/*/mGfUmGfAmCfAmAfCfGfAmCfUmAfCmAfUmCfA/*/UNA-U/*/dT |
| 3481 | 451 | S | UNA-G/*/mUfGmAfCmAfAmCfGfAfCmUfAmCfAmUfCmAfU/*/UNA-U/*/dT |
| 3481 | 452 | S | UNA-U/*/mGfAmCfAmAfCmGfAfCfUmAfCmAfUmCfAmUfC/*/UNA-U/*/dT |
| 3481 | 453 | S | UNA-G/*/mAfCmAfAmCfGmAfCfUfAmCfAmUfCmAfUmCfC/*/UNA-U/*/dT |
| 3481 | 454 | S | UNA-A/*/mCfAmAfCmGfAmCfUfAfCmAfUmCfAmUfCmCfC/*/UNA-U/*/dT |
| 3481 | 455 | S | UNA-C/*/mAfAmCfGmAfCmUfAfCfAmUfCmAfUmCfCmCfC/*/UNA-U/*/dT |

TABLE 14
UNA oligomers targeted to PDGFRB (Antisense (AS))

| REF | SEQ | | |
|---|---|---|---|
| POS | ID NO: | S/AS | PDGFRB (Antisense (AS)) (5′-3′) |
| 3481 | 456 | AS | mUfUmGfUmCfAmCfUmCfUmCmAmUfUmGfGmGfCmU/UNA-U/*/dT |
| 3481 | 457 | AS | mGfGmUfGmUfAmGfAmGfGmAmCmGfGmAfGmCfUmG/UNA-U/*/dT |
| 3481 | 458 | AS | mCfGmGfUmGfUmAfGmAfGmGmAmCfGmGfAmGfCmU/UNA-U/*/dT |
| 3481 | 459 | AS | mGfCmGfGmUfGmUfAmGfAmGmGmAfCmGfGmAfGmC/UNA-U/*/dT |
| 3481 | 460 | AS | mGfGmCfGmGfUmGfUmAfGmAmGmGfAmCfGmGfAmG/UNA-U/*/dT |
| 3481 | 461 | AS | mCfGmGfCmGfGmUfGmUfAmGmAmGfGmAfCmGfGmA/UNA-U/*/dT |
| 3481 | 462 | AS | mAfCmGfGmCfGmGfUmGfUmAmGmAfGmGfAmCfGmG/UNA-U/*/dT |
| 3481 | 463 | AS | mCfAmCfGmGfCmGfGmUfGmUmAmGfAmGfGmAfCmG/UNA-U/*/dT |
| 3481 | 464 | AS | mGfCmAfCmGfGmCfGmGfUmGmUmAfGmAfGmGfAmC/UNA-U/*/dT |
| 3481 | 465 | AS | mUfGmCfAmCfGmGfCmGfGmUmGmUfAmGfAmGfGmA/UNA-U/*/dT |
| 3481 | 466 | AS | mCfUmGfCmAfCmGfGmCfGmGmUmGfUmAfGmAfGmG/UNA-U/*/dT |
| 3481 | 467 | AS | mGfCmUfGmCfAmCfGmGfCmGmGmUfGmUfAmGfAmG/UNA-U/*/dT |
| 3481 | 468 | AS | mGfGmCfUmGfCmAfCmGfGmCmGmGfUmGfUmAfGmA/UNA-U/*/dT |
| 3481 | 469 | AS | mGfGmGfCmUfGmCfAmCfGmGmCmGfGmUfGmUfAmG/UNA-U/*/dT |
| 3481 | 470 | AS | mUfGmGfGmCfUmGfCmAfCmGmGmCfGmGfUmGfUmA/UNA-U/*/dT |
| 3481 | 471 | AS | mUfUmGfGmGfCmUfGmCfAmCmGmGfCmGfGmUfGmU/UNA-U/*/dT |
| 3481 | 472 | AS | mAfUmUfGmGfGmCfUmGfCmAmCmGfGmCfGmGfUmG/UNA-U/*/dT |
| 3481 | 476 | AS | mCfAmUfUmGfGmGfCmUfGmCmAmCfGmGfCmGfGmU/UNA-U/*/dT |
| 3481 | 474 | AS | mUfCmAfUmUfGmGfGmCfUmGmCmAfCmGfGmCfGmG/UNA-U/*/dT |
| 3481 | 475 | AS | mCfUmCfAmUfUmGfGmGfCmUmGmCfAmCfGmGfCmG/UNA-U/*/dT |
| 3481 | 476 | AS | mUfCmUfCmAfUmUfGmGfGmCmUmGfCmAfCmGfGmC/UNA-U/*/dT |
| 3481 | 477 | AS | mCfUmCfUmCfAmUfUmGfGmGmCmUfGmCfAmCfGmG/UNA-U/*/dT |
| 3481 | 478 | AS | mAfCmUfCmUfCmAfUmUfGmGmGmCfUmGfCmAfCmG/UNA-U/*/dT |
| 3481 | 479 | AS | mCfAmCfUmCfUmCfAmUfUmGmGmGfCmUfGmCfAmC/UNA-U/*/dT |
| 3481 | 480 | AS | mUfCmAfCmUfCmUfCmAfUmUmGmGfGmCfUmGfCmA/UNA-U/*/dT |
| 3481 | 481 | AS | mGfUmCfAmCfUmCfUmCfAmUmUmGfGmGfCmUfGmC/UNA-U/*/dT |
| 3481 | 482 | AS | mUfGmUfCmAfCmUfCmUfCmAmUmUfGmGfGmCfUmG/UNA-U/*/dT |
| 3481 | 483 | AS | mGfUmUfGmUfCmAfCmUfCmUmCmAfUmUfGmGfGmC/UNA-U/*/dT |
| 3481 | 484 | AS | mCfGmUfUmGfUmCfAmCfUmCmUmCfAmUfUmGfGmG/UNA-U/*/dT |
| 3481 | 485 | AS | mUfCmGfUmUfGmUfCmAfCmUmCmUfCmAfUmUfGmG/UNA-U/*/dT |
| 3481 | 486 | AS | mGfUmCfGmUfUmGfUmCfAmCmUmCfUmCfAmUfUmG/UNA-U/*/dT |
| 3481 | 487 | AS | mAfGmUfCmGfUmUfGmUfCmAmCmUfCmUfCmAfUmU/UNA-U/*/dT |
| 3481 | 488 | AS | mUfAmGfUmCfGmUfUmGfUmCmAmCfUmCfUmCfAmU/UNA-U/*/dT |
| 3481 | 489 | AS | mGfUmAfGmUfCmGfUmUfGmUmCmAfCmUfCmUfCmA/UNA-U/*/dT |
| 3481 | 490 | AS | mUfGmUfAmGfUmCfGmUfUmGmUmCfAmCfUmCfUmC/UNA-U/*/dT |
| 3481 | 491 | AS | mAfUmGfUmAfGmUfCmGfUmUmGmUfCmAfCmUfCmU/UNA-U/*/dT |
| 3481 | 492 | AS | mGfAmUfGmUfAmGfUmCfGmUmUmGfUmCfAmCfUmC/UNA-U/*/dT |
| 3481 | 493 | AS | mUfGmAfUmGfUmAfGmUfCmGmUmUfGmUfCmAfCmU/UNA-U/*/dT |
| 3481 | 494 | AS | mAfUmGfAmUfGmUfAmGfUmCmGmUfUmGfUmCfAmC/UNA-U/*/dT |
| 3481 | 495 | AS | mGfAmUfGmAfUmGfUmAfGmUmCmGfUmUfGmUfCmA/UNA-U/*/dT |
| 3481 | 496 | AS | mGfGmAfUmGfAmUfGmUfAmGmUmCfGmUfUmGfUmC/UNA-U/*/dT |
| 3481 | 497 | AS | mGfGmGfAmUfGmAfUmGfUmAmGmUfCmGfUmUfGmU/UNA-U/*/dT |
| 3481 | 498 | AS | mGfGmGfGmAfUmGfAmUfGmUmAmGfUmCfGmUfUmG/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 15. Table 15 shows “sense” sequences that are combined with an “antisense” sequence in Table 16. For example, SEQ ID NO:499 of Table 15 is combined with SEQ ID NO:517 of Table 16, SEQ ID NO:500 of Table 15 is combined with SEQ ID NO:518 of Table 16, etc.

TABLE 15
UNA oligomers targeted to PDGFRB (Sense (S))

| REF | SEQ | | |
|---|---|---|---|
| POS | ID NO: | S/AS | PDGFRB (Sense (S)) (5′-3′) |
| 3602 | 499 | S | UNA-A/*/mCfCmCfUmGfAmAfUfGfAmAfGmUfCmAfAmCfA/*/UNA-U/*/dT |
| 3602 | 500 | S | UNA-A/*/mGfCmCfAmGfCmUfCfCfAmCfCmCfUmGfAmAfU/*/UNA-U/*/dT |
| 3602 | 501 | S | UNA-G/*/mCfCmAfGmCfUmCfCfAfCmCfCmUfGmAfAmUfG/*/UNA-U/*/dT |
| 3602 | 502 | S | UNA-C/*/mCfAmGfCmUfCmCfAfCfCmCfUmGfAmAfUmGfA/*/UNA-U/*/dT |
| 3602 | 503 | S | UNA-C/*/mAfGmCfUmCfCmAfCfCfCmUfGmAfAmUfGmAfA/*/UNA-U/*/dT |
| 3602 | 504 | S | UNA-A/*/mGfCmUfCmCfAmCfCfCfUmGfAmAfUmGfAmAfG/*/UNA-U/*/dT |
| 3602 | 505 | S | UNA-G/*/mCfUmCfCmAfCmCfCfUfGmAfAmUfGmAfAmGfU/*/UNA-U/*/dT |
| 3602 | 506 | S | UNA-C/*/mUfCmCfAmCfCmCfUfGfAmAfUmGfAmAfGmUfC/*/UNA-U/*/dT |
| 3602 | 507 | S | UNA-U/*/mCfCmAfCmCfCmUfGfAfAmUfGmAfAmGfUmCfA/*/UNA-U/*/dT |
| 3602 | 508 | S | UNA-C/*/mCfAmCfCmCfUmGfAfAfUmGfAmAfGmUfCmAfA/*/UNA-U/*/dT |
| 3602 | 509 | S | UNA-C/*/mAfCmCfCmUfGmAfAfUfGmAfAmGfUmCfAmAfC/*/UNA-U/*/dT |
| 3602 | 510 | 5 | UNA-C/*/mCfCmUfGmAfAmUfGfAfAmGfUmCfAmAfCmAfC/*/UNA-U/*/dT |
| 3602 | 511 | S | UNA-C/*/mCfUmGfAmAfUmGfAfAfGmUfCmAfAmCfAmCfC/*/UNA-U/*/dT |
| 3602 | 512 | S | UNA-C/*/mUfGmAfAmUfGmAfAfGfUmCfAmAfCmAfCmCfU/*/UNA-U/*/dT |
| 3602 | 513 | S | UNA-U/*/mGfAmAfUmGfAmAfGfUfCmAfAmCfAmCfCmUfC/*/UNA-U/*/dT |
| 3602 | 514 | S | UNA-G/*/mAfAmUfGmAfAmGfUfCfAmAfCmAfCmCfUmCfC/*/UNA-U/*/dT |
| 3602 | 515 | S | UNA-A/*/mAfUmGfAmAfGmUfCfAfAmCfAmCfCmUfCmCfU/*/UNA-U/*/dT |
| 3602 | 516 | S | UNA-A/*/mUfGmAfAmGfUmCfAfAfCmAfCmCfUmCfCmUfC/*/UNA-U/*/dT |

TABLE 16
UNA oligomers targeted to PDGFRB (Antisense (AS))

| REF | SEQ | | |
|---|---|---|---|
| POS | ID NO: | S/AS | PDGFRB (Antisense (AS)) (5′-3′) |
| 3602 | 517 | AS | mUfGmUfUmGfAmCfUmUfCmAmUmUfCmAfGmGfGmU/UNA-U/*/dT |
| 3602 | 518 | AS | mAfUmUfCmAfGmGfGmUfGmGmAmGfCmUfGmGfCmU/UNA-U/*/dT |
| 3602 | 519 | AS | mCfAmUfUmCfAmGfGmGfUmGmGmAfGmCfUmGfGmC/UNA-U/*/dT |
| 3602 | 520 | AS | mUfCmAfUmUfCmAfGmGfGmUmGmGfAmGfCmUfGmG/UNA-U/*/dT |
| 3602 | 521 | AS | mUfUmCfAmUfUmCfAmGfGmGmUmGfGmAfGmCfUmG/UNA-U/*/dT |
| 3602 | 522 | AS | mCfUmUfCmAfUmUfCmAfGmGmGmUfGmGfAmGfCmU/UNA-U/*/dT |
| 3602 | 523 | AS | mAfCmUfUmCfAmUfUmCfAmGmGmGfUmGfGmAfGmC/UNA-U/*/dT |
| 3602 | 524 | AS | mGfAmCfUmUfCmAfUmUfCmAmGmGfGmUfGmGfAmG/UNA-U/*/dT |
| 3602 | 525 | AS | mUfGmAfCmUfUmCfAmUfUmCmAmGfGmGfUmGfGmA/UNA-U/*/dT |
| 3602 | 526 | AS | mUfUmGfAmCfUmUfCmAfUmUmCmAfGmGfGmUfGmG/UNA-U/*/dT |
| 3602 | 527 | AS | mGfUmUfGmAfCmUfUmCfAmUmUmCfAmGfGmGfUmG/UNA-U/*/dT |
| 3602 | 528 | AS | mGfUmGfUmUfGmAfCmUfUmCmAmUfUmCfAmGfGmG/UNA-U/*/dT |
| 3602 | 529 | AS | mGfGmUfGmUfUmGfAmCfUmUmCmAfUmUfCmAfGmG/UNA-U/*/dT |
| 3602 | 530 | AS | mAfGmGfUmGfUmUfGmAfCmUmUmCfAmUfUmCfAmG/UNA-U/*/dT |
| 3602 | 531 | AS | mGfAmGfGmUfGmUfUmGfAmCmUmUfCmAfUmUfCmA/UNA-U/*/dT |
| 3602 | 532 | AS | mGfGmAfGmGfUmGfUmUfGmAmCmUfUmCfAmUfUmC/UNA-U/*/dT |
| 3602 | 533 | AS | mAfGmGfAmGfGmUfGmUfUmGmAmCfUmUfCmAfUmU/UNA-U/*/dT |
| 3602 | 534 | AS | mGfAmGfGmAfGmGfUmGfUmUmGmAfCmUfUmCfAmU/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 17. Table 17 shows “sense” sequences that are combined with an “antisense” sequence in Table 18. For example, SEQ ID NO:535 of Table 17 is combined with SEQ ID NO:541 of Table 18, SEQ ID NO:536 of Table 17 is combined with SEQ ID NO:542 of Table 18, etc.

TABLE 17
UNA oligomers targeted to PDGFRB (Sense (S))

| REF | SEQ | | |
|---|---|---|---|
| POS | ID NO: | S/AS | PDGFRB (Sense (S)) (5′-3′) |
| 5564 | 535 | S | UNA-U/*/mCfAmCfCmUfAmGfGfUfUmUfAmCfAmAfAmUfA/*/UNA-U/*/dT |
| 5564 | 536 | S | UNA-A/*/mAfUmCfAmCfCmUfAfGfGmUfUmUfAmCfAmAfA/*/UNA-U/*/dT |
| 5564 | 537 | S | UNA-A/*/mUfCmAfCmCfUmAfGfGfUmUfUmAfCmAfAmAfU/*/UNA-U/*/dT |
| 5564 | 538 | S | UNA-C/*/mAfCmCfUmAfGmGfUfUfUmAfCmAfAmAfUmAfC/*/UNA-U/*/dT |
| 5564 | 539 | S | UNA-A/*/mCfCmUfAmGfGmUfUfUfAmCfAmAfAmUfAmCfU/*/UNA-U/*/dT |
| 5564 | 540 | S | UNA-C/*/mCfUmAfGmGfUmUfUfAfCmAfAmAfUmAfCmUfU/*/UNA-U/*/dT |

TABLE 18
UNA oligomers targeted to PDGFRB (Antisense (AS))

| REF | SEQ | | |
|---|---|---|---|
| POS | ID NO: | S/AS | PDGFRB (Antisense (AS)) (5′-3′) |
| 5564 | 541 | AS | mUfAmUfUmUfGmUfAmAfAmCmCmUfAmGfGmUfGmA/UNA-U/*/dT |
| 5564 | 542 | AS | mUfUmUfGmUfAmAfAmCfCmUmAmGfGmUfGmAfUmU/UNA-U/*/dT |
| 5564 | 543 | AS | mAfUmUfUmGfUmAfAmAfCmCmUmAfGmGfUmGfAmU/UNA-U/*/dT |
| 5564 | 544 | AS | mGfUmAfUmUfUmGfUmAfAmAmCmCfUmAfGmGfUmG/UNA-U/*/dT |
| 5564 | 545 | AS | mAfGmUfAmUfUmUfGmUfAmAmAmCfCmUfAmGfGmU/UNA-U/*/dT |
| 5564 | 546 | AS | mAfAmGfUmAfUmUfUmGfUmAmAmAfCmCfUmAfGmG/UNA-U/*/dT |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 19. Table 19 shows “sense” sequences that are combined with an “antisense” sequence in Table 20. For example, SEQ ID NO:547 of Table 19 is combined with SEQ ID NO:549 of Table 20, SEQ ID NO:548 of Table 19 is combined with SEQ ID NO:550 of Table 20.

TABLE 19
UNA oligomers targeted to PDGFRB (Sense (S))

| REF | SEQ | | |
|---|---|---|---|
| POS | ID NO: | S/AS | PDGFRB (Sense (S)) (5′-3′) |
| 3090 | 547 | S | UNA-G/*/mCfAmUfCmUfUmCfAfAfCm |
| | | | AfGmCfCmUfCmUfA/*/UNA-U/*/T |
| 3258 | 548 | S | uNA-C/*/mCfGmAfCmGfAmGfAfUfCm |
| | | | UfAmUfGmAfGmAfU/*/UNA-U/*/T |

TABLE 20
UNA oligomers targeted to
PDGFRB (Antisense (AS))

| REF | SEQ | | |
|---|---|---|---|
| POS | ID NO: | S/AS | PDGFRB (Antisense (AS)) (5′-3′) |
| 3090 | 549 | AS | mUfAmGfAmGfGmCfUmGfUmUm |
| | | | GmAfAmGfAmUfGmC/UNA-U/*/T |
| 3258 | 550 | AS | mAfUmCfUmCfAmUfAmGfAmUm |
| | | | CmUfCmGfUmCfGmG/UNA-U/*/T |

### UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Tables 21 and 22. The UNA oligomers shown in Tables 21 and 22 are targeted to PDGFRB sequences that are conserved between human and cynomolgus monkey.

Table 21 shows “sense” sequences that are combined with an “antisense” sequence in Table 22. For example, SEQ ID NO:551 of Table 21 is combined with SEQ ID NO:581 of Table 22, SEQ ID NO:552 of Table 21 is combined with SEQ ID NO:582 of Table 22, etc.

TABLE 21
UNA oligomers targeted to PDGFRB (Sense (S))

| REF | SEQ | | |
|---|---|---|---|
| POS | ID NO: | S/AS | PDGFRB (Sense (S)) (5′-3′) |
| 3142 | 551 | S | UNA-C/*/mCrUmGrCmUrCmUrGrGrGmArGmArUmCrUmUrC/*/UNA-U/*/dT |
| 622 | 552 | S | UNA-U/*/mCrUmGrAmCrCmUrGrCrUmCrGmGrGmUrUmCrA/*/UNA-U/*/dT |
| 2201 | 553 | S | UNA-G/*/mArGmUrAmCrAmUrCrUrAmCrGmUrGmGrAmCrC/*/UNA-U/*/dT |
| 2155 | 554 | S | UNA-C/*/mGrAmGrAmUrCmCrGrArUmGrGmArAmGrGmUrG/*/UNA-U/*/dT |
| 2895 | 555 | S | UNA-A/*/mGrGmUrGmGrCmCrArArUmGrGmCrAmUrGmGrA/*/UNA-U/*/dT |
| 3324 | 556 | S | UNA-C/*/mCrCmArGmCrUmGrGrUrGmCrUmGrCmUrUmCrU/*/UNA-U/*/dT |
| 1034 | 557 | S | UNA-A/*/mUrCmUrGmCrAmArArArCmCrAmCrCmArUmUrG/*/UNA-U/*/dT |
| 818 | 558 | S | UNA-U/*/mArCmArUmCrUmUrUrGrUmGrCmCrAmGrAmUrC/*/UNA-U/*/dT |
| 2789 | 559 | S | UNA-C/*/mCrUmUrAmCrGmArUrArAmCrUmArCmGrUmUrC/*/UNA-U/*/dT |
| 1194 | 560 | S | UNA-U/*/mGrGmUrCmArAmCrUrUrCmGrAmGrUmGrGmArC/*/UNA-U/*/dT |
| 2358 | 561 | S | UNA-A/*/mArGmUrGmGrCmCrGrUrCmArAmGrAmUrGmCrU/*/UNA-U/*/dT |
| 1306 | 562 | S | UNA-C/*/mArGmUrGmCrCmGrArGrUmUrAmGrAmArGmArC/*/UNA-U/*/dT |
| 3681 | 563 | S | UNA-A/*/mGrCmCrAmGrAmGrCrCrCmCrAmGrCmUrUmGrA/*/UNA-U/*/dT |
| 1478 | 564 | S | UNA-C/*/mArGmGrUmArGmUrGrUrUmCrGmArGmGrCmCrU/*/UNA-U/*/dT |
| 942 | 565 | S | UNA-U/*/mGrAmCrAmCrUmGrCrArCmGrAmGrAmArGmArA/*/UNA-U/*/dT |
| 4081 | 566 | S | UNA-C/*/mUrUmGrAmUrGmArCrCrCmArGmArAmUrCmUrA/*/UNA-U/*/dT |
| 4166 | 567 | S | UNA-G/*/mArGmGrUmGrGmUrArArAmUrUmArAmCrUmUrU/*/UNA-U/*/dT |
| 4206 | 568 | S | UNA-C/*/mUrCmArAmGrGmArArUrCmArUmArGmCrUmCrU/*/UNA-U/*/dT |
| 4309 | 569 | S | UNA-C/*/mArGmUrGmUrUmGrCrCrUmCrAmUrCmCrAmGrA/*/UNA-U/*/dT |
| 4420 | 570 | S | UNA-C/*/mUrGmArGmCrCmArArGrUmArCmArGmGrAmCrA/*/UNA-U/*/dT |
| 4558 | 571 | S | UNA-A/*/mArUmCrCmArUmCrCrArCmCrAmGrAmGrUmCrU/*/UNA-U/*/dT |
| 4594 | 572 | S | UNA-C/*/mCrGmCrAmUrCmUrGrUrGmArUmGrAmGrAmArU/*/UNA-U/*/dT |
| 4776 | 573 | S | UNA-C/*/mCrAmGrCmArCmUrArArCmArUmUrCmUrAmGrA/*/UNA-U/*/dT |
| 4801 | 574 | S | UNA-C/*/mArGmGrUmGrGmUrUrGrCmArCmArUmUrUmGrU/*/UNA-U/*/dT |
| 5185 | 575 | S | UNA-A/*/mCrAmGrUmUrAmUrGrUrCmUrUmGrUmArAmArA/*/UNA-U/*/dT |
| 5337 | 576 | 5 | UNA-C/*/mUrAmGrCmArGmUrGrArCmArUmCrUmCrAmUrU/*/UNA-U/*/dT |
| 5545 | 577 | S | UNA-A/*/mArUmArCmGrGmUrArCrCmArAmArGmArUmArU/*/UNA-U/*/dT |
| 5591 | 578 | S | UNA-G/*/mGrAmCrUmCrAmCrGrUrUmArAmCrUmCrAmCrA/*/UNA-U/*/dT |
| 5594 | 579 | S | UNA-C/*/mUrCmArCmGrUmUrArArCmUrCmArCmArUmUrU/*/UNA-U/*/dT |
| 5604 | 580 | S | UNA-C/*/mUrCmArCmArUmUrUrArUmArCmArGmCrAmGrA/*/UNA-U/*/dT |

TABLE 22
UNA oligomers targeted to PDGFRB (Antisense (AS))

| REF | SEQ | | |
|---|---|---|---|
| POS | ID NO: | S/AS | PDGFRB (Antisense (AS)) (5′-3′) |
| 3142 | 581 | AS | mGrAmArGmArUmCrUmCrCmCmAmGrAmGrCmArGmG/UNA-U/*/dT |
| 622 | 582 | AS | mUrGmArAmCrCmCrGmArGmCmAmGrGmUrCmArGmA/UNA-U/*/dT |
| 2201 | 583 | AS | mGrGmUrCmCrAmCrGmUrAmGmAmUrGmUrAmCrUmC/UNA-U/*/dT |
| 2155 | 584 | AS | mCrAmCrCmUrUmCrCmArUmCmGmGrAmUrCmUrCmG/UNA-U/*/dT |
| 2895 | 585 | AS | mUrCmCrAmUrGmCrCmArUmUmGmGrCmCrAmCrCmU/UNA-U/*/dT |
| 3324 | 586 | AS | mArGmArAmGrCmArGmCrAmCmCmArGmCrUmGrGmG/UNA-U/*/dT |
| 1034 | 587 | AS | mCrAmArUmGrGmUrGmGrUmUmUmUrGmCrAmGrAmU/UNA-U/*/dT |
| 818 | 588 | AS | mGrAmUrCmUrGmGrCmArCmAmAmArGmArUmGrUmA/UNA-U/*/dT |
| 2789 | 589 | AS | mGrAmArCmGrUmArGmUrUmAmUmCrGmUrAmArGmG/UNA-U/*/dT |
| 1194 | 590 | AS | mGrUmCrCmArCmUrCmGrAmAmGmUrUmGrAmCrCmA/UNA-U/*/dT |
| 2358 | 591 | AS | mArGmCrAmUrCmUrUmGrAmCmGmGrCmCrAmCrUmU/UNA-U/*/dT |
| 1306 | 592 | AS | mGrUmCrUmUrCmUrAmArCmUmCmGrGmCrAmCrUmG/UNA-U/*/dT |
| 3681 | 593 | AS | mUrCmArAmGrCmUrGmGrGmGmCmUrCmUrGmGrCmU/UNA-U/*/dT |
| 1478 | 594 | AS | mArGmGrCmCrUmCrGmArAmCmAmCrUmArCmCrUmG/UNA-U/*/dT |
| 942 | 595 | AS | mUrUmCrUmUrCmUrCmGrUmGmCmArGmUrGmUrCmA/UNA-U/*/dT |
| 4081 | 596 | AS | mUrAmGrAmUrUmCrUmGrGmGmUmCrAmUrCmArAmG/UNA-U/*/dT |
| 4166 | 597 | AS | mArAmArGmUrUmArAmUrUmUmAmCrCmArCmCrUmC/UNA-U/*/dT |
| 4206 | 598 | AS | mArGmArGmCrUmArUmGrAmUmUmCrCmUrUmGrAmG/UNA-U/*/dT |
| 4309 | 599 | AS | mUrCmUrGmGrAmUrGmArGmGmCmArAmCrAmCrUmG/UNA-U/*/dT |
| 4420 | 600 | AS | mUrGmUrCmCrUmGrUmArCmUmUmGrGmCrUmCrAmG/UNA-U/*/dT |
| 4558 | 601 | AS | mArGmArCmUrCmUrGmGrUmGmGmArUmGrGmArUmU/UNA-U/*/dT |
| 4594 | 602 | AS | mArUmUrCmUrCmArUmCrAmCmAmGrAmUrGmCrGmG/UNA-U/*/dT |
| 4776 | 603 | AS | mUrCmUrAmGrAmArUmGrUmUmAmGrUmGrCmUrGmG/UNA-U/*/dT |
| 4801 | 604 | AS | mArCmArAmArUmGrUmGrCmAmAmCrCmArCmCrUmG/UNA-U/*/dT |
| 5185 | 605 | AS | mUrUmUrUmArCmArAmGrAmCmAmUrAmArCmUrGmU/UNA-U/*/dT |
| 5337 | 606 | AS | mArAmUrGmArGmArUmGrUmCmAmCrUmGrCmUrAmG/UNA-U/*/dT |
| 5545 | 607 | AS | mArUmArUmCrUmUrUmGrGmUmAmCrCmGrUmArUmU/UNA-U/*/dT |
| 5591 | 608 | AS | mUrGmUrGmArGmUrUmArAmCmGmUrGmArGmUrCmC/UNA-U/*/dT |
| 5594 | 609 | AS | mArAmArUmGrUmGrAmGrUmUmAmArCmGrUmGrAmG/UNA-U/*/dT |
| 5604 | 610 | AS | mUrCmUrGmCrUmGrUmArUmAmAmArUmGrUmGrAmG/UNA-U/*/dT |

Any of the sequences in Tables 21 and 22 may contain one or more 2′-deoxy-2′-fluoro ribonucleotides.

### LAN-Containing UNA Oligomers Targeted to PDGFRB

Embodiments of this disclosure can provide oligomeric molecules that are active agents targeted to PDGFRB.

Examples of UNA oligomers of this disclosure that are targeted to PDGFRB are shown in Table 23. Table 23 shows sequentially “sense” and “antisense” pairs, for example, SEQ ID NO:335 and 341 are a “sense” and “antisense” pair.

TABLE 23
UNA oligomers targeted to PDGFRB (Sense (S)-Antisense (AS))

| REF | SEQ | | |
|---|---|---|---|
| POS | ID NO: | S/AS | PDGFRB (Sense (S)-Antisense (AS)) (5′-3′) |
| 5564 | 335 | S | UNA-U/*/mCrAmCrCmUrAmGrGrUrUmUrAmCrAmArAmUrA/*/UNA-U/*/T |
| 5564 | 341 | AS | mUrAmUrUmUrGmUrAmArAmCmCmUrAmGrGmUrGmA/UNA-U/*/T |
| 5564 | 613 | S | UNA-U/*/+ CrAmCrCmUrAmGrGrUrUmUrAmCrAmArAmUrA/*/UNA-U/*/T |
| 5594 | 614 | AS | mUrAmUrUmUrG + U + AmArAmCmCmUrAmGrGmUrGmA/UNA-U/*/T |
| 5594 | 579 | S | UNA-C/*/mUrCmArCmGrUmUrArArCmUrCmArCmArUmUrU/*/UNA-U/*/dT |
| 5594 | 609 | AS | mArAmArUmGrUmGrAmGrUmUmAmArCmGrUmGrAmG/UNA-U/*/T |

In Tables herein, rN refers to a ribonucleotide N, where N can be G, U, C, A, etc.; mN refers to a chemically-modified 2′ methoxy substituted (2′-OMe) ribonucleotide; an asterisk * between characters refers to a phosphorothioate linkage; dN refers to a deoxyribonucleotide; T and dT refer to a 2′-deoxy T nucleotide. Designations that may be used herein include mA, mG, mC, and mU, which refer to the 2′-O-Methyl modified ribonucleotides. +N refers to LNA (Locked nucleic acid), for example, +G would be a locked G.

The terms UNA-A, UNA-U, UNA-C, and UNA-G refer to UNA monomers. In some embodiments, a UNA monomer can be UNA-A (can be designated Ã), UNA-U (can be designated Ũ), UNA-C (can be designated (C̆) and UNA-G (can be designated Ğ).

### Methods for Treating NASH

This disclosure provides novel methods against nonalcoholic steatohepatitis. The therapeutic agents of this disclosure can be used as active pharmaceutical ingredients for ameliorating, preventing or treating nonalcoholic steatohepatitis. More particularly, therapeutic agents of this disclosure are active for gene silencing to suppress expression of PDGFRB. The methods of this disclosure can provide gene silencing agents that are active in vitro, and potent in vivo.

The active agents of this disclosure include UNA oligomeric molecules that can inhibit expression of PDGFRB. Oligomers of this disclosure can provide potent action against nonalcoholic steatohepatitis in a subject by downregulating and/or silencing expression of PDGFRB.

Methods of this disclosure include the treatment, amelioration and/or prevention of NASH disease, or one or more signs, symptoms or indications of NASH in a subject. A subject can be a human, or a mammal.

In the methods of this disclosure, a subject in need of treatment or prevention can be administered an effective amount of an oligomeric compound of this disclosure.

A subject in need may have any one or more of different signs and/or symptoms of NASH. Examples of signs and/or symptoms of NASH include fibrosis, steatosis, cell expansion or ballooning, and lobular and/or portal chronic inflammation.

A subject in need may have any one or more of the different signs and/or symptoms of NASH confirmed by a biopsy.

An effective amount of an oligomeric compound of this disclosure can be a dose ranging from 0.001 mg/kg to 50.0 mg/kg. The dose can be administered one or more times daily, or weekly.

In the methods of this disclosure, target mRNA expression can be reduced in a subject for at least 5 days. In certain embodiments, target mRNA expression can be reduced in a subject for at least 10 days, or 15 days, or 20 days, or 30 days, by administration of one or more doses of an effective amount of an oligomeric compound of this disclosure.

In the methods of this disclosure, the administration of an oligomeric compound may not result in an inflammatory response or may exhibit a reduced inflammatory response as compared to a conventional treatment, or a conventional siRNA.

In further embodiments, this disclosure includes methods for inhibiting expression of a target gene in a cell, by treating the cell with an oligomeric compound of this disclosure.

In additional embodiments, this disclosure includes methods for inhibiting expression of a target gene in a mammal, by administering to the mammal a composition containing an oligomeric compound of this disclosure.

An effective dose of an agent or pharmaceutical formulation of this disclosure, containing an oligomeric compound of this disclosure, can be an amount that, when introduced into a cell, is sufficient to cause suppression in the cell of the target of the oligomeric compound.

A therapeutically effective dose can be an amount of an agent or formulation that is sufficient to cause a therapeutic effect.

A therapeutically effective dose can be administered in one or more separate administrations, and by different routes.

As will be appreciated in the art, a therapeutically effective dose or a therapeutically effective amount can be determined based on the total amount of the therapeutic agent contained in the therapeutic composition.

A therapeutically effective amount can be sufficient to achieve a benefit to a subject in need, for example in treating, preventing and/or ameliorating a disease, or one or more signs, symptoms or indications of a disease or condition.

A therapeutically effective amount may be an amount sufficient to achieve a desired therapeutic and/or prophylactic effect.

In general, the amount of a therapeutic agent or composition administered to a subject in need thereof may depend upon the characteristics of the subject. Such characteristics include condition, disease severity, general health, age, sex, and body weight, among others.

One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of an oligomer. Pharmaceutical compositions comprising an oligomer can be administered at regular intervals, depending on the nature, severity and extent of the subject's condition.

In some embodiments, a therapeutically effective amount of an oligomer of the present disclosure may be administered periodically at regular intervals, for example, once every year, once every six months, once every four months, once every three months, once every two months, once a month, biweekly, weekly, daily, twice a day, three times a day, four times a day, five times a day, six times a day, or continuously.

In some embodiments, administering a therapeutically effective dose of a composition comprising an oligomer of this disclosure can result in decreased protein levels in a treated subject. In some embodiments, administering a composition comprising an oligomer of this disclosure can result in a 5%, 10%, 20%, 30%, 40%, 50%, 60%, 70%, 80%, 90%, 95%, or 100% decrease in protein levels relative to a baseline protein level in the subject prior to treatment.

In some embodiments, administering a therapeutically effective dose of a composition comprising an oligomer of this disclosure can result in reduced levels of one or more NASH disease markers.

A therapeutically effective in vivo dose of an oligomer of this disclosure can be about 0.001 mg/kg to about 500 mg/kg subject body weight.

In some embodiments, a therapeutically effective dose may be about 0.001-0.01 mg/kg body weight, or 0.01-0.1 mg/kg, or 0.1-1 mg/kg, or 1-10 mg/kg, or 10-100 mg/kg.

In some embodiments, an active oligomer of this disclosure can be provided at a dose ranging from about 0.1 to about 10 mg/kg body weight, or from about 0.5 to about 5 mg/kg, or from about 1 to about 4.5 mg/kg, or from about 2 to about 4 mg/kg.

A therapeutically effective in vivo dose of an active agent can be a dose of at least about 0.001 mg/kg body weight, or at least about 0.01 mg/kg, or at least about 0.1 mg/kg, or at least about 1 mg/kg, or at least about 2 mg/kg, or at least about 3 mg/kg, or at least about 4 mg/kg, or at least about 5 mg/kg, at least about 10 mg/kg, at least about 20 mg/kg, at least about 50 mg/kg, or more.

In some embodiments, an active agent can be provided at a dose of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, or about 6, 7, 8, 9, 10, 15, 20, 25, 50, 75, or 100 mg/kg.

siRNA Structures Targeted to PDGFRB

Embodiments of this disclosure further contemplate siRNA structures targeted to PDGFRB. As used herein, “siRNA” structures do not contain any UNA monomers. siRNA structures of this disclosure comprise RNA sequences, which may be chemically modified, that are targeted to suppress expression of PDGFRB. As used herein, the terms “agent” and “active agent” include siRNA structures, as well as UNA oligomers.

In further aspects, this disclosure provides siRNA structures targeted to PDGFRB.

A siRNA targeted to PDGFRB can be formed having a first strand and a second strand, each strand being 21 nucleotides in length. The first strand can have 19 contiguous nucleotides with a sequence of attached bases shown in Table 1 (sense), and two or more additional overhang nucleotides on the 3′ end. The second strand can have 19 contiguous nucleotides with a sequence of attached bases shown in Table 1 (same Ref Pos as first strand), and two or more additional overhang nucleotides on the 3′ end.

In some embodiments, siRNA overhang nucleotides can be any of NN, QQ, NQ, and QN. For example, NN can be dTdT.

For example, a siRNA of this disclosure based on Ref Pos 1094 is as follows, based on SEQ ID NOs: 3 and 53 of Table 1:

(sense)
SEQ ID NO: 611
CUCCAGGUGUCAUCCAUCAdTdT
(antisense)
SEQ ID NO: 612
UGAUGGAUGACACCUGGAGdTdT

### Pharmaceutical Compositions

In some aspects, the disclosure herein provides pharmaceutical compositions containing an oligomeric compound and a pharmaceutically acceptable carrier.

A pharmaceutical composition can be capable of local or systemic administration. In some aspects, a pharmaceutical composition can be capable of any modality of administration. In certain aspects, the administration can be intravenous, subcutaneous, pulmonary, intramuscular, intraperitoneal, dermal, oral, or nasal administration.

Embodiments of this disclosure include pharmaceutical compositions containing an oligomeric compound in a lipid formulation.

Additional embodiments of this disclosure include pharmaceutical compositions containing an oligomeric compound in a nanoparticle formulation.

In some embodiments, a pharmaceutical composition may comprise one or more lipids selected from cationic lipids, anionic lipids, sterols, pegylated lipids, and any combination of the foregoing.

In certain embodiments, a pharmaceutical composition can be substantially free of liposomes.

In further embodiments, a pharmaceutical composition can include nanoparticles.

Examples of nanoparticles include particles formed from lipid-like synthetic molecules.

In some embodiments, a nanoparticle may be formed with a composition containing a cationic lipid, or a pharmaceutically acceptable salt thereof, which may be presented in a lipid composition. A composition can comprise a nanoparticle, which may comprise one or more bilayers of lipid-like synthetic molecules.

A bilayer may further comprise a neutral lipid, or a polymer. A composition may comprise a liquid medium.

In some embodiments, a nanoparticle composition may encapsulate an agent, or oligomer of this disclosure.

In additional embodiments, a nanoparticle composition may comprise an oligomer of the present disclosure, along with a neutral lipid, or a polymer. A nanoparticle composition may entrap an oligomer of the present disclosure. In certain embodiments, a nanoparticle composition, as a delivery vehicle, can carry an oligomer of the present disclosure.

A nanoparticle composition may further comprise excipients for efficient delivery to cells or tissues, or for targeting cells or tissues, as well as for reducing immunological responses.

Some examples of lipid-like synthetic molecules, and nanoparticle compositions for delivery of an active molecule of this disclosure are given in WO/2015/074085 and U.S. patent application Ser. No. 15/387,067, each of which is hereby incorporated by reference in its entirety.

Examples of acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-napthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like. Acids which are generally considered suitable for the formation of pharmaceutically useful salts from basic pharmaceutical compounds are discussed, for example, by S. Berge et al, J. Pharmaceutical Sciences (1977) 66(1)1-19; P. Gould, International J. Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated by reference herein.

A pharmaceutical composition of this disclosure may include carriers, diluents or excipients as are known in the art. Examples of pharmaceutical compositions are described, for example, in Remington's Pharmaceutical Sciences, Mack Publishing Co. (A. R. Gennaro ed. 1985), and Remington, The Science and Practice of Pharmacy, 21st Edition (2005).

Examples of excipients for a pharmaceutical composition include antioxidants, suspending agents, dispersing agents, preservatives, buffering agents, tonicity agents, and surfactants, among others.

Examples of basic salts include ammonium salts, alkali metal salts such as sodium, lithium, and potassium salts, alkaline earth metal salts such as calcium and magnesium salts, salts with organic bases, for example, organic amines, such as benzathines, dicyclohexylamines, hydrabamines formed with N,N-bis(dehydroabietyl)ethylenediamine), N-methyl-D-glucamines, N-methyl-D-glucamides, t-butyl amines, and salts with amino acids such as arginine, lysine, and the like. Basic nitrogen-containing groups may be quarternized with agents such as lower alkyl halides, e.g., methyl, ethyl, propyl, and butyl chlorides, bromides, and iodides, dialkyl sulfates, e.g., dimethyl, diethyl, dibutyl, and diamyl sulfates), long chain halides, e.g., decyl, lauryl, myristyl, and stearyl chlorides, bromides, and iodides, arylalkyl halides, e.g., benzyl and phenethyl bromides, and others.

Compounds can exist in unsolvated and solvated forms, including hydrated forms. In general, the solvated forms, with pharmaceutically acceptable solvents such as water, ethanol, and the like, are equivalent to the unsolvated forms for the purposes of this disclosure. Compounds and salts, or solvates thereof, may exist in tautomeric forms, for example, as an amide or imino ether.

One or more lipid-like synthetic compounds may be combined with an oligomer of this disclosure to form microparticles, nanoparticles, liposomes, or micelles. A lipid-like synthetic compound can be a cationic lipid, or a cationic lipid-like molecule.

One or more lipid-like synthetic compounds and an oligomer of this disclosure may be combined with other lipid compounds, polymers, whether synthetic or natural, and other components, such as surfactants, cholesterol, carbohydrates, proteins, and/or lipids, to form particles. The particles may be further combined with one or more pharmaceutical excipients to form a pharmaceutical composition.

A lipid-like synthetic compound for forming nanoparticles may have a pKa in the range of approximately 5.5 to approximately 7.5, or between approximately 6.0 and approximately 7.0. In some embodiments, the pKa may be between approximately 3.0 and approximately 9.0, or between approximately 5.0 and approximately 8.0.

A composition containing one or more lipid-like synthetic compounds for forming nanoparticles may contain 30-70% of the lipid-like synthetic compounds, 0-60% cholesterol, 0-30% phospholipid, and 1-10% polyethylene glycol (PEG).

In some aspects, a composition containing one or more lipid-like synthetic compounds for forming nanoparticles may contain 30-40% of the lipid-like synthetic compounds, 40-50% cholesterol, and 10-20% PEG.

In certain embodiments, a composition containing one or more lipid-like synthetic compounds for forming nanoparticles may contain 50-75% of the lipid-like synthetic compounds, 20-40% cholesterol, 5 to 10% phospholipid, and 1-10% PEG.

In additional embodiments, a composition containing one or more lipid-like synthetic compounds for forming nanoparticles may contain 60-70% of the lipid-like synthetic compounds, 25-35% cholesterol, and 5-10% PEG.

A composition may contain up to 90% of a cationic lipid compound, and 2 to 15% helper lipid. Examples of a helper lipid include cholesterols, and neutral lipids such as DOPE.

A composition or formulation for delivery of an oligomer of this disclosure may be a lipid particle formulation.

A lipid particle formulation may contain 8-30% synthetic lipid, 5-30% helper lipid, and 0-20% cholesterol.

In some embodiments, a lipid particle formulation may contain 4-25% synthetic lipid, 4-25% helper lipid, 2 to 25% cholesterol, 10 to 35% cholesterol-PEG, and 5% cholesterol-amine.

In further embodiments, a lipid particle formulation may contain 2-30% synthetic lipid, 2-30% helper lipid, 1 to 15% cholesterol, 2 to 35% cholesterol-PEG, and 1-20% cholesterol-amine.

In additional embodiments, a lipid particle formulation may contain up to 90% synthetic lipid and 2-10% helper lipids.

In certain embodiments, a lipid particle formulation may contain 100% of one or more synthetic lipids.

Examples of cholesterol-based lipids include cholesterol, PEGylated cholesterol, DC-Chol (N,N-dimethyl-N-ethylcarboxamidocholesterol), and 1,4-bis(3-N-oleylamino-propyl)piperazine.

Examples of pegylated lipids include PEG-modified lipids. Examples of PEG-modified lipids include a poly(ethylene) glycol chain of up to 5 kDa in length covalently attached to a lipid with alkyl chain(s) of C₆-C₂₀ length.

Examples of a PEG-modified lipid include a derivatized ceramide, such as N-Octanoyl-Sphingosine-1-[Succinyl(Methoxy Polyethylene Glycol)-2000].

Examples of a PEG-modified or PEGylated lipid include PEGylated cholesterol or Dimyristoylglycerol (DMG)-PEG-2K.

### LUMINEX PBMc Cytokine Assay

A LUMINEX PBMc cytokine assay was used at a final UNA Oligomer concentration of 200 nM. R848 was 0.5 uM.

Human PBMC cells for same day transfection were plated at 2.5×10⁵ cells per well in a 96 well plate (10×10⁶ cells/vial). 10% FBS in RPMI, take 5 ml PRMI before adding FBS. 400 g at 12 mins centrifuge, resuspend cell in 10 mL RPMI+10% FBS. PBMC in 100 uL medium; 4 hrs before transfection.

Prepare mixture with DOTAP. For 10 reactions, 105 uL DOTAP+645 uL RPMI (no FBS). For 30 reactions, 315 uL DOTAP+1935 uL RPMI. For 30 reactions, 945 ul DOTAP+5805 ul RPMI for 30 min. 5 mins incubation time.

PROCARTAPLEX multiplex immunoassay was used following manufacturer's instructions. HU Basic Kit 96 test. HU IL-8/HU IL-10/HU TNFA/HU IFNG/HU MCP-1/Hu IP-10. Transfection conditional medium.

### UNA Oligomer In Vitro Transfection

Cell line: LX2 cell line for primary screening for hPDGFRb gene expression. 3T3 cell line for secondary screening for mPDGFRb gene expression.

Culture Medium: DEME+10% FBS+1×MEM NEAA. DMEM, HyClone Cat. #SH30243.01. FBS, HyClone Cat. #SH3007.03. MEM NEAA Thermo Cat #11140-050. TrypLE, Thermo Cat #12563-011.

Transfection medium: Opti-MEM I Reduced Serum Medium (Thermo Cat. #31985-070).

Transfection reagent: Lipofectamine RNAiMAX (Thermo Cat. #13778-100).

Transfection procedure: 1st day prepare cells. One day before the transfection, plate the cells in a 96-well plate at 3×10³ cells/well with 100 μl of DMEM+10% FBS+1×MEM NEAA and culture in a 37° C. incubator containing a humidified atmosphere of 5% CO2 in air. Next day, check the cell confluency before transfection (30%-50%) then replace the medium with 90 ul fresh complete DMEM medium. 2nd day prepare Oligomer dilution. Preparing Oligomer dilutions at 0, 5 nM, 50 nM, 500 nM concentrations from 10 uM stock solution in RNase free water. A: Prepare RNAiMAX+Opti-MEM. Mix 0.2 μl of Lipofectamine RNAiMAX with 4.8 μl of Opti-MEM I per each sample for 5 minutes at room temperature. B: Prepare diluted Oligomer+Opti-MEM in triplicate. Mix 1 μl of each diluted Oligomer with 4 μl of Opti-MEM I, wait for 5 minutes at room temperature. Prepare RNA-RNAiMAX complexes (A+B). Combine RNAiMAX solution with Oligomer solution half to half A+B. Mix gently without vortex. Incubate the mixture for 20 minutes at room temperature to allow the RNA-RNAiMAX complexes to form.

Transfection: Add the 10 μl of RNA-RNAiMAX complexes to a 96 well well by triplicate and shake. At this stage, the final concentration of the Oligomer would be 0, 50 pM, 500 pM, 5000 pM. Incubate the Oligomer transfected plate 24 hours at 37° C. incubator containing a humidified atmosphere of 5% CO₂ in air.

3^{rd} day TaqMan assay. Check cell density, best cell confluency should be ˜70%. Wash cell by use 1×PBS. Add cell-lysis buffer to lyste cell. Perform TaqMan KD assay.

Cell and Tissue-Based PDGFRB Silencing Analysis by qRT-PCR Assay

Cell and tissue-based PDGFRB silencing analysis was performed by qRT-PCR assay.

| Vendor | Product | Catalog | Assay ID |
|---|---|---|---|
| Thermo | hPDGRRb FAM/TG | 4351368 | Hs01019589_ ml |
| Thermo | Human RPLPO, | 4326314E | |
| | VIC ®/MGB probe | | |
| Thermo | mRRP19 VIC-MGB_ | 4448486 | Mm02601633_ gl |
| | PL/HKG | | |
| Thermo | mPDGFRb/FAM-MGB | 4351368 | Mm00435546_ ml |
| Thermo | Col1a1 | 4331182 | Mm00801666_g1 |
| Thermo | mF7 | 4331182 | Mm00487333_gl |
| Thermo | mRein | 4331182 | Mm00465200_m1 |
| Thermo | mClec4f | 4331182 | Mm00443934_ml |
| Thermo | mTek | 4331182 | Mm00443243_m1 |
| Thermo | TaqMan RNA-To-Ct | P/N | |
| | 1-step Master Mix | 4392656 | |
| LifeOMe | EZCt Cells-2-Ct Direct | EZCt-100 | |
| | | m1 | |

In vitro. Cell lines LX2, 3T3, Rat Primary cell. Medium: DMEM with 10% FBS and 1% Pen/Strep/25 nM HEPES (P4/P0 to P5/P1).

In vivo with RNA isolation. RA1 containing 15 mM DTT. Dissolve 500 mg DTT powder into 216 ml RA1. rDNase reaction. Tissue homogenizing. Bind the RNA onto membrane. Desalt membrane. DNase incubation. Wash membrane. Dry RNA plate. Elute RNA. Determine RNA unit quantity. RT-qPCR assay and data analysis.

### Luciferase Reporter Assay

Luciferase-based reporter plasmid was constructed based on psiCHECK™2 vector (Promega, Madison, Wis.). Reporter p(1-20) was generated with oligonucleotides containing the sequence from position 1 through 2500 relative to Eco RI digestion site cloned into the multiple cloning region downstream of the stop codon of the SV40 promoted Renilla luciferase gene in psiCHECK™2, which made the expression of Renilla luciferase gene under the regulation of the artificial 3′UTR sequence. Renilla luciferase activity was then used as an indicator of the effect of the artificial 3′UTR on transcript stability and translation efficiency. The psiCHECK™-2 Vector also contained a constitutively expressed Firefly luciferase gene, which served as an internal control to normalize transfection efficiency.

A total of 5,000 HepB3 cells (American Type Culture Collection) were plated onto a well of 96-well plate one day before the transfection. The cells were incubated at 37° C. in 100 μl of DMEM (Life Technologies, Carlsbad, Calif.) supplemented with 0.1 mM nonessential amino acids and 10% FBS (Life Technologies, Carlsbad, Calif.). The culture medium was changed to 90 μl of fresh medium just before the transfection. The reporter plasmid and UNA Oligomer were co-transfected with transfection reagent, Lipofectamine™ 3000 (Life Technologies, Carlsbad, Calif.) was used to transfect reporter plasmid (100 ng) and a various amount of UNA Oligomer together with P3000 into the cells according to manufacturer's instruction.

Dual-Luciferase Reporter Assay System (DLR assay system, Promega, Madison, Wis.) was used to perform dual-reporter assays on psiCHECK2 based reporter systems. Twenty-four hours after transfection, the cells were washed gently with phosphate buffered saline once. A 50 μl well of Passive Lysis Buffer (Promega, Madison, Wis.) was added to the cells and incubated with gentle rocking for 20 min at room temperature. Luciferase activities were measured using Cytation 3 imaging reader (BioTek, Winooski, Vt.) and the effect of the UNA Oligomer on reporter expression was calculated based on ratio of Renilla/Firefly to normalize cell number and transfection efficiency.

### Examples

Example 1: Activity of UNA Oligomers for suppressing PDGFRB. The PDGFRB inhibitory effect of UNA oligomers was observed in human hepatic stellate cells (LX-2). The IC50 for inhibition of target expression for several of the UNA oligomeric compounds is shown in Table 24.

TABLE 24
PDGFRB inhibitory effect of UNA oligomers

| | Cell LX2 |
|---|---|
| UNA Oligomer # (Ref Pos) | IC50 (pM) |
| #8 (3258) SEQ ID NO: 115/116 | 5-50 |
| #38 (3481) SEQ ID NO: 243/286 | 5-50 |
| #38 (3481) SEQ ID NO: 249/292 | 5-50 |
| #38 (3481) SEQ ID NO: 251/294 | 5-50 |
| #38 (3481) SEQ ID NO: 252/295 | 5-50 |
| #40 (3602) SEQ ID NO: 305/323 | 5-50 |
| #40 (3602) SEQ ID NO: 309/327 | 5-50 |
| #40 (3602) SEQ ID NO: 509/527 | 5-50 |
| #40 (3602) SEQ ID NO: 312/330 | 5-50 |
| #40 (3602) SEQ ID NO: 314/332 | 5-50 |
| #40 (3602) SEQ ID NO: 514/532 | 5-50 |
| #48 (5564) SEQ ID NO: 335/341 | <5 |
| #48 (5564) SEQ ID NO: 336/342 | 5-50 |
| #48 (5564) SEQ ID NO: 337/343 | 5-50 |
| #48 (5564) SEQ ID NO: 537/543 | 5-50 |
| #48 (5564) SEQ ID NO: 338/344 | 5-50 |
| #48 (5564) SEQ ID NO: 538/544 | <5 |
| #48 (5564) SEQ ID NO: 339/345 | 5-50 |
| #48 (5564) SEQ ID NO: 539/545 | 5-50 |
| #48 (5564) SEQ ID NO: 340/346 | 5-50 |
| #48 (5564) SEQ ID NO: 540/546 | 5-50 |

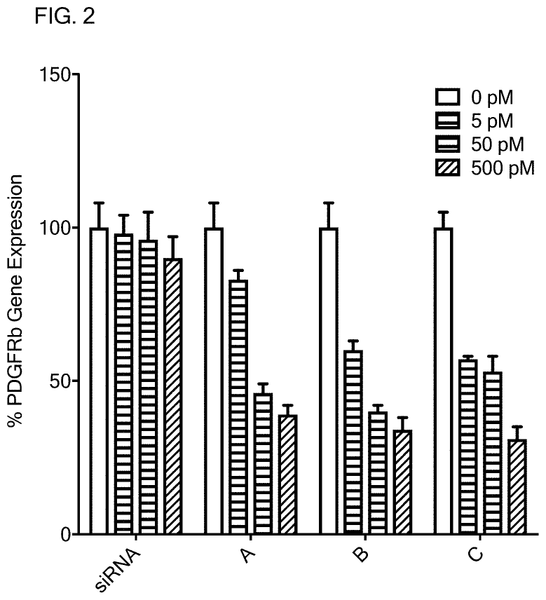

Example 2: Activity of UNA Oligomers for suppressing PDGFRB. The PDGFRB inhibitory effect of UNA oligomers was observed in rat primary hepatic stellate cells (RHSteC). FIG. 2 shows relative PDGFRB gene expression knockdown in rat primary hepatic stellate cells (RHSteC, ScienCell Research Laboratories, cat #R5300-a, lot #20034) for selected UNA Oligomers based on structure #48 (Ref Pos 5564). Oligomer structures 1 (SEQ ID NO:103/104), 3 (SEQ ID NO:107/108), and 5 (SEQ ID NO:111/112) showed surprisingly superior PDGFRB knockdown as compared to a conventional siRNA based on the same reference position.

Example 3: Selectivity of UNA Oligomers for suppressing PDGFRB over PDGFRA. The inhibitory effect of UNA oligomeric compounds was surprisingly selective for suppressing PDGFRB over PDGFRA.

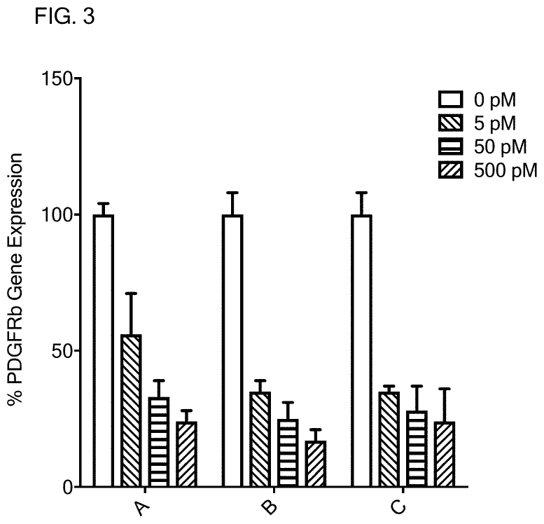

FIG. 3 shows relative PDGFRB gene expression knockdown in human hepatic stellate cells (LX-2) for selected UNA Oligomers based on structure #48 (Ref Pos 5564). Oligomer structures A (SEQ ID NO:111/112), B (SEQ ID NO:103/104), and C (SEQ ID NO:107/108) showed superior PDGFRB knockdown.

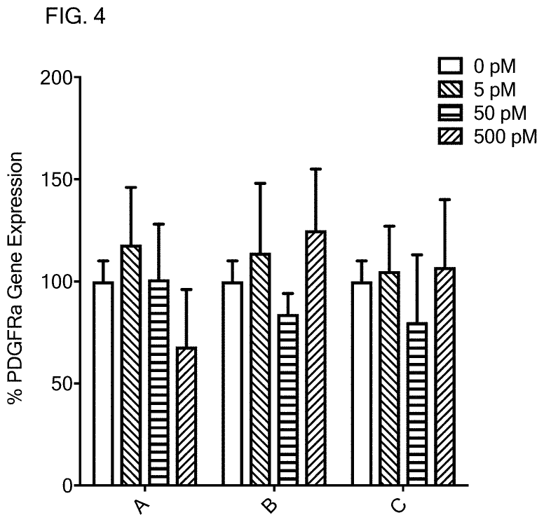

FIG. 4 shows relative PDGFRA gene expression knockdown in human hepatic stellate cells (LX-2) for selected UNA Oligomers based on structure #48 (Ref Pos 5564). As compared to FIG. 3, Oligomer structures A (SEQ ID NO:111/112), B (SEQ ID NO:103/104), and C (SEQ ID NO:107/108) did not substantially knockdown PDGFRA gene expression. Thus, the UNA Oligomers were surprisingly selective for reducing gene expression of PDGFRB over that of PDGFRA.

Example 4: Reduced immune response of UNA Oligomers in suppressing PDGFRB. UNA oligomeric compounds exhibited surprisingly reduced IL-8 response in suppressing expression of PDGFRB.

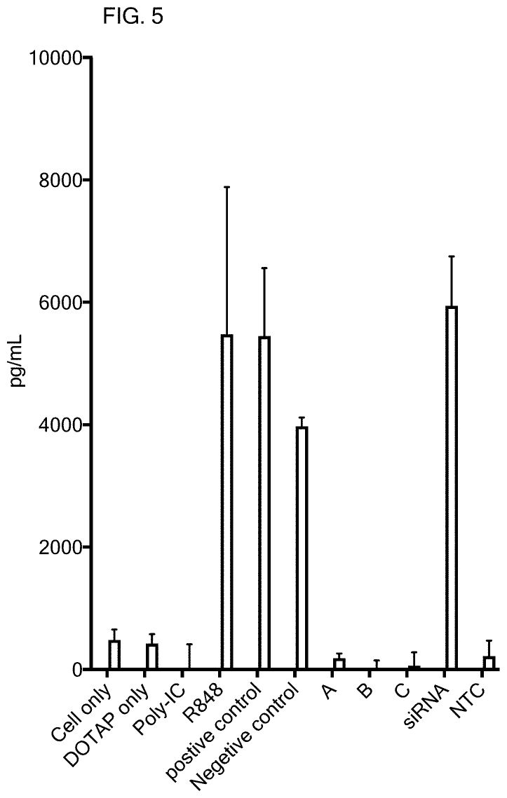

FIG. 5 shows an IL-8 assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced IL-8 stimulation as compared to a conventional siRNA based on the same reference position.

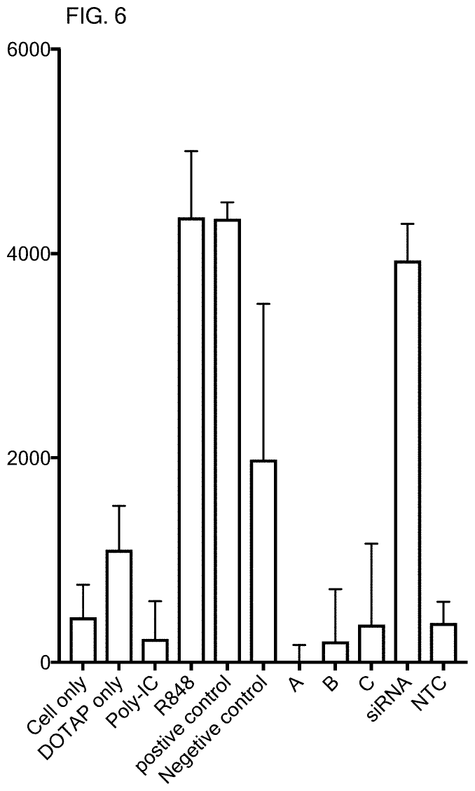

FIG. 6 shows an IL-8 assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced IL-8 stimulation as compared to a conventional siRNA based on the same reference position.

Example 5: Reduced immune response of UNA Oligomers in suppressing PDGFRB. UNA oligomeric compounds exhibited surprisingly reduced TNFa response in suppressing expression of PDGFRB.

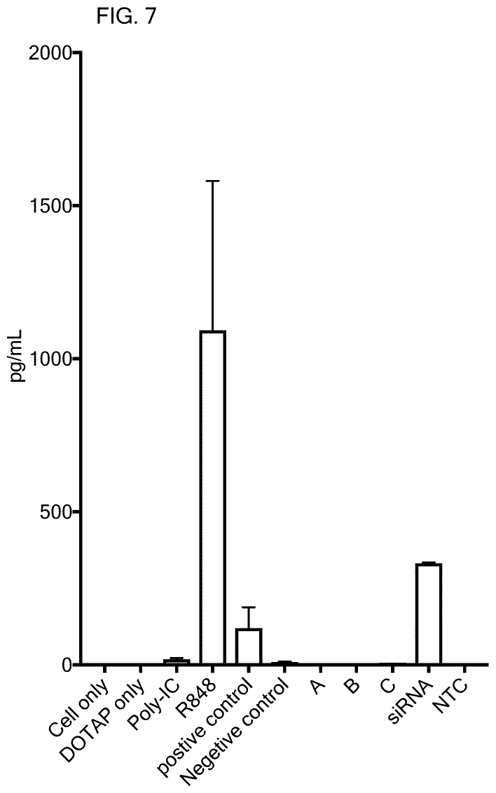

FIG. 7 shows a TNFa assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced TNFa stimulation as compared to a conventional siRNA based on the same reference position.

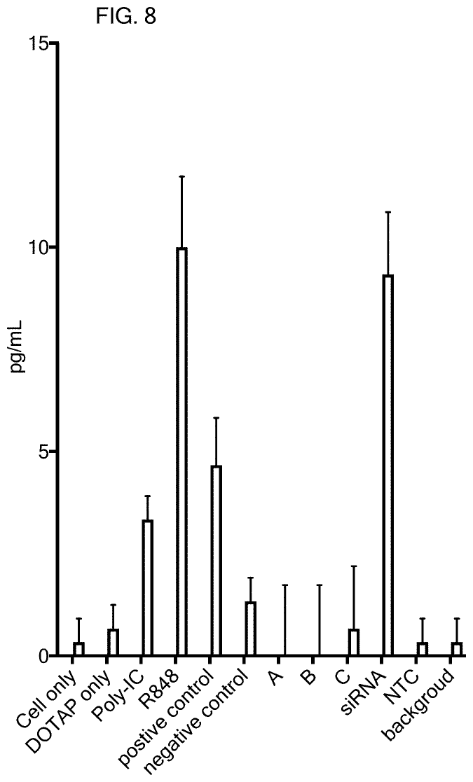

FIG. 8 shows an TNFa assay in hPBMC for Oligomer structures A (SEQ ID NO:111/112), B (SEQ ID NO:103/104), and C (SEQ ID NO:107/108) at 200 nM (n=3). Oligomer structures A, B and C showed surprisingly reduced TNFa stimulation as compared to a conventional siRNA based on the same reference position.

Example 6: Potency of UNA Oligomers for suppressing PDGFRB in vivo. The PDGFRB inhibitory effect of UNA oligomers administered using a lipid nanoparticle formulation was observed in vivo mouse.

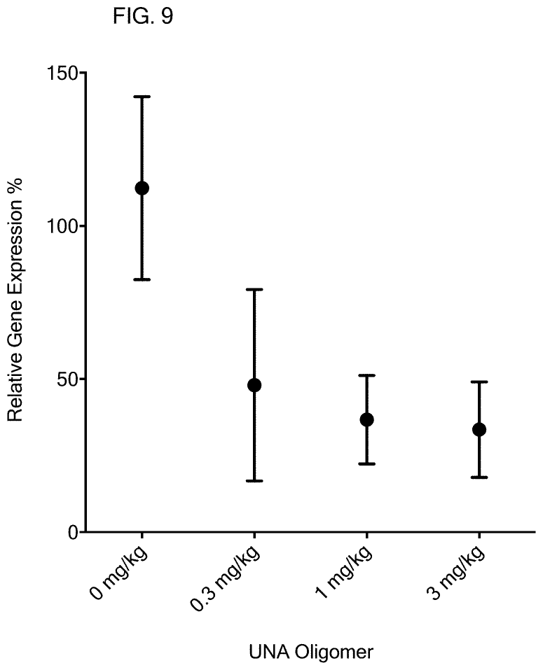

FIG. 9 shows relative PDGFRB gene expression knockdown in MDR2 knockout mice in vivo for a UNA Oligomer based on structure #48 (Ref Pos 5564). Oligomer B (SEQ ID NO:103/104) was formulated in a lipid nanoparticle formulation based on ATX126 and administered up to 3 mg/kg. MDR2 knockout mice, FVB.129P2-Abcb4^{tm1Bor}/J, Stock #002539, Jackson Laboratory.

Protocol for lipid nanoparticle formulation. Lipid-based nanoparticles were prepared by mixing appropriate volumes of an aqueous phase containing Oligomer duplexes with lipids in ethanol, using a Nanoassemblr microfluidic device, followed by downstream processing. For the formulation preparation, the desired amount of Oligomer was dissolved in 2 mM citric acid buffer with 9% sucrose, pH 3.5. Lipids at the desired molar ratio were dissolved in ethanol. The molar percentage ratio for the constituent lipids was 58% ATX (proprietary ionizable amino lipids), 7% DSPC (1,2-distearoyl-sn-glycero-3-phosphocholine) (Avanti Polar Lipids), 33.5% cholesterol (Avanti Polar Lipids), and 1.5% DMG-PEG (1,2-Dimyristoylsn-glycerol, methoxypolyethylene glycol, PEG chain molecular weight: 2000) (NOF America Corporation). At a flow ratio of 1:3 ethanol: aqueous phases, the solutions were combined in the microfluidic device (Precision NanoSystems). The total combined flow rate was 12 mL/min. The mixed material was then diluted three times with 10 mM Tris, 50 mM NaCl and 9% sucrose buffer. The diluted LNP slurry was concentrated by tangential flow filtration with hollow fiber membranes (mPES Kros membranes, Spectrum Laboratories), and then diafiltration with 10 mM Tris, 50 mM NaCl and 9% sucrose buffer. Particle size was determined by dynamic light scattering (ZEN3600, Malvern Instruments). Encapsulation efficiency was calculated by determining unencapsulated Oligomer content by measuring the fluorescence upon the addition of RiboGreen (Molecular Probes) to the LNP slurry (Fi) and comparing this value to the total RNA content that was obtained upon lysis of the LNPs by 1% Triton X-100 (Ft), where % encapsulation=(Ft−Fi)/Ft×100.

Protocol for test article administration. Test/Control Articles were administered by a single bolus intravenous injection on Day 0 at time 0. The final dose volume was calculated based on the individual body weights from the most recent measurement. A 1 ml dosing syringe (BD #329654) was loaded with the appropriate volume of test article and capped with a 27-gauge needle (BD #305136). Mice were placed in a physical restraint with full access to the tail. The test article was administered intravenously through the lateral tail vein.

Blood was collected by cardiac puncture and processed to serum. Livers were harvested and separated into two aliquots (˜30 mg, remaining) and flash frozen in liquid nitrogen.

Blood samples were allowed to clot for at least 30 minutes before spun down and processed to serum.

Example 7: Activity of UNA Oligomers for suppressing PDGFRB in different species. Examples of UNA oligomers of this disclosure that were targeted to PDGFRB sequences that are conserved between human and cynomolgus monkey were active for suppressing expression of PDGFRB.

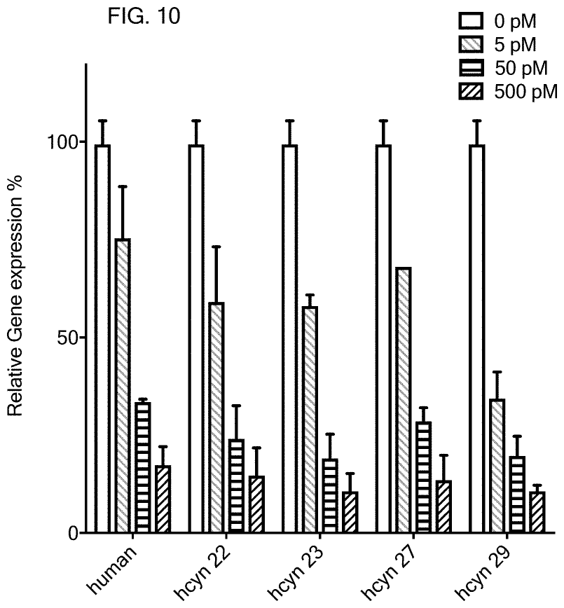

FIG. 10 shows relative PDGFRB gene expression knockdown in human hepatic stellate cells (LX-2) for selected UNA Oligomers. Oligomer structures hcyn22 (Ref Pos 4594) (SEQ ID NO:572/602), hcyn23 (Ref Pos 4776) (SEQ ID NO:573/603), hcyn27 (Ref Pos 5545) (SEQ ID NO:577/607), and hcyn29 (Ref Pos 5594) (SEQ ID NO:579/609) showed superior PDGFRB knockdown as compared to Oligomer B (SEQ ID NO:103/104). Thus, the hcyn Oligomers are cross reactive in human and cynomolgus monkey.

Example 8: Activity of siRNAs for suppressing PDGFRB. Certain siRNA sequences, which contained only natural nucleotides, showed useful PDGFRB knockdown activity. The siRNAs are not UNA Oligomers.

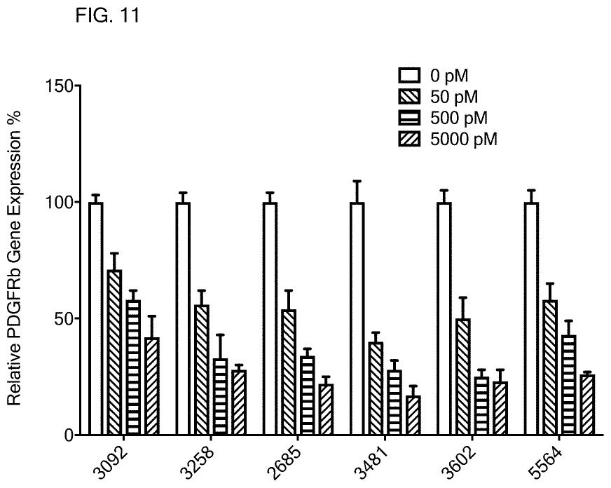

FIG. 11 shows relative PDGFRB gene expression knockdown in human hepatic stellate cells (LX-2) 24 hr post transfection for selected siRNAs based on sequences #6 (Ref Pos 3092) (SEQ ID NO:8/58), #8 (Ref Pos 3258) (SEQ ID NO:10/60), #23 (Ref Pos 2685) (SEQ ID NO:25/75), #38 (Ref Pos 3481) (SEQ ID NO:40/90), #40 (Ref Pos 3602) (SEQ ID NO:42/92), and #48 (Ref Pos 5564) (SEQ ID NO:50/100), each of which had two dTdT 3′ overhangs. These siRNAs contained only natural nucleotides and showed useful PDGFRB knockdown.

Thus, certain siRNA sequences, which contained only natural nucleotides, showed useful PDGFRB knockdown activity.

Example 9: Effect of LNA-containing UNA Oligomer on PDGFRB Expression in LX2 Cell. The PDGFRB inhibitory effect of UNA oligomers observed in human hepatic stellate cells (LX-2) for LNA-containing UNA oligomers is shown in Table 25. The IC50 comparison of PRb48-1-CM1 for inhibition of target expression for the LNA-containing UNA oligomeric compounds is shown in Table 26.

TABLE 25
LNA-containing UNA Oligomers

| siRNA ID | UNA Oligomer # (Ref Pos) |
|---|---|
| PRb48-1-CM1 | (5564) SEQ ID NO: 335/341 |
| LNAsi-7 | (3481) SEQ ID NO: 335/614 |
| LNAsi-9 | (3481) SEQ ID NO: 613/614 |
| hcyn-29-CM1 | (5594) SEQ ID NO: 579/609 |

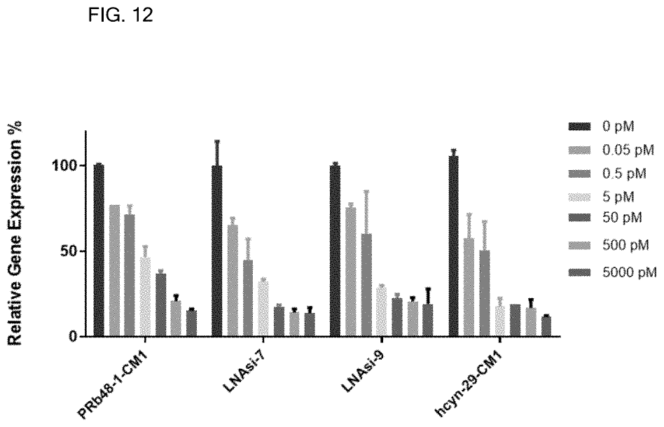

FIG. 12 shows relative PDGFRB gene expression knockdown in human hepatic stellate cells (LX-2) for selected LNA-containing UNA Oligomers. Oligomer LNA-containing UNA oligomer structures LNAsi-7 (Ref Pos 5564) (SEQ ID NO:335/614) and LNAsi-9 (Ref Pos 5564) (SEQ ID NO:613/614), and hcyn-29-CM1 (Ref Pos 5594) (SEQ ID NO:579/609) showed a substantial change of PDGFRB expression knockdown as compared to PRb48-1-CM1 (Ref Pos 5564) (SEQ ID NO:335/341).

TABLE 26
Effect of LNA-containing UNA Oligomers on PDGFRB
Expression in LX2 Cells

| | | Fold Change as |
|---|---|---|
| | Cell LX2 | compared to |
| siRNA ID | IC50 (pM) | PRb48-1-CM1 |
| PRb48-1-CM1 | 6.44 | N/A |
| LNAsi-7 | 0.34 | 18.94 |
| LNAsi-9 | 1.04 | 6.19 |
| hcyn-29-CM1 | 0.44 | 14.64 |

Example 9: Effect of LNA-containing UNA Oligomer on Cytotoxicity in LX2 Cells. The cytotoxicity effect of UNA oligomers observed in human hepatic stellate cells (LX-2) for several LNA-containing UNA oligomers is shown in Table 25.

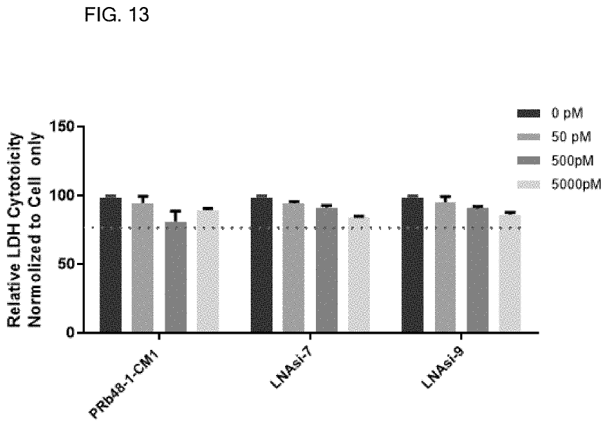

FIG. 13 shows relative LDH cytotoxicity in human hepatic stellate cells (LX-2) for selected LNA-containing UNA Oligomers. Oligomer LNA-containing structures LNAsi-7 (Ref Pos 5564) (SEQ ID NO:335/614) and LNAsi-9 (Ref Pos 5564) (SEQ ID NO:613/614) showed superior cytotoxicity as compared to PRb48-1-CM1 (Ref Pos 5564) (SEQ ID NO:335/341).

Example 10: Effect of LNA-Containing UNA Oligomer on Cell Viability of LX2 Cells. The cytotoxcity effect of UNA oligomers observed in human hepatic stellate cells (LX-2) for several LNA-containing UNA oligomers is shown in Table 25.

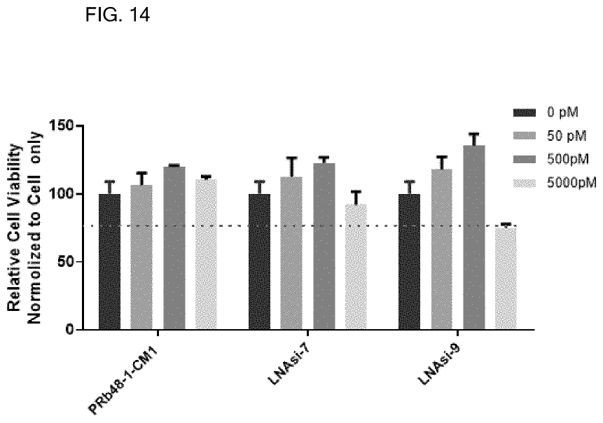

FIG. 14 shows relative cell viability in human hepatic stellate cells (LX-2) for selected LNA-containing UNA Oligomers. Oligomer LNA-containing structures LNAsi-7 (Ref Pos 5564) (SEQ ID NO:335/614) and LNAsi-9 (Ref Pos 5564) (SEQ ID NO:613/614) showed superior cell viability as compared to PRb48-1-CM1 (Ref Pos 5564) (SEQ ID NO:335/341).

All publications, patents and literature specifically mentioned herein are incorporated by reference for all purposes.

It is understood that this disclosure is not limited to the particular methodology, protocols, materials, and reagents described, as these may vary. It is also to be understood that the terminology used herein is for the purpose of describing particular embodiments only and is not intended to limit the scope of the present disclosure, which will be encompassed by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprises,” “comprising”, “containing,” “including”, and “having” can be used interchangeably.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever.

All of the features disclosed in this specification may be combined in any combination. Each feature disclosed in this specification may be replaced by an alternative feature serving the same, equivalent, or similar purpose.

1. A compound comprising a first strand and a second strand, each of the strands being 19-29 monomers in length, the monomers comprising UNA monomers and nucleic acid monomers, wherein the first strand is a passenger strand for RNA interference and the second strand is a guide strand for RNA interference, and wherein the compound comprises at least one of the following sense-antisense pairs targeted to PDGFRB:
(#48) SEQ ID NO: 335 and 341;
(#48) SEQ ID NO: 336 and 342;
(#48) SEQ ID NO: 337 and 343;
(#48) SEQ ID NO: 338 and 344;
(#48) SEQ ID NO: 339 and 345;
(#48) SEQ ID NO: 340 and 346;
(LNAsi-7) SEQ ID NO: 335 and 614;
(LNAsi-9) SEQ ID NO: 613 and 614; and
(hcyn-29-CM1) SEQ ID NO: 579 and 609.

2. The compound of claim 1, wherein any one or more of the nucleic acid monomers is chemically-modified.

3. The compound of claim 1, wherein the compound is conjugated to a delivery moiety.

4. The compound of claim 1, wherein the compound is conjugated to a delivery moiety that binds to a glycoprotein receptor.

5. The compound of claim 1, wherein the compound is conjugated to a delivery moiety that binds to a glycoprotein receptor, wherein the delivery moiety comprises a galactose, a galactosamine, or a N-acetylgalactosamine.

6. The compound of claim 1, wherein the compound is conjugated to a GalNAc delivery moiety.

7. The compound of claim 1, wherein the compound is conjugated to a cholesterol or LNA delivery moiety.

8. The compound of claim 1, wherein the compound is conjugated to a delivery moiety at an end of the compound and has increased uptake in the liver as compared to an unconjugated compound.

9. The compound of claim 1, further comprising a lipid nanoparticle composition encapsulating the compound.

10. A pharmaceutical composition comprising one or more compounds of claim 1 and a pharmaceutically acceptable carrier.

11. The pharmaceutical composition of claim 10, comprising:
(i) a lipid formulation;
(ii) one or more lipids selected from cationic lipids, anionic lipids, sterols, pegylated lipids, and any combination thereof; or
(iii) both (i) and (ii).

12. The pharmaceutical composition of claim 10, wherein the pharmaceutically acceptable carrier comprises lipid nanoparticles or liposomes.

13. A method for treating non-alcoholic steatohepatitis (NASH) in a subject, the method comprising administering to the subject an effective amount of the pharmaceutical composition of claim 10.

14. The method of claim 13, comprising inhibiting expression of PDGFRB in the subject.

15. The method of claim 13, further comprising preventing, ameliorating or treating a disease or condition associated with NASH in the subject.

16. The method of claim 13, wherein administration of the pharmaceutical composition reduces liver size or liver steatosis in the subject.

17. The method of claim 13, wherein the reduction in liver size or liver steatosis is measured by a biopsy or by a non-invasive method.
